(19) United States
(12) Patent Application Publication
(10) Pub. No.: US 2023/0000864 A1
(43) Pub. Date: Jan. 5, 2023

(54) SOLID DOSE PHARMACEUTICAL COMPOSITION

(71) Applicant: SUMITOMO PHARMA ONCOLOGY, INC., Cambridge, MA (US)

(72) Inventors: Steven L. WARNER, Sandy, UT (US); Jason Marc FOULKS, Sandy, UT (US); Paul FLYNN, Citrus Heights, CA (US)

(73) Assignee: SUMITOMO PHARMA ONCOLOGY, INC., Cambridge, MA (US)

(21) Appl. No.: 17/778,284

(22) PCT Filed: Nov. 20, 2020
(86) PCT No.: PCT/US2020/061629

§ 371 Date: May 19, 2022

Related U.S. Application Data
(60) Provisional application No. 62/939,489, filed on Nov. 22, 2019.

(51) Int. Cl. A61K 31/506; A61K 47/38; A61K 47/42; A61K 9/48

(52) U.S. Cl. CPC A61K 31/506; A61K 47/38; A61K 47/42; A61K 9/4816

(57) ABSTRACT

The present disclosure provides pharmaceutical compositions comprising a compound having activity as an ACVR1 (ALK2) or ALK5 inhibitor.

### CROSS REFERENCE TO RELATED APPLICATIONS

This application claims the benefit of U.S. Provisional Application No. 62/939,489, filed Nov. 22, 2019, which is hereby incorporated by reference in its entirety.

### FIELD OF THE DISCLOSURE

The present disclosure provides pharmaceutical compositions comprising a compound having activity as an ACVR1 inhibitor.

### BACKGROUND OF THE DISCLOSURE

Compounds having activity as inhibitors of one or more of activin receptor-like kinases (ALKs) and Janus kinases (JAKs) are disclosed in WO 2014/151871, incorporated herein by reference. The compounds demonstrate activity as inhibitors of, at least, ALK2 and JAK2. Kinase subtypes may be known under alternative names. As an example, ALK2 is also known as activin A receptor, type 1 (ACVR1).

As an inhibitor of ALK2 (ACVR1), the compounds of WO 2014/151871 have potential to treat a variety of diseases and disorders including cancer, anemia of chronic disease, anemia of chronic inflammation, anemia of cancer, fibrodysplasisa ossificans progressive (FOP), neoplastic cutaneous disease, psoriasis, mycoses fungoides, benign prostatic hypertrophy, diabetes and related diseases such as diabetic retinopathy, retinal ischemia, and retinal neovascularization, hepatic cirrhosis, angiogenesis, cardiovascular disease such as atherosclerosis, immunological disease such as autoimmune disease, and renal disease. Cancer may include myeloproliferative disorders, lymphomas, or a solid tumor disorder. More particularly, cancers may include hematological cancers, such as acute myelogenous leukemia (AML) and chronic myelogenous leukemia (CML), lung cancer, NSCLC (non small cell lung cancer), oat cell cancer, bone cancer, pancreatic cancer, skin cancer, dermatofibrosarcoma protuberans, cancer of the head and neck, cutaneous or intraocular melanoma, uterine cancer, ovarian cancer, colorectal cancer, cancer of the anal region, stomach cancer, colon cancer, breast cancer, gynecologic tumors such as uterine sarcomas, carcinoma of the fallopian tubes, carcinoma of the endometrium, carcinoma of the cervix, carcinoma of the vagina, or carcinoma of the vulva, Hodgkin's Disease, hepatocellular cancer, cancer of the esophagus, cancer of the small intestine, cancer of the endocrine system such as cancer of the thyroid, pancreas, parathyroid, or adrenal glands, sarcomas of soft tissues, cancer of the urethra, cancer of the penis, testicular cancer, prostate cancer (particularly hormone-refractory), chronic or acute leukemia, solid tumors of childhood, hypereosinophilia, lymphocytic lymphomas, cancer of the bladder, cancer of the kidney or ureter, such as renal cell carcinoma or carcinoma of the renal pelvis, pediatric malignancy, neoplasms of the central nervous system, such as primary CNS lymphoma, spinal axis tumors, medulloblastoma, brain stem gliomas including diffuse intrinsic pontine glioma, or pituitary adenomas, and a pre-malignant syndrome such as Barrett's esophagus.

Patients diagnosed with one or more disease or disorder mediated by or associated with one or more of ALK2 (ACVR1), JAK2, and ALK5 would benefit from an oral solid dosage form to deliver the active pharmaceutical agent.

### SUMMARY OF THE DISCLOSURE

One embodiment of the present disclosure includes an oral solid pharmaceutical composition comprising
a. a compound of formula (I):

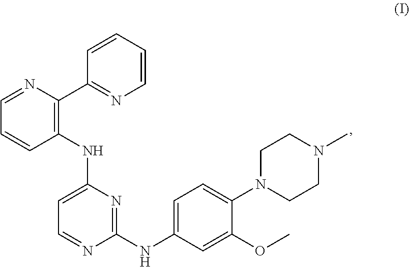 [SMILES: COc1cc(Nc2nccc(Nc3cccnc3-c3ccccn3)n2)ccc1N1CCN(C)CC1]

or a pharmaceutically acceptable salt thereof;
b. microcrystalline cellulose;
c. lactose;
d. croscarmellose; and
e. magnesium stearate.

In one aspect, the pharmaceutically acceptable salt is a hydrochloric acid salt. In one aspect, the pharmaceutical composition is a gelatin capsule. In one aspect, the gelatin capsule is (i) 5 mg, (ii) 25 mg, or (iii) 125 mg strength, based on free base weight. In one aspect, the gelatin capsule is (i) 30 mg, (ii) 60 mg, (iii) 90 mg, (iv) 120 mg, (v) 150 mg, (vi) 180 mg, (vii) 210 mg, (viii) 240 mg, (ix) 270 mg, or (x) 300 mg strength, based on free base weight. In one aspect, the amount of microcrystalline cellulose is from about 0% w/w to about 50% w/w. In one aspect, the amount of microcrystalline cellulose is from about 10% w/w to about 25% w/w. In one aspect, the amount of microcrystalline cellulose is from about 13% w/w to about 23% w/w. In one aspect, the amount of microcrystalline cellulose is from about 14% w/w to about 22% w/w. In one aspect, the amount of lactose is from about 10% w/w to about 80% w/w. In one aspect, the amount of lactose is from about 45% w/w to about 75% w/w. In one aspect, the amount of lactose is from about 46% w/w to about 72% w/w. In one aspect, the amount of lactose is from about 47% w/w to about 71% w/w. In one aspect, the amount of croscarmellose is from about 0.1% w/w to about 6.0% w/w. In one aspect, the amount of croscarmellose is about 3.0% w/w. In one aspect, the amount of magnesium stearate is from about 0.1% w/w to about 3.0% w/w. In one aspect, the amount of magnesium stearate is about 1.0% w/w.

One embodiment of the present disclosure includes an oral solid pharmaceutical composition comprising
a. a compound of formula (I):

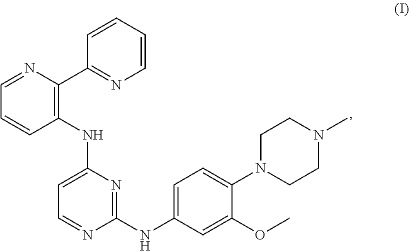 [SMILES: COc1cc(Nc2nccc(Nc3cccnc3-c3ccccn3)n2)ccc1N1CCN(C)CC1]

or a pharmaceutically acceptable salt thereof;
b. one or more diluent;
c. one or more disintegrant; and
d. one or more lubricant.

In one aspect, the composition comprises a total amount of diluent in an amount of about 10% w/w to about 80% w/w. In one aspect, the composition comprises two different diluents. In one aspect, one diluent is present in an amount of about 12% to about 25% and another diluent is present in amount of about 45% to about 75%. In one aspect, the one or more diluent is selected from microcrystalline cellulose, lactose, and combinations thereof. In one aspect, the one or more disintegrant is present in an amount of about 0.1% w/w to about 30.0% w/w. In one aspect, the one or more disintegrant is present in an amount of about 0.5% w/w to about 20.0% w/w. In one aspect, the one or more disintegrant is present in an amount of about 0.1% w/w to about 6.0% w/w. In one aspect, the amount of disintegrant is about 3.0% w/w. In one aspect, the disintegrant is croscarmellose sodium. In one aspect, the one or more lubricant is present in an amount of about 0.1% w/w to about 5.0% w/w. In one aspect, the one or more lubricant is present in an amount of about 0.5% w/w to about 3.0% w/w. In one aspect, the one or more lubricant is present in an amount of about 0.1% w/w to about 3.0% w/w. In one aspect, the amount of lubricant is about 1.0% w/w. In one aspect, the lubricant is magnesium stearate.

One embodiment of the present disclosure includes an oral solid pharmaceutical composition comprising:
a. about 5.4 mg of a compound of formula (I):

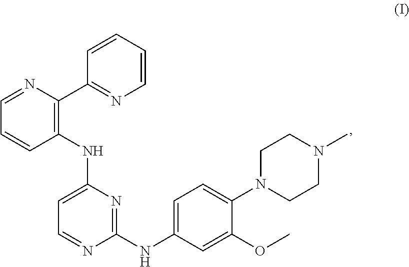 [SMILES: COc1cc(Nc2nccc(Nc3cccnc3-c3ccccn3)n2)ccc1N1CCN(C)CC1]

or a pharmaceutically acceptable salt thereof;
b. about 36.80 mg of microcrystalline cellulose;
c. about 121.00 mg of lactose;
d. about 5.10 mg of croscarmellose sodium; and
e. about 1.70 mg of magnesium stearate.

One embodiment of the present disclosure includes an oral solid pharmaceutical composition comprising:
a. about 27.00 mg of a compound of formula (I):

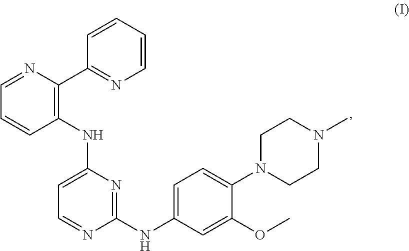 [SMILES: COc1cc(Nc2nccc(Nc3cccnc3-c3ccccn3)n2)ccc1N1CCN(C)CC1]

or a pharmaceutically acceptable salt thereof;
b. about 31.50 mg of microcrystalline cellulose;
c. about 104.70 mg of lactose;
d. about 5.10 mg of croscarmellose sodium; and
e. about 1.70 mg of magnesium stearate.

One embodiment of the present disclosure includes an oral solid pharmaceutical composition comprising:
a. about 135.00 mg of a compound of formula (I):

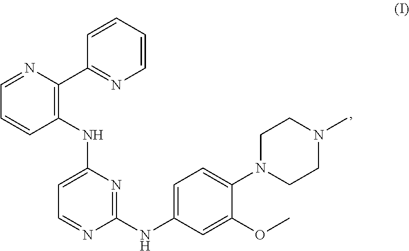 [SMILES: COc1cc(Nc2nccc(Nc3cccnc3-c3ccccn3)n2)ccc1N1CCN(C)CC1]

or a pharmaceutically acceptable salt thereof;
b. about 53.10 mg of microcrystalline cellulose;
c. about 176.70 mg of lactose;
d. about 11.40 mg of croscarmellose sodium; and
e. about 3.80 mg of magnesium stearate.

One embodiment of the present disclosure includes an oral solid pharmaceutical composition comprising:
a. about 3.18% w/w of a compound of formula (I):

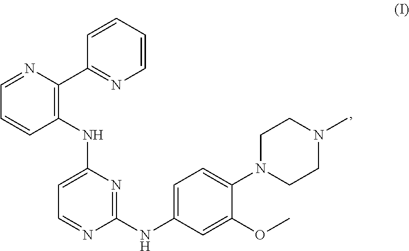 [SMILES: COc1cc(Nc2nccc(Nc3cccnc3-c3ccccn3)n2)ccc1N1CCN(C)CC1]

or a pharmaceutically acceptable salt thereof;
b. about 21.65% w/w of microcrystalline cellulose;
c. about 71.18% w/w of lactose;
d. about 3.00% w/w of croscarmellose sodium; and
e. about 1.00% w/w of magnesium stearate.

One embodiment of the present disclosure includes an oral solid pharmaceutical composition comprising:
a. about 15.88% w/w of a compound of formula (I):

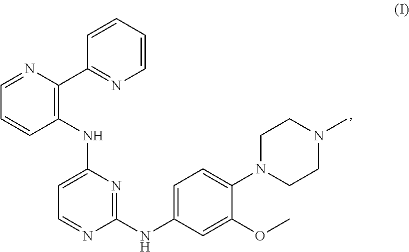 [SMILES: COc1cc(Nc2nccc(Nc3cccnc3-c3ccccn3)n2)ccc1N1CCN(C)CC1]

or a pharmaceutically acceptable salt thereof;
b. about 18.53% w/w of microcrystalline cellulose;
c. about 61.59% w/w of lactose;
d. about 3.00% w/w of croscarmellose sodium; and
e. about 1.00% w/w of magnesium stearate.

One embodiment of the present disclosure includes an oral solid pharmaceutical composition comprising:
a. about 35.53% w/w of a compound of formula (I):

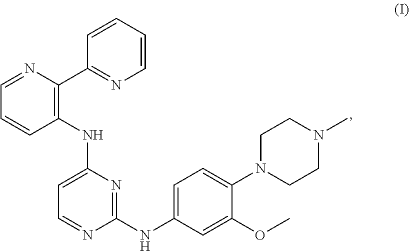 [SMILES: COc1cc(Nc2nccc(Nc3cccnc3-c3ccccn3)n2)ccc1N1CCN(C)CC1]

or a pharmaceutically acceptable salt thereof;
b. about 13.97% w/w of microcrystalline cellulose;
c. about 46.50% w/w of lactose;
d. about 3.00% w/w of croscarmellose sodium; and
e. about 1.00% w/w of magnesium stearate.

One embodiment of the present disclosure includes a method of treating a disease or disorder mediated by or associated with inhibition of one or more of ALK2 (ACVR1), JAK2, and ALK5 comprising administering a pharmaceutical composition of the present disclosure.

One embodiment of the present disclosure includes a composition for use in medicine comprising the composition of the present disclosure.

One embodiment of the present disclosure includes a composition of the present disclosure as a medicament for the treatment of a disease or disorder mediated by or associated with inhibition of one or more of ALK2 (ACVR1), JAK2, and ALK5.

One embodiment of the present disclosure includes use of a composition of the present disclosure for the treatment of a disease or disorder mediated by or associated with inhibition of one or more of ALK2 (ACVR1), JAK2, and ALK5.

One or more aspects and embodiments may be incorporated in a different embodiment although not specifically described. That is, all aspects and embodiments may be combined in any way or combination.

These and other aspects of the disclosure will be apparent upon reference to the following detailed description.

### BRIEF DESCRIPTION OF THE DRAWINGS

In the figures, identical reference numbers identify similar elements. The sizes and relative positions of elements in the figures are not necessarily drawn to scale and some of these elements are arbitrarily enlarged and positioned to improve figure legibility. Further, the particular shapes of the elements as drawn are not intended to convey any information regarding the actual shape of the particular elements, and have been solely selected for ease of recognition in the figures.

FIG. 1 is a graphical illustration of the dissolution profile of a capsule with a compound of formula (I) as the active pharmaceutical ingredient (“API”) at a 5 mg strength in pH 1.2 (0.1N HCl).

FIG. 2 is a graphical illustration of the dissolution profile of a capsule with a compound of formula (I) as the API at a 25 mg strength in pH 1.2 (0.1N HCl).

FIG. 3 is a graphical illustration of the dissolution profile of a capsule with a compound of formula (I) as the API at a 125 mg strength in pH 1.2 (0.1N HCl).

FIG. 4 is a graphical illustration of the dissolution profile of a capsule with a compound of formula (I) as the API at a 5 mg strength in pH 1.2 (0.1N HCl).

FIG. 5 is a graphical illustration of the dissolution profile of a capsule with a compound of formula (I) as the API at a 25 mg strength in pH 1.2 (0.1N HCl).

FIG. 6 is a graphical illustration of the dissolution profile of a capsule with a compound of formula (I) as the API at a 125 mg strength in pH 1.2 (0.1N HCl).

FIG. 7 is a flow diagram illustrating a manufacturing process for the compositions of the present disclosure.

FIG. 8 is a graphical illustration of the dissolution profiles of three (3) batches of capsules with a compound of formula (I) as the API at 5 mg, 25 mg, and 125 mg strengths, respectively, in pH 1.2 (0.1N HCl).

### DETAILED DESCRIPTION OF THE DISCLOSURE

Prior to setting forth this disclosure in more detail, it may be helpful to an understanding thereof to provide definitions of certain terms to be used herein. Additional definitions are set forth throughout this disclosure. In the following description, certain specific details are set forth in order to provide a thorough understanding of various embodiments of the invention. However, one skilled in the art will understand that the invention may be practiced without these details.

Unless the context requires otherwise, throughout the present specification and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the features, structures, or characteristics may be combined in any suitable manner in one or more embodiments.

The use of the words “optional” or “optionally” means that the subsequently described event or circumstances may or may not occur, and that the description includes instances where the event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable salt” includes both acid and base addition salts.

“Pharmaceutically acceptable acid addition salt” refers to those salts which are formed with inorganic acids such as hydrochloric acid, hydrobromic acid, sulfuric acid, nitric acid, phosphoric acid and the like, and organic acids such as acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid (e.g., L-(+)-tartaric acid), thiocyanic acid, p-toluenesulfonic acid, trifluoroacetic acid, undecylenic acid, and the like.

“Pharmaceutically acceptable base addition salt” refers to those salts which are prepared from addition of an inorganic base or an organic base to the free acid. Salts derived from inorganic bases include the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2 dimethylaminoethanol, 2 diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N ethylpiperidine, polyamine resins and the like. Preferred organic bases are isopropylamine, diethylamine, ethanolamine, trimethylamine, dicyclohexylamine, choline and caffeine.

In some embodiments, pharmaceutically acceptable salts include quaternary ammonium salts such as quaternary amine alkyl halide salts (e.g., methyl bromide).

Often crystallizations produce a solvate of the active agent of the disclosure. As used herein, the term “solvate” refers to an aggregate that comprises one or more molecules of an active agent of the disclosure with one or more molecules of solvent. The solvent may be water, in which case the solvate may be a hydrate. Alternatively, the solvent may be an organic solvent. Thus, the active agents of the present disclosure may exist as a hydrate, including a monohydrate, dihydrate, hemihydrate, sesquihydrate, trihydrate, tetrahydrate and the like, as well as the corresponding solvated forms. The active agent of the disclosure may be true solvates, while in other cases, the active agent of the disclosure may merely retain adventitious water or be a mixture of water plus some adventitious solvent.

The term “substantially” refers to a significant qualitative or quantitative extent. As an example, when used in the context to refer to a characterization of a compound, the term refers to an ability to identify a chemical substance based on material similarity with a referenced characterization method, such as, for example, XRPD, DSC, or TGA. Error ranges for such techniques, as are appreciated by those skilled in the art, are encompassed within the term “substantially.” Moreover, as used herein, “substantially pure,” when used in reference to a form, means a compound (e.g., a compound of formula (I)) having a purity greater than 90 weight %, including greater than 90, 91, 92, 93, 94, 95, 96, 97, 98, 98.5, 99, 99.1, 99.2, 99.3, 99.4, 99.5. 99.6, 99.7, 99.8, 99.9 weight %, and also including equal to 100 weight % of a compound of formula (I), based on the weight of the compound. The remaining material comprises other form(s) of the compound, and/or reaction impurities and/or processing impurities arising from its preparation. For example, a crystalline form of a compound of formula (I) may be deemed substantially pure in that it has a purity greater than 90 weight %, as measured by means that are at this time known and generally accepted in the art, where the remaining less than 10 weight % of material comprises other form(s) of a compound of formula (I) and/or reaction impurities and/or processing impurities. Another way to define substantially pure is following: . . . As used herein, the term “substantially pure” with reference to a polymorphic form means that the polymorphic form includes less than 10%, preferably less than 5%, more preferably less than 3%, most preferably less than 1% by weight of any other physical forms of the compound.

A “pharmaceutical composition” refers to a formulation of one or more therapeutic agents and a medium generally accepted in the art for the delivery of the biologically active agent to subjects, e.g., humans. Such a medium includes all pharmaceutically acceptable carriers, diluents, or excipients. “Pharmaceutically acceptable carrier, diluent, or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Effective amount” or “therapeutically effective amount” refers to that amount of a compound of the invention (e.g., a compound of formula (I)), which, when administered to a mammal, preferably a human, is sufficient to effect treatment, as defined below, of cancer in the mammal, preferably a human. The amount of a compound of the invention (e.g., a compound of formula (I)) which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure. In embodiments, an “effective amount” effects treatment (e.g., treats, prevents, inhibits, relieves, promotes, improves, increases, reduces, and the like) as measured by a statistically significant change in one or more indications, symptoms, signs, diagnostic tests, vital signs, and the like. In other embodiments, an “effective amount” suppresses, manages, or prevents a condition as measured by a lack of a statistically significant change in one or more indications, symptoms, signs, diagnostic tests, vital signs, and the like.

“Treating” or “treatment” as used herein covers the treatment of the disease or condition of interest in a mammal, preferably a human, having the disease or condition of interest, and includes:
(i) preventing the disease or condition from occurring in a mammal, when such mammal is predisposed to the condition but has not yet been diagnosed as having it;
(ii) inhibiting the disease or condition, i.e., arresting its development; (iii) relieving the disease or condition, i.e., causing regression of the disease or condition; or
(iv) relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

Therefore, “treating” or “treatment” as used herein, refers to the administration of a medication or medical care to a subject, such as a human, having a disease or condition of interest, e.g., a cancer, including: inhibiting the disease or condition, i.e., arresting its development; relieving the disease or condition, i.e., causing regression of the disease or condition; or relieving the symptoms resulting from the disease or condition, (e.g., pain, weight loss, cough, fatigue, weakness, etc.) without addressing the underlying disease or condition.

As used herein, the terms “disease,” “disorder,” and “condition” may be used interchangeably or may be different in that the particular malady or condition may not have a known causative agent (so that etiology has not yet been confirmed) and it is therefore not yet recognized as a disease but only as an undesirable condition or syndrome, wherein a more or less specific set of symptoms have been identified by clinicians.

“Subject” includes humans, domestic animals, such as laboratory animals (e.g., dogs, monkeys, rats, mice, etc.), household pets (e.g., cats, dogs, rabbits, etc.), and livestock (e.g., pigs, cattle, sheep, goats, horses, etc.), and non-domestic animals (e.g., bears, elephants, porcupines, etc.). In embodiments, the subject is a mammal. In embodiments, a subject is a human. The term “patient” may be used interchangeably with the term “subject.”

As used herein, “statistically significant” refers to a p value of 0.050 or less when calculated using the Students t-test and indicates that it is unlikely that a particular event or result being measured has arisen by chance.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the term “about” means±20%, ±10%, ±5% or ±1% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives.

Unless defined otherwise, all technical and scientific terms herein have the same meaning as commonly understood by one of ordinary skill in the art to which this disclosure belongs.

In the following description, certain specific details are set forth in order to provide a thorough understanding of various embodiments of this disclosure. However, one skilled in the art will understand that the disclosure may be practiced without these details.

Accordingly, the present disclosure provides compositions comprising an active agent (e.g., a compound of formula (I) or a pharmaceutically acceptable salt thereof) for use in the treatment of one or more disease or disorder mediated by or associated with ALK2 (ACVR1) or the Janus kinases (JAK) including JAK1, JAK2, JAK3, and JAKS. In a preferred embodiment, the active agent is formulated for oral administration. In various embodiments, the active agent is formulated as a tablet, capsule, such as a gelatin capsule. In some embodiments, the active agent is formulated with an excipient. In some embodiments, the gelatin capsules are formulated in 5 mg, 25 mg, or 125 mg strengths. In some embodiments, the capsules are formulated in 30 mg, 90 mg, or 120 mg strengths.

A preferred active agent is a compound of formula (I):

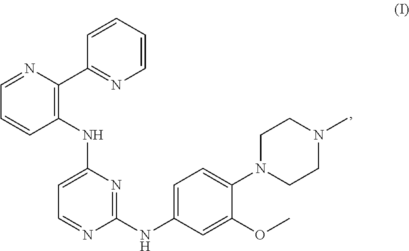 [SMILES: COc1cc(Nc2nccc(Nc3cccnc3-c3ccccn3)n2)ccc1N1CCN(C)CC1]

(i.e., N⁴-([2,2′-bipyridin]-3-yl)-N²-(3-methoxy-4-(4-methylpiperazin-1-yl)phenyl) pyrimidine-2,4-diamine), or a pharmaceutically acceptable salt or prodrug thereof. The compound may be prepared according to any number of methods known in the art, including methods described in US 2016/0214944, which is hereby incorporated by reference.

In some embodiments, the pharmaceutically acceptable salt in the pharmaceutical compositions of this disclosure comprises hydrochloric acid (HCl) salt, fumarate, sulfate, phosphate, succinate, tartrate, Hippurate, maleate, and/or Malate salts.

In one embodiment, the pharmaceutically acceptable salt is HCl or fumarate salt.

In one embodiment, the pharmaceutically acceptable salt is HCl salt.

In another embodiment, the pharmaceutically acceptable salt is fumarate salt.

In some embodiments, the compound of formula (I) may be present in the pharmaceutical compositions as a crystalline (freebase) solid form or a crystalline salt form.

In some embodiments, the crystalline salt form is HCl salt Form A, HCl salt Form B, HCl salt Form C, HCl salt Form D, HCl salt Form E, HCl salt Form F, or HCl salt Form G.

In some embodiments, the crystalline salt form is fumarate Form A, sulfate Form A, phosphate Form A, succinate Form A, tartrate Form A, hippurate Form A, maleate Form A, maleate Form B, maleate Form C, or malate Form A. The crystalline solid form and crystalline salt forms can be prepared according to the procedure disclosed in WO2020/023910, the content of which is incorporated herein by reference in its entirety for all purposes.

In one embodiment, the crystalline salt is HCl crystalline salt of the compound of formula (I). In another embodiment, the HCl crystalline salt comprises Form A. In one embodiment, the HCl crystalline salt form consists essentially of Form A. In another embodiment, the Form A is substantially free from impurities.

In one embodiment, the crystalline salt is Form A of N⁴-(2,2′-bipyridin-3-yl)-N²-(3-methoxy-4-(4-methylpiperazin-1-yl)phenyl)pyrimidine-2,4-diamine anhydrous hydrochloric acid salt.

In another embodiment, Form A of N⁴-(2,2′-bipyridin-3-yl)-N²-(3-methoxy-4-(4-methylpiperazin-1-yl)phenyl)pyrimidine-2,4-diamine anhydrous hydrochloric acid salt is characterized by an x-ray diffraction pattern (XRPD) comprising one or more 20 values selected from 13.53, 16.14, 17.67, 18.38, 24.96, and 28.18. In one embodiment, Form A is further characterized by 20 values selected from 6.71, 19.25, 23.98, and 29.60. In one embodiment, the 20 values are within +/−0.2 2θ. The table below provides the XRPD pattern of the HCl salt Form A of the compound of formula (I).

| Pos. | Height | Area | d-spacing | Rel. Int. |
|---|---|---|---|---|
| [°2θ] | [cts] | [cts °2θ] | [Å] | [%] |
| 3.9059 | 298.79 | 37.70 | 22.62205 | 2.85 |
| 6.7125 | 2201.43 | 222.23 | 13.16854 | 20.97 |
| 8.8145 | 454.58 | 51.63 | 10.03229 | 4.33 |
| 10.1413 | 1672.42 | 211.04 | 8.72261 | 15.93 |
| 11.0558 | 561.23 | 70.82 | 8.00302 | 5.35 |
| 12.7048 | 1080.46 | 136.34 | 6.96775 | 10.29 |
| 13.5347 | 6642.71 | 922.04 | 6.54234 | 63.28 |
| 13.8769 | 1131.77 | 142.81 | 6.38178 | 10.78 |
| 14.1755 | 1620.14 | 224.88 | 6.24800 | 15.43 |
| 15.1840 | 3546.33 | 537.00 | 5.83520 | 33.78 |
| 15.8491 | 5585.02 | 775.23 | 5.59182 | 53.21 |
| 16.1455 | 8599.25 | 1193.62 | 5.48981 | 81.92 |
| 17.2245 | 162.88 | 26.72 | 5.14828 | 1.55 |
| 17.6770 | 2882.25 | 436.44 | 5.01749 | 27.46 |
| 18.3807 | 2578.71 | 390.48 | 4.82696 | 24.57 |
| 19.2499 | 1100.51 | 166.64 | 4.61093 | 10.48 |
| 19.7721 | 9580.62 | 1450.74 | 4.49032 | 91.27 |
| 20.2054 | 5072.51 | 896.12 | 4.39499 | 48.32 |
| 20.8140 | 3168.56 | 399.83 | 4.26782 | 30.19 |
| 20.9432 | 2000.42 | 227.18 | 4.24178 | 19.06 |
| 22.0018 | 366.67 | 64.72 | 4.04003 | 3.49 |
| 22.6847 | 604.86 | 76.32 | 3.91994 | 5.76 |
| 23.9816 | 1024.54 | 181.00 | 3.71080 | 9.76 |
| 24.4538 | 531.66 | 80.51 | 3.64021 | 5.06 |
| 24.9644 | 2159.45 | 326.99 | 3.56690 | 20.57 |
| 25.5118 | 4541.08 | 802.23 | 3.49160 | 43.26 |
| 26.1922 | 763.31 | 86.69 | 3.40242 | 7.27 |
| 26.7501 | 1326.52 | 200.87 | 3.33272 | 12.64 |
| 27.2385 | 10497.03 | 1854.42 | 3.27406 | 100.0 |
| 28.1817 | 2992.14 | 528.60 | 3.16659 | 28.50 |
| 28.5514 | 789.98 | 109.65 | 3.12642 | 7.53 |
| 28.8497 | 599.52 | 90.78 | 3.09477 | 5.71 |
| 29.6008 | 1378.42 | 260.91 | 3.01793 | 13.13 |
| 30.4479 | 633.83 | 103.97 | 2.93586 | 6.04 |
| 31.0671 | 347.19 | 52.57 | 2.87875 | 3.31 |
| 31.9977 | 365.28 | 92.19 | 2.79712 | 3.48 |
| 32.4347 | 376.35 | 80.73 | 2.76042 | 3.59 |
| 33.3026 | 174.88 | 26.48 | 2.69045 | 1.67 |
| 33.6159 | 293.68 | 44.47 | 2.66608 | 2.80 |
| 34.0326 | 123.70 | 18.73 | 2.63439 | 1.18 |
| 34.7780 | 211.12 | 42.62 | 2.57961 | 2.01 |
| 35.5705 | 123.93 | 25.02 | 2.52394 | 1.18 |
| 36.6319 | 310.89 | 86.31 | 2.45321 | 2.96 |
| 37.4707 | 246.78 | 43.60 | 2.40020 | 2.35 |
| 38.1695 | 235.29 | 71.26 | 2.35785 | 2.24 |
| 40.1011 | 135.41 | 34.18 | 2.24862 | 1.29 |
| 40.7471 | 678.30 | 77.03 | 2.21445 | 6.46 |
| 41.2836 | 367.58 | 74.21 | 2.18690 | 3.50 |
| 41.9985 | 284.08 | 64.53 | 2.15132 | 2.71 |
| 43.6795 | 124.43 | 31.40 | 2.07234 | 1.19 |
| 44.5269 | 137.92 | 34.81 | 2.03485 | 1.31 |
| 45.4240 | 143.20 | 36.14 | 1.99673 | 1.36 |
| 46.4339 | 166.33 | 58.77 | 1.95563 | 1.58 |
| 47.3800 | 176.53 | 53.46 | 1.91877 | 1.68 |

In one embodiment, Form A of N⁴-(2,2′-bipyridin-3-yl)-N²-(3-methoxy-4-(4-methylpiperazin-1-yl)phenyl)pyrimidine-2,4-diamine anhydrous hydrochloric acid salt is characterized by an endotherm at one or more of 196.2° C., 214.8° C., and 274.0° C.

In one embodiment, Form A is characterized by a peak endotherm at one or more of 198.9° C., 218.0° C., and 275.9° C. In one embodiment, the form is further characterized by an onset temperature of 274.0° C. In one embodiment, the form is further characterized by weight loss of 1.7% up to 150° C.

### Pharmaceutical Composition

A pharmaceutical composition for use in embodiments of the disclosure may include various materials, which modify the physical form of a solid dosage unit. For example, the composition may include materials that form a coating shell around the active agent. The materials that form the coating shell are typically inert, and may be selected from, for example, sugar, shellac, and other enteric coating agents. Alternatively, the active ingredient(s) may be encased in a gelatin capsule.

In some embodiments, the concentration an active agent provided in the pharmaceutical compositions is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v. In embodiments, the concentration and/or weight of an active agent is based on the free base weight.

In some embodiments, the concentration of an active agent provided in the pharmaceutical compositions is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of an active agent provided in the pharmaceutical compositions is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12%, about 1% to about 10% w/w, w/v or v/v.

In some embodiments, the concentration of an active agent provided in the pharmaceutical compositions is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, or about 0.1% to about 0.9% w/w, w/v or v/v.

In some embodiments, the present disclosure provides an oral solid pharmaceutical composition comprising:
a compound of formula (I):

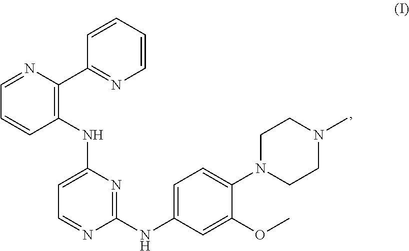 [SMILES: COc1cc(Nc2nccc(Nc3cccnc3-c3ccccn3)n2)ccc1N1CCN(C)CC1]

or a pharmaceutically acceptable salt thereof;
one or more diluent;
one or more disintegrant; and
one or more lubricant; wherein the pharmaceutically acceptable salt is hydrochloric acid salt.

In some embodiments, the pharmaceutically acceptable salt is a hydrochloric acid crystalline salt. In some embodiments, the pharmaceutical composition comprises Form A of the hydrochloric acid crystalline salt of the compound of formula (I).

In some embodiments, the composition comprises a total amount of diluent in an amount of about 10% w/w to about 80% w/w. In one embodiment, the composition comprises two different diluents. In one embodiment, one diluent is present in an amount of about 12% to about 25% and another diluent is present in amount of about 45% to about 75%. In one embodiment, the one or more diluent is selected from microcrystalline cellulose, lactose, and combinations thereof.

In some embodiments, the one or more disintegrant is present in an amount of about 0.1% w/w to about 30.0% w/w. In one embodiment, the one or more disintegrant is present in an amount of about 0.5% w/w to about 20.0% w/w. In one embodiment, the one or more disintegrant is present in an amount of about 0.1% w/w to about 6.0% w/w. In one embodiment, the amount of disintegrant is about 3.0% w/w. In one embodiment, the disintegrant is croscarmellose sodium.

In some embodiments, the one or more lubricant is present in an amount of about 0.1% w/w to about 5.0% w/w. In one embodiment, the one or more lubricant is present in an amount of about 0.5% w/w to about 3.0% w/w. In one embodiment, the one or more lubricant is present in an amount of about 0.1% w/w to about 3.0% w/w. In one embodiment, the amount of lubricant is about 1.0% w/w. In one embodiment, the lubricant is magnesium stearate.

In some embodiments, the present disclosure includes an oral solid pharmaceutical composition comprising:
about 3 mg to about 350 mg of a compound of formula (I), or a pharmaceutically acceptable salt thereof;
one or more diluent;
one or more disintegrant; and
one or more lubricant.

One embodiment of the present disclosure includes an oral solid pharmaceutical composition comprising:
about 3 mg to about 150 mg of a compound of formula (I), or a pharmaceutically acceptable salt thereof;
about 30 mg to 260 mg of one or more diluent;
about 3 mg to about 13 mg of one or more disintegrant; and
about 1 mg to about 5 mg of one or more lubricant.

One embodiment of the present disclosure includes an oral solid pharmaceutical composition comprising:
about 1-50% w/w of a compound of formula (I), or a pharmaceutically acceptable salt thereof;
about 10-95% w/w of one or more diluent;
about 0.1-6.0% w/w of one or more disintegrant; and
about 0.1-3.0% w/w of one or more lubricant.

One embodiment of the present disclosure includes an oral solid pharmaceutical composition comprising:
about 2-38% w/w of a compound of formula (I), or a pharmaceutically acceptable salt thereof;
about 10-80% w/w of one or more diluent;
about 2-4% w/w of one or more disintegrant; and
about 0.7-1.3% w/w of one or more lubricant.

In some embodiments, the present disclosure provides an oral solid pharmaceutical composition comprising:
a compound of formula (I), or a pharmaceutically acceptable salt thereof;
microcrystalline cellulose;
lactose;
croscarmellose; and
magnesium stearate,
   wherein the pharmaceutical composition is a gelatin capsule or tablet.

In one embodiment, the pharmaceutically acceptable salt in the gelatin capsule or tablet is a hydrochloric acid salt. In one embodiment, the hydrochloric acid salt is a crystalline salt. In one embodiment, the crystalline salt comprises Form A of the hydrochloric acid crystalline salt. In one embodiment, the pharmaceutical composition is a gelatin capsule.

In one embodiment, the gelatin capsule is (i) 5 mg, (ii) 25 mg, or (iii) 125 mg strength, based on free base weight. In one embodiment, the gelatin capsule is (i) 30 mg, (ii) 60 mg, (iii) 90 mg, (iv) 120 mg, (v) 150 mg, (vi) 180 mg, (vii) 210 mg, (viii) 240 mg, (ix) 270 mg, or (x) 300 mg strength, based on free base weight.

In one embodiment, the gelatin capsule comprises microcrystalline cellulose in an amount from about 0% w/w to about 50% w/w. In one embodiment, the amount of microcrystalline cellulose is from about 10% w/w to about 25% w/w. In one embodiment, the amount of microcrystalline cellulose is from about 13% w/w to about 23% w/w. In one embodiment, the amount of microcrystalline cellulose is from about 14% w/w to about 22% w/w. In one embodiment, the amount of lactose is from about 10% w/w to about 80% w/w.

In one embodiment, the gelatin capsule comprises lactose in an amount from about 45% w/w to about 75% w/w. In one embodiment, the amount of lactose is from about 46% w/w to about 72% w/w. In one embodiment, the amount of lactose is from about 47% w/w to about 71% w/w.

In one embodiment, the gelatin capsule comprises croscarmellose in an amount from about 0.1% w/w to about 6.0% w/w. In one embodiment, the amount of croscarmellose is about 3.0% w/w.

In one embodiment, the gelatin capsule comprises magnesium stearate in an amount from about 0.1% w/w to about 3.0% w/w. In one embodiment, the amount of magnesium stearate is about 1.0% w/w.

In one embodiment, the present disclosure includes an oral solid pharmaceutical composition comprising:
about 3 mg to about 150 mg of a compound of formula (I), or a pharmaceutically acceptable salt thereof;
microcrystalline cellulose;
lactose;
croscarmellose sodium; and
magnesium stearate.

One embodiment of the present disclosure includes an oral solid pharmaceutical composition comprising:
about 3 mg to about 150 mg of a compound of formula (I), or a pharmaceutically acceptable salt thereof;
about 30 mg to about 60 mg of microcrystalline cellulose;
about 100 mg to about 200 mg of lactose;
about 3 mg to about 13 mg of croscarmellose sodium; and
about 1 mg to about 5 mg of magnesium stearate.

One embodiment of the present disclosure includes an oral solid pharmaceutical composition comprising:
about 3 mg to about 30 mg of a compound of formula (I), or a pharmaceutically acceptable salt thereof;
about 30 mg to about 60 mg of microcrystalline cellulose;
about 100 mg to about 200 mg of lactose;
about 3 mg to about 13 mg of croscarmellose sodium; and
about 1 mg to about 5 mg of magnesium stearate.

One embodiment of the present disclosure includes an oral solid pharmaceutical composition comprising:
about 5 mg to 6 mg of a compound of formula (I), or a pharmaceutically acceptable salt thereof;
about 33 mg to 39 mg microcrystalline cellulose;
about 115 mg to 125 mg lactose;
about 4 mg to 6 mg croscarmellose sodium; and
about 1.4 mg to 2 mg magnesium stearate.

One embodiment of the present disclosure includes an oral solid pharmaceutical composition comprising:
about 5.4 mg of a compound of formula (I), or a pharmaceutically acceptable salt thereof;
about 33 mg to 39 mg microcrystalline cellulose;
about 118 mg to 124 mg lactose;
about 4 mg to 6 mg croscarmellose sodium; and
about 1.4 mg to 2 mg magnesium stearate.

One embodiment of the present disclosure includes an oral solid pharmaceutical composition comprising:
about 25.00 mg to 30 mg of a compound of formula (I), or a pharmaceutically acceptable salt thereof;
about 28 mg to 35 mg of microcrystalline cellulose;
about 100 mg to 110 mg of lactose;
about 4 mg to 6 mg of croscarmellose sodium; and
about 1.4 mg to 2 mg of magnesium stearate.

One embodiment of the present disclosure includes an oral solid pharmaceutical composition comprising:
about 27.00 mg of a compound of formula (I), or a pharmaceutically acceptable salt thereof;
about 28 mg to 35 mg of microcrystalline cellulose;
about 100 mg to 110 mg of lactose;
about 4 mg to 6 mg of croscarmellose sodium; and
about 1.4 mg to 2 mg of magnesium stearate.

One embodiment of the present disclosure includes an oral solid pharmaceutical composition comprising:
about 125.00 mg to 140 mg of a compound of formula (I), or a pharmaceutically acceptable salt thereof;
about 48 mg to 58 mg of microcrystalline cellulose;
about 170 mg to 180 mg of lactose;
about 8 mg to 14 mg of croscarmellose sodium; and
about 3.3 mg to 4.3 mg of magnesium stearate.

One embodiment of the present disclosure includes an oral solid pharmaceutical composition comprising:
about 135.00 mg of a compound of formula (I), or a pharmaceutically acceptable salt thereof;
about 48 mg to 58 mg of microcrystalline cellulose;
about 170 mg to 180 mg of lactose;
about 8 mg to 14 mg of croscarmellose sodium; and
about 3.3 mg to 4.3 mg of magnesium stearate.

One embodiment of the present disclosure includes an oral solid pharmaceutical composition comprising:
about 5.4 mg of a compound of formula (I), or a pharmaceutically acceptable salt thereof;
about 36.80 mg of microcrystalline cellulose;
about 121.00 mg of lactose;
about 5.10 mg of croscarmellose sodium; and
about 1.70 mg of magnesium stearate.

One embodiment of the present disclosure includes an oral solid pharmaceutical composition comprising:
about 27.00 mg of a compound of formula (I), or a pharmaceutically acceptable salt thereof;
about 31.50 mg of microcrystalline cellulose;
about 104.70 mg of lactose;
about 5.10 mg of croscarmellose sodium; and
about 1.70 mg of magnesium stearate.

One embodiment of the present disclosure includes an oral solid pharmaceutical composition comprising:
about 135.00 mg of a compound of formula (I), or a pharmaceutically acceptable salt thereof;
about 53.10 mg of microcrystalline cellulose;
about 176.70 mg of lactose;
about 11.40 mg of croscarmellose sodium; and
about 3.80 mg of magnesium stearate.

One embodiment of the present disclosure includes an oral solid pharmaceutical composition comprising:
about 2-38% w/w of a compound of formula (I), or a pharmaceutically acceptable salt thereof;
about 12-25% w/w of microcrystalline cellulose;
about 45-75% w/w of lactose;
about 2-4% w/w of croscarmellose sodium; and
about 0.7-1.3% w/w of magnesium stearate.

One embodiment of the present disclosure includes an oral solid pharmaceutical composition comprising:
about 2-38% w/w of a compound of formula (I), or a pharmaceutically acceptable salt thereof;
about 12-25% w/w of microcrystalline cellulose;
about 45-75% w/w of lactose;
about 3% w/w of croscarmellose sodium; and
about 1% w/w of magnesium stearate.

One embodiment of the present disclosure includes an oral solid pharmaceutical composition comprising:
about 3-3.3% w/w of a compound of formula (I), or a pharmaceutically acceptable salt thereof;
about 20-23% w/w of microcrystalline cellulose;
about 70-73% w/w of lactose;
about 3.00% w/w of croscarmellose sodium; and
about 1.00% w/w of magnesium stearate.

One embodiment of the present disclosure includes an oral solid pharmaceutical composition comprising:
about 3.18% w/w of a compound of formula (I), or a pharmaceutically acceptable salt thereof;
about 20-23% w/w of microcrystalline cellulose;
about 70-73% w/w of lactose;
about 3.00% w/w of croscarmellose sodium; and
about 1.00% w/w of magnesium stearate.

One embodiment of the present disclosure includes an oral solid pharmaceutical composition comprising:
about 14-17% w/w of a compound of formula (I), or a pharmaceutically acceptable salt thereof;
about 17-20% w/w of microcrystalline cellulose;
about 60-64% w/w of lactose;
about 3.00% w/w of croscarmellose sodium; and
about 1.00% w/w of magnesium stearate.

One embodiment of the present disclosure includes an oral solid pharmaceutical composition comprising:
about 15.88% w/w of a compound of formula (I), or a pharmaceutically acceptable salt thereof;
about 17-20% w/w of microcrystalline cellulose;
about 60-64% w/w of lactose;
about 3.00% w/w of croscarmellose sodium; and
about 1.00% w/w of magnesium stearate.

One embodiment of the present disclosure includes an oral solid pharmaceutical composition comprising:
about 34-37% w/w of a compound of formula (I), or a pharmaceutically acceptable salt thereof;
about 12-15% w/w of microcrystalline cellulose;
about 45-48% w/w of lactose;
about 3.00% w/w of croscarmellose sodium; and
about 1.00% w/w of magnesium stearate.

One embodiment of the present disclosure includes an oral solid pharmaceutical composition comprising:
about 35.53% w/w of a compound of formula (I), or a pharmaceutically acceptable salt thereof;
about 12-15% w/w of microcrystalline cellulose;
about 45-48% w/w of lactose;
about 3.00% w/w of croscarmellose sodium; and
about 1.00% w/w of magnesium stearate.

One embodiment of the present disclosure includes an oral solid pharmaceutical composition comprising:
about 3.18% w/w of a compound of formula (I), or a pharmaceutically acceptable salt thereof;
about 21.65% w/w of microcrystalline cellulose;
about 71.18% w/w of lactose;
about 3.00% w/w of croscarmellose sodium; and
about 1.00% w/w of magnesium stearate.

One embodiment of the present disclosure includes an oral solid pharmaceutical composition comprising:
about 15.88% w/w of a compound of formula (I), or a pharmaceutically acceptable salt thereof;
about 18.53% w/w of microcrystalline cellulose;
about 61.59% w/w of lactose;
about 3.00% w/w of croscarmellose sodium; and
about 1.00% w/w of magnesium stearate.

One embodiment of the present disclosure includes an oral solid pharmaceutical composition comprising:
about 35.53% w/w of a compound of formula (I), or a pharmaceutically acceptable salt thereof;
about 13.97% w/w of microcrystalline cellulose;
about 46.50% w/w of lactose;
about 3.00% w/w of croscarmellose sodium; and
about 1.00% w/w of magnesium stearate.

In one embodiment, the pharmaceutically acceptable salt in the above oral solid pharmaceutical compositions is a hydrochloric acid salt. In one embodiment, the pharmaceutically acceptable salt is a hydrochloric acid crystalline salt. In one aspect, the pharmaceutical composition comprises Form A of the hydrochloric acid crystalline salt.

One embodiment of the present disclosure includes an oral solid pharmaceutical composition comprising:
about 5 mg to 6 mg of hydrochloric acid salt of the compound of formula (I);
about 33 mg to 39 mg microcrystalline cellulose;
about 118 mg to 124 mg lactose;
about 4 mg to 6 mg croscarmellose sodium; and
about 1.4 mg to 2 mg magnesium stearate.

One embodiment of the present disclosure includes an oral solid pharmaceutical composition comprising:
about 5.4 mg of hydrochloric acid salt of the compound of formula (I);
about 33 mg to 39 mg microcrystalline cellulose;
about 118 mg to 124 mg lactose;
about 4 mg to 6 mg croscarmellose sodium; and
about 1.4 mg to 2 mg magnesium stearate.

One embodiment of the present disclosure includes an oral solid pharmaceutical composition comprising:
about 25.00 mg to 30 mg of hydrochloric acid salt of the compound of formula (I);
about 28 mg to 35 mg of microcrystalline cellulose;
about 100 mg to 110 mg of lactose;
about 4 mg to 6 mg of croscarmellose sodium; and
about 1.4 mg to 2 mg of magnesium stearate.

One embodiment of the present disclosure includes an oral solid pharmaceutical composition comprising:
about 27.00 mg of hydrochloric acid salt of the compound of formula (I); about 28 mg to 35 mg of microcrystalline cellulose;
about 100 mg to 110 mg of lactose;
about 4 mg to 6 mg of croscarmellose sodium; and about 1.4 mg to 2 mg of magnesium stearate.

One embodiment of the present disclosure includes an oral solid pharmaceutical composition comprising:
about 125.00 mg to 140 mg of hydrochloric acid salt of the compound of formula (I);
about 48 mg to 58 mg of microcrystalline cellulose;
about 170 mg to 180 mg of lactose;
about 8 mg to 14 mg of croscarmellose sodium; and
about 3.3 mg to 4.3 mg of magnesium stearate.

One embodiment of the present disclosure includes an oral solid pharmaceutical composition comprising:
about 135.00 mg of hydrochloric acid salt of the compound of formula (I);
about 48 mg to 58 mg of microcrystalline cellulose;
about 170 mg to 180 mg of lactose;
about 8 mg to 14 mg of croscarmellose sodium; and
about 3.3 mg to 4.3 mg of magnesium stearate.

One embodiment of the present disclosure includes an oral solid pharmaceutical composition comprising:
about 5.4 mg of hydrochloric acid salt of the compound of formula (I);
about 36.80 mg of microcrystalline cellulose;
about 121.00 mg of lactose;
about 5.10 mg of croscarmellose sodium; and
about 1.70 mg of magnesium stearate.

One embodiment of the present disclosure includes an oral solid pharmaceutical composition comprising:
about 27.00 mg of hydrochloric acid salt of the compound of formula (I);
about 31.50 mg of microcrystalline cellulose;
about 104.70 mg of lactose;
about 5.10 mg of croscarmellose sodium; and
about 1.70 mg of magnesium stearate.

One embodiment of the present disclosure includes an oral solid pharmaceutical composition comprising:
about 135.00 mg of hydrochloric acid salt of the compound of formula (I);
about 53.10 mg of microcrystalline cellulose;
about 176.70 mg of lactose;
about 11.40 mg of croscarmellose sodium; and
about 3.80 mg of magnesium stearate.

One embodiment of the present disclosure includes an oral solid pharmaceutical composition comprising:
about 3-3.3% w/w of hydrochloric acid salt of the compound of formula (I);
about 20-23% w/w of microcrystalline cellulose;
about 70-73% w/w of lactose;
about 3.00% w/w of croscarmellose sodium; and
about 1.00% w/w of magnesium stearate.

One embodiment of the present disclosure includes an oral solid pharmaceutical composition comprising:
about 3.18% w/w of hydrochloric acid salt of the compound of formula (I);
about 20-23% w/w of microcrystalline cellulose;
about 70-73% w/w of lactose;
about 3.00% w/w of croscarmellose sodium; and
about 1.00% w/w of magnesium stearate.

One embodiment of the present disclosure includes an oral solid pharmaceutical composition comprising:
about 14-17% w/w of hydrochloric acid salt of the compound of formula (I);
about 17-20% w/w of microcrystalline cellulose;
about 60-64% w/w of lactose;
about 3.00% w/w of croscarmellose sodium; and
about 1.00% w/w of magnesium stearate.

One embodiment of the present disclosure includes an oral solid pharmaceutical composition comprising:
about 15.88% w/w of hydrochloric acid salt of the compound of formula (I);
about 17-20% w/w of microcrystalline cellulose;
about 60-64% w/w of lactose;
about 3.00% w/w of croscarmellose sodium; and
about 1.00% w/w of magnesium stearate.

One embodiment of the present disclosure includes an oral solid pharmaceutical composition comprising:
about 34-37% w/w of hydrochloric acid salt of the compound of formula (I);
about 12-15% w/w of microcrystalline cellulose;
about 45-48% w/w of lactose;
about 3.00% w/w of croscarmellose sodium; and
about 1.00% w/w of magnesium stearate.

One embodiment of the present disclosure includes an oral solid pharmaceutical composition comprising:
about 35.53% w/w of hydrochloric acid salt of the compound of formula (I);
about 12-15% w/w of microcrystalline cellulose;
about 45-48% w/w of lactose;
about 3.00% w/w of croscarmellose sodium; and
about 1.00% w/w of magnesium stearate.

One embodiment of the present disclosure includes an oral solid pharmaceutical composition comprising:
about 3.18% w/w of hydrochloric acid salt of the compound of formula (I);
about 21.65% w/w of microcrystalline cellulose;
about 71.18% w/w of lactose;
about 3.00% w/w of croscarmellose sodium; and
about 1.00% w/w of magnesium stearate.

One embodiment of the present disclosure includes an oral solid pharmaceutical composition comprising:
about 15.88% w/w of hydrochloric acid salt of the compound of formula (I);
about 18.53% w/w of microcrystalline cellulose;
about 61.59% w/w of lactose;
about 3.00% w/w of croscarmellose sodium; and
about 1.00% w/w of magnesium stearate.

One embodiment of the present disclosure includes an oral solid pharmaceutical composition comprising:
about 35.53% w/w of hydrochloric acid salt of the compound of formula (I);
about 13.97% w/w of microcrystalline cellulose;
about 46.50% w/w of lactose;
about 3.00% w/w of croscarmellose sodium; and
about 1.00% w/w of magnesium stearate.

In some embodiments, the pharmaceutical composition comprising the compound of formula (I) is stable upon storage in an open or closed container at about 25 degrees Celsius and about 60 percent relative humidity for a period of at least about 1-18 months, about 18-24 months, about 24-30 months, or about 36 months.

In another embodiment, the pharmaceutical composition comprising the compound of formula (I) is stable upon storage in an open or closed container at about 25 degrees Celsius and about 60 percent relative humidity for at least 24 months with a total degradation product of no more than 0.1%.

In another embodiment, the pharmaceutical composition comprising the compound of formula (I) is stable upon storage in an open or closed container at about 40 degrees Celsius and about 75 percent relative humidity for a period of at least about 1-12 months.

In another embodiment, the pharmaceutical composition comprising the compound of formula (I) is stable upon storage in an open or closed container at about 40 degrees Celsius and about 75 percent relative humidity for at least 6 months with a total degradation product of no more than 0.1%.

In some embodiments, the pharmaceutical composition comprising the compound of formula (I) exhibits excellent dissolution properties. In one embodiment, the pharmaceutical composition comprising compound of formula (I) exhibits greater than 80% dissolution at 5 minutes. In another embodiment, the pharmaceutical composition comprising compound of formula (I) exhibits greater than 90% dissolution at 10 minutes. In another embodiment, the pharmaceutical composition comprising compound of formula (I) exhibits greater than 95% dissolution at 15 minutes.

In another embodiment, the pharmaceutical composition comprising the compound of formula (I) exhibits greater than 95% dissolution at 30 minutes after storage in an open or closed container at about 40 degrees Celsius and about 75 percent relative humidity for a period of at least about 1 month, 2 months, 3 months, 4 months, 5 months, or 6 months.

In another embodiment, the pharmaceutical composition comprising compound of formula (I) exhibits greater than 95% dissolution at 30 minutes after storage in an open or closed container at about 40 degrees Celsius and about 75 percent relative humidity for a period of at least 6 months.

In another embodiment, the pharmaceutical composition comprising the compound of formula (I) exhibits greater than 95% dissolution at 30 minutes after storage in an open or closed container at about 25 degrees Celsius and about 60 percent relative humidity for a period of at least about 1-12 months, about 12-18 months, or 24 months.

In another embodiment, the pharmaceutical composition comprising compound of formula (I) exhibits greater than 95% dissolution at 30 minutes after storage in an open or closed container at about 25 degrees Celsius and about 60 percent relative humidity for a period of at least 24 months.

In another embodiment, the pharmaceutical composition comprising compound of formula (I) exhibits greater than 97% dissolution at 30 minutes after storage in an open or closed container at about 25 degrees Celsius and about 60 percent relative humidity for a period of at least 24 months.

As a solid composition for oral administration, the pharmaceutical composition of the present disclosure may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as croscarmellose sodium, alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. The compositions may be uncoated or they may be coated by known techniques to delay disintegration and absorption in the gastrointestinal tract and thereby provide a sustained action over a longer period.

A pharmaceutical composition for use in embodiments of the disclosure may include various materials, which modify the physical form of a solid dosage unit. For example, the composition may include materials that form a coating shell around the active agent. The materials that form the coating shell are typically inert, and may be selected from, for example, sugar, shellac, and other enteric coating agents. Alternatively, the active ingredient(s) may be encased in a gelatin capsule.

Formulations for oral use may be hard gelatin capsules, wherein the active ingredient is mixed with an inert solid diluent, for example, calcium carbonate, calcium phosphate or kaolin. Capsules may also be soft gelatin capsules, wherein the active ingredient is mixed with water or miscible solvents such as propylene glycol, PEGs and ethanol, or an oil medium, for example, peanut oil, liquid paraffin, or olive oil.

### Medical Use and Method of Treatment

The present disclosure provides compositions comprising the compound of formula (I) or a pharmaceutically acceptable salt thereof for use in the treatment of one or more diseases or disorders mediated by or associated with ALK2 (ACVR1) or JAK. The present disclosure also provides compositions comprising the compound of formula (I) or a pharmaceutically acceptable salt thereof for use in the treatment for TGFβ type I receptor kinase (ALK5) mediated disorders or diseases (e.g., anemia, myelodysplastic syndrome (MDS) and anemia of chronic disease (ACD)) in a subject.

In some embodiments, the present disclosure further provides compositions comprising the compound of formula (I) or a pharmaceutically acceptable salt thereof for use in the treatment of cancer.

In one embodiment, the disease or disorder is anemia of chronic disease, anemia of chronic inflammation, anemia associated with cancer, or fibrodysplasia ossificans progressive.

In one embodiment, the disease or disorder is anemia.

In one embodiment, the anemia related condition is fatigue associated with cancer.

In one embodiment, the disease or disorder is MDS.

In one embodiment, the disease or disorder is anemia associated with MDS.

In one embodiment, the disease or disorder is anemia associated with very low, low or intermediate MDS. In one embodiment, the disease or disorder is anemia associated with low or intermediate MDS.

In one embodiment, the disease or disorder is transfusion dependent anemia associated with MDS. In one embodiment, the MDS is primary MDS. In one embodiment, the MDS is secondary MDS. In one embodiment, the MDS is very low-risk MDS, low-risk MDS or intermediate-risk MDS.

In one embodiment, the subject has MDS with single lineage dysplasia refractory anemia.

In one embodiment, the subject has MDS with ring sideroblasts and is intolerant, resistant or refractory to luspatercept.

In one embodiment, the cancer is a brain stem glioma, breast cancer, lung cancer, colon cancer, kidney cancer, ovarian cancer, prostate cancer, pancreatic cancer, head and neck cancer, hepatocellular cancer or carcinoma of the endometrium.

In one embodiment, the cancer is a myeloproliferative disorder, hematological cancer, or a solid tumor.

In one embodiment, the hematological cancer is lymphoma.

In one embodiment, the solid tumor is colorectal cancer, breast tumor, ovarian tumor, prostate tumor, pancreatic tumor, head and neck tumor, renal cell carcinoma, or hepatocellular carcinoma. In another embodiment, the solid tumor is colorectal cancer. In another embodiment, the solid tumor is metastatic colorectal cancer.

In some embodiments, the present disclosure further provides compositions comprising the compound of formula (I) or a pharmaceutically acceptable salt thereof for use in the treatment for fibrodysplasia ossificans progressive (FOP), endometrial cancer, and diffuse intrinsic pontine glioma (DIPG).

In one embodiment, the disease or disorder includes endometrial cancer and diffuse intrinsic pontine glioma (DIPG). In another embodiment, the disease or disorder is DIPG.

In one embodiment, the treatment includes selecting a treatment regimen for a subject based on the subject's genetic profile. Such genetic profiles may be produced in any suitable manner (e.g., microarrays, reverse transcription polymerase chain reaction (RT-PCR), RNA/DNA sequencing, etc.).

In some embodiments, the genetic profile comprises one or more mutations in an ACVR1 gene. In one embodiment, the treatment includes detecting one or more mutations in an ACVR1 gene. In one embodiment, the subject has a predetermined genetic profile comprising such mutation(s). In some embodiments, the one or more mutations in the ACVR1 gene comprise a missense mutation, a frameshift mutation, a duplication (i.e., copy number variation), a splice site mutation, or a combination thereof.

In one embodiment, the one or more mutations comprise (P197F198)L, C509S, D185G, D185N, D433N, E38FS, F265S, G225D, G264S, G328E, G328R, G328V, G328W, G356D, G50C, H320Y, I323V, K31E, K345Q, L196P, L251S, M34I, N100D, N481I, P115S, P455A, Q207E, Q278P, R201I, R206C, R206H, R258G, R258S, R307Q, R325A, R375C, R375P, R401M, R490H, S130F, S226N, S41F, S440G, S469C, S56L, T298S, V234M, V91M, W98R, or a combination thereof. In one embodiment, the one or more mutations in the ACVR1 gene comprise R206H, G328V, R258G, or a combination thereof. In certain embodiments, the one or more mutations in the ACVR1 gene comprise R206H.

In some embodiments, the one or more mutations in the ACVR1 gene comprise a missense mutation. In some embodiments, the missense mutation is C509S, D185N, D433N, F265S, G225D, H320Y, I323V, K31E, K345Q, M34I, N100D, N481I, P115S, P455A, Q278P, R206C, R401M, S130F, S226N, S41F, S41F, S440G, S469C, S56L, T298S, V234M, V91M, or W98R. In some embodiments, the one or more mutations in the ACVR1 gene comprise a frameshift mutation. In some embodiments, the frameshift mutation is E38fs. In some embodiments, the one or more mutations in the ACVR1 gene comprise a splice site mutation. In some embodiments, the splice site mutation is G264S.

In one embodiment, the treatment comprises:
(a) detecting the presence of one or more mutations in an ACVR1 gene of a subject;
(b) administering a composition comprising the compound of formula (I) or a pharmaceutically acceptable salt thereof to the subject according to the mutation status.

In one embodiment, the treatment comprises measuring a biomarker level.

In one embodiment, the biomarker is selected from hemoglobin, myoblast, platelet, neutrophil, hepcidin, red blood cell, hepcidin in serum and bone marrow aspirate; iron metabolism markers in serum selected from iron, ferritin, transferrin, soluble transferrin receptor [STR], and total iron binding capacity [TIBC]; cytokines in serum or plasma selected from CRP, EPO, IL-6, and TGF-beta 1; indicators of inhibition of signal transduction pathways in bone marrow aspirates selected from phosphorylation of SMAD-1, 2, 3, 5 and 8 in PBMCs, and bone marrow biopsy/aspirate mononuclear pellet.

In one embodiment, the biomarker is selected from cytokines in serum or plasma selected from CRP, EPO, IL-6, and TGF-beta 1; and indicators of inhibition of signal transduction pathways in bone marrow aspirates selected from phosphorylation of SMAD-1, 2, 3, 5 and 8 in PBMCs, and bone marrow biopsy/aspirate mononuclear pellet.

In one embodiment, the treatment comprises measuring the level of hemoglobin, myoblast, platelet, neutrophil, hepcidin, and/or red blood cell.

In one embodiment, the treatment comprises measuring the level of hepcidin.

In one embodiment, the treatment comprises:
(a) measuring a biomarker level;
(b) administering the composition comprising the compound of formula (I) or a pharmaceutically acceptable salt thereof according to the biomarker level.

In one embodiment, the treatment comprises improving one or more hematologic parameters in the subject, said improvement is selected from decreasing myoblasts, increasing hemoglobin, increasing platelets, increasing neutrophils, decreasing hepcidin, reducing units of red blood cell transfused, reducing frequency of transfusion, and reducing transfusion dependence.

In one embodiment, increasing hemoglobin is defined as increasing hemoglobin i) to 10 g/dL or more; or ii) by 1.5 g/dL or more compared to an amount measured prior to administration of the compound of formula (I) or the pharmaceutically acceptable salt. In one embodiment, the increase in hemoglobin is maintained for 8 weeks or 12 weeks in the absence of red blood cell transfusions.

In one embodiment, the subject is transfusion dependent and units of red blood cells transfused is reduced by 4 or more units compared to the units of red blood cells transfused for the same period of time prior to administration of the compound of formula (I) or the pharmaceutically acceptable salt. In one embodiment, the period of time is 8 weeks or 12 weeks.

In one embodiment, increasing platelets is defined as increasing the platelet count i) by 30×109/L or more; or ii) to 75×109/L or more. In one embodiment, the increase in platelets is maintained for 8 weeks or 12 weeks in the absence of red blood cell transfusions.

In one embodiment, increasing neutrophils is defined as increasing the neutrophil count i) by 0.5×109/L or more or ii) to 1.0×109/L or more. In one embodiment, the increase in neutrophil count is maintained for 8 weeks or 12 weeks in the absence of red blood cell transfusions.

In one embodiment, decreasing myoblasts is defined as decreasing myoblasts i) to be 5% or fewer of bone marrow cells; or ii) by 50% or more compared to a baseline amount measured prior to administration of the compound of formula (I) or the pharmaceutically acceptable salt. In one embodiment, the decrease in myoblasts is maintained for 8 weeks or 12 weeks.

In one embodiment, decreasing hepcidin is defined as decreasing hepcidin by 25% or more compared to a baseline amount measured prior to administration of the compound of formula (I) or the pharmaceutically acceptable salt.

### Dosage and Treatment Regimen

In any of the foregoing embodiments, the active agent or a pharmaceutically acceptable salt thereof, is administered in an effective amount, which will vary depending upon a variety of factors including the activity of the specific active agent employed; the metabolic stability and length of action of the active agent; the age, body weight, general health, sex, and diet of the patient; the mode and time of administration; the rate of excretion; the drug combination; the severity of the particular disorder or condition; and the subject undergoing therapy.

Toxicity and therapeutic efficacy of methods described herein can be determined by standard pharmaceutical procedures in cell cultures or experimental animals, e.g., by determining the IC₅₀ and the LD₅₀ for an administered active agent. For administration, effective amounts (also referred to as doses) can be initially estimated based on results from in vitro assays and/or animal model studies. For example, a dose can be formulated in animal models to achieve a circulating concentration range that includes an IC₅₀ as determined in cell culture against a particular target. The dosage may vary depending upon the dosage form employed and the route of administration utilized. The exact formulation, route of administration and dosage can be chosen by the individual physician in view of the patient's condition. (See, e.g., Goodman & Gilman's The Pharmacological Basis Of Therapeutics, Ch. 3, 9^{th} ed., Ed. by Hardman, J., and Limbard, L., McGraw-Hill, New York City, 1996, p. 46.)

Compositions that will be administered to a subject take the form of one or more dosage units, where for example, a tablet may be a single dosage unit, and a container of one or more therapeutic agents of the disclosure in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The pharmaceutical composition to be administered using certain embodiments of the methods of the disclosure will, in any event, contain an effective amount of the active agent, or a pharmaceutically acceptable salt thereof, for treatment of a disease in accordance with the teachings of embodiments of this disclosure.

The active agent described herein is effective over a wide dosage range. For example, in the treatment of adult humans, dosages from about 5 mg to about 500 mg, from about 10 mg to about 320 mg, from about 30 mg to about 240 mg per day, and from about 30 mg to about 180 mg per day are examples of dosages that are used in some embodiments. In some embodiments, the dose is about 30 mg to about 120 mg per day. In some embodiments, the dose is about 20 mg to about 30 mg per day. In some embodiments, the dose is about 20 mg to about 40 mg per day. In some embodiments, the dose is about 30 mg to about 40 mg per day. In some embodiments, the dose is about 30 mg to about 60 mg per day. In some embodiments, the dose is about 40 mg to about 50 mg per day. In some embodiments, the dose is about 60 mg to about 240 mg per day. In some embodiments, the dose is about 60 mg to about 180 mg per day. In some embodiments, the dose is about 60 mg to about 120 mg per day. In some embodiments, the dose is about 60 mg to about 90 mg per day. In some embodiments, the dose is about 90 mg to about 120 mg per day. In some embodiments, the dose is about 120 mg to about 240 mg per day. In some embodiments, the dose is about 120 mg to about 160 mg per day. In some embodiments, the dose is about 120 mg to about 180 mg per day. In some embodiments, the dose is about 180 mg to about 240 mg per day. In some embodiments, the dose is about 210 mg to about 240 mg per day. In some embodiments, the dose is about 210 mg to about 270 mg per day. In some embodiments, the dose is about 270 mg to about 325 mg per day.

In some embodiments, the dose is about 5 mg per day. In some embodiments, the dose is about 10 mg per day. In some embodiments, the dose is about 20 mg per day. In some embodiments, the dose is about 25 mg per day. In other embodiments, the dose is about 30 mg per day. In some embodiments, the dose is about 40 mg per day. In other embodiments, the dose is about 50 mg per day. In other embodiments, the dose is about 60 mg per day. In some embodiments, the dose is about 80 mg per day. In some embodiments, the dose is about 90 mg per day. In some embodiments, the dose is about 100 mg per day. In other embodiments, the dose is about 120 mg per day. In some embodiments, the dose is about 125 mg per day. In some embodiments, the dose is about 145 mg per day. In some embodiments, the dose is about 150 mg per day. In some embodiments, the dose is about 160 mg per day. In other embodiments, the dose is about 180 mg per day. In some embodiments, the dose is about 210 mg per day. In other embodiments, the dose is about 240 mg per day. In other embodiments, the dose is about 250 mg per day. In some embodiments, the dose is about 270 mg per day. In other embodiments, the dose is about 300 mg per day. In other embodiments, the dose is about 320 mg per day. In other embodiments, the dose is about 325 mg per day.

In some embodiments, the initial dose starts at 20 mg or 30 mg per day every day. Dose escalation proceeds with provisional dose level up to 40 mg, 60 mg, 90 mg, 120 mg, 160 mg, 210 mg, 270 mg, or further. Further respective dose increments of up to 25% from the first dose to the next may occur.

As another example, in the treatment of adult humans, dosages from about 5 mg to about 500 mg, from about 10 mg to about 320 mg, from about 30 mg to about 240 mg per week, and from about 30 mg to about 180 mg per week are examples of dosages that are used in some embodiments. In some embodiments, the dose is about 30 mg to about 120 mg per week. In some embodiments, the dose is about 30 mg to about 60 mg per week. In some embodiments, the dose is about 60 mg to about 240 mg per week. In some embodiments, the dose is about 60 mg to about 180 mg per week. In some embodiments, the dose is about 60 mg to about 120 mg per week. In some embodiments, the dose is about 120 mg to about 240 mg per week. In some embodiments, the dose is about 120 mg to about 180 mg per week. In some embodiments, the dose is about 180 mg to about 240 mg per week.

In some embodiments, the dose is about 5 mg per week. In some embodiments, the dose is about 10 mg per week. In some embodiments, the dose is about 25 mg per week. In other embodiments, the dose is about 30 mg per week. In other embodiments, the dose is about 60 mg per week. In other embodiments, the dose is about 90 mg per week. In other embodiments, the dose is about 120 mg per week. In some embodiments, the dose is about 125 mg per week. In other embodiments, the dose is about 180 mg per week. In other embodiments, the dose is about 240 mg per week. In other embodiments, the dose is about 250 mg per week. In other embodiments, the dose is about 270 mg per week. In other embodiments, the dose is about 320 mg per week. In other embodiments, the dose is about 325 mg per week.

In all such embodiments, a pediatric dose may be between about 80% to 100% of an adult dose.

In some embodiments, a dose is escalated. In one embodiment, the dose begins at 30 mg per week and escalates in 30 mg increments up to 120 mg per week. In one embodiment, the dose begins at 30 mg per week and remains level. In one embodiment, the dose begins at 30 mg per week and escalates to a final dose of 60 mg per week. In one embodiment, the dose begins at 30 mg per week and escalates to an interim dose of 60 mg per week, which further escalates to a final dose of 90 mg per week. In one embodiment, the dose begins at 30 mg per week and escalates to a first interim dose of 60 mg per week, a second interim dose of 90 mg per week, which further escalates to a final dose of 120 mg per week. In one embodiment, the dose begins at 60 mg per week and remains level. In one embodiment, the dose begins at 60 mg per week and escalates to a final dose of 90 mg per week. In one embodiment, the dose begins at 60 mg per week and escalates to an interim dose of 90 mg per week, which further escalates to a final dose of 120 mg per week.

In embodiments, an active agent is administered in a dose ranging from about 10 mg/m² to about 500 mg/m² per day. In embodiments, an active agent is administered in a dose ranging from about 150 mg/m² to about 350 mg/m² per day. In some embodiments, an active agent is administered in a dose ranging from about 200 mg/m² to about 300 mg/m² per day. In some embodiments, an active agent is administered in a dose ranging from about 220 mg/m² to about 260 mg/m² per day. In some embodiments, an active agent is administered in a dose ranging from about 230 mg/m² to about 250 mg/m² per day. In some embodiments, an active agent is administered in a dose ranging from about 235 mg/m² to about 245 mg/m² per day. In specific embodiments, an active agent is administered in a dose is about 240 mg/m² per day.

In embodiments, an active agent is administered in a dose ranging from about 10 mg/m² to about 500 mg/m² per week. In embodiments, an active agent is administered in a dose ranging from about 150 mg/m² to about 350 mg/m² per week. In some embodiments, an active agent is administered in a dose ranging from about 200 mg/m² to about 300 mg/m² per week. In some embodiments, an active agent is administered in a dose ranging from about 220 mg/m² to about 260 mg/m² per week. In some embodiments, an active agent is administered in a dose ranging from about 230 mg/m² to about 250 mg/m² per week. In some embodiments, an active agent is administered in a dose ranging from about 235 mg/m² to about 245 mg/m² per week. In specific embodiments, an active agent is administered in a dose is about 240 mg/m² per week.

The exact dosage will depend upon the active agent, the route of administration, the form in which the compound is administered, the subject to be treated, physical and physiological factors including target, body weight, severity of condition, type of cancer, previous or concurrent therapeutic interventions, idiopathy of the subject, and the preference and experience of the attending physician.

In some embodiments, an effective amount of an active agent is administered in a single dose. Typically, such administration will be by injection, e.g., intravenous injection, in order to introduce the agent quickly. However, other routes are used as appropriate. A single dose of a compound of the disclosure may also be used for treatment of an acute condition.

In some embodiments, an effective amount of an active agent is administered in multiple doses. In some embodiments, dosing is about once, twice, three times, four times, five times, six times, or more than six times per day. In some embodiments, dosing is about once, twice, three times, four times, five times, six times, or more than six times per week. In other embodiments, dosing is about once a month, once every two weeks, once a week, or once every other day. In yet another embodiment, the administration continues for more than about 6, 10, 14, 28 days, two months, six months, or one year. In some cases, continuous dosing is achieved and maintained as long as necessary. In some embodiments, an active agent is administered for 1, 7, 14, 21, or 28 consecutive days. In some embodiments, an active agent is administered weekly. In some embodiments, an active agent is administered on week 1, week 2, week 3, and week 4 of a four-week cycle. In some embodiments, an active agent is administered for at least 1, 2, 3, 4, 5, 6, 7, 8, 9, or 10 cycles. In some embodiments, an active agent is administered for at least 2 cycles. In some embodiments, an active agent is administered for at least 4 cycles. In some embodiments, an active agent is administered for at least 9 cycles. In some embodiments, a four-week cycle includes one or more holiday. In some embodiments, a four-week cycle does not include a holiday and dosing is continuous.

In various embodiments, the active agent is administered daily. In various embodiments, the active agent is administered weekly. In each of such embodiments, the active agent is taken substantially at the same time of day. In some embodiments, the active agent is administered after fasting (e.g., for at least six hours). In some embodiments, a subject fasts for at least one hour after administration.

Administration of an active agent may continue as long as necessary. In some embodiments, an active agent is administered for more than 1, 2, 3, 4, 5, 6, 7, 14, or 28 days. In some embodiments, an active agent is administered for less than 28, 14, 7, 6, 5, 4, 3, 2, or 1 day. In some embodiments, an active agent is administered for more than 1, 4, 8, 12, 16, 20, 24, 28, 32, 36, 40, 44, 48, or 52 weeks. In some embodiments, an active agent is administered for less than 52, 48, 44, 40, 36, 32, 28, 24, 20, 16, 12, 8, 4, or 1 week.

In some embodiments, an active agent is administered chronically on an ongoing basis, e.g., for the treatment of chronic effects.

In some embodiments, the additional therapeutic agent may be administered chronically (e.g., as a maintenance therapy). In other such embodiments, the additional one or more therapeutic agents may be administered as a second treatment regimen.

In some embodiments, an active agent is administered in dosages. Due to intersubject variability in compound pharmacokinetics, individualization of dosing regimen is provided in certain embodiments. Dosing for a therapeutic agent may be found by routine experimentation in light of the instant disclosure and/or can be derived by one of ordinary skill in the art.

Dosage amount and interval may be adjusted individually to provide plasma levels of the active species which are sufficient to maintain desired pharmacological effects. These plasma levels are referred to as minimal effective concentrations (MECs). Dosages necessary to achieve the MEC will depend on individual characteristics and route of administration. HPLC assays or bioassays can be used to determine plasma concentrations.

Dosage intervals may also be determined using MEC value. In some embodiments, methods of treatment comprise maintaining plasma levels above the MEC for 10-90% of the time. In some embodiments, plasma levels are maintained above the MEC between 30-90% of the time. In some embodiments, plasma levels are maintained above the MEC between 50-90% of the time. For example, in certain embodiments, effective amounts of a therapeutic agent may range from approximately 2.5 mg/m² to 1500 mg/m² per day. For example, in certain embodiments, effective amounts of a therapeutic agent may range from approximately 2.5 mg/m² to 1500 mg/m² per week. Additional illustrative amounts range from 0.2-1000 mg, 2-500 mg, and 20-250 mg either daily or weekly.

In cases of local administration or selective uptake, the effective local concentration of the therapeutic agent may not be related to plasma concentration, and other procedures known in the art may be employed to determine the correct dosage amount and interval.

### Combination Therapy

The compound of formula (I) or the pharmaceutically acceptable salt used in embodiments of the disclosure, or pharmaceutically acceptable derivatives thereof, may also be administered simultaneously with, prior to, or after administration of one or more other therapeutic agents. For example, a first therapeutic agent, including, but not limited to, an ACVR1 inhibitor, a JAK2 inhibitor, or a ALK5 inhibitor, can be administered and after a sufficient period of time a second therapeutic agent is administered. In such embodiments, the period of time between the administration of the first therapeutic agent and the second therapeutic agent may be referred to as a “treatment break” or “holiday.” A “treatment break” or “holiday” may also refer to a period of time between cycles of treatment. In some embodiments, such a treatment break or holiday ranges from about 12 hours to about 48 hours. In some embodiments, such a treatment break or holiday ranges from about 18 to about 36 hours. In some embodiments, such a treatment break or holiday ranges from about 24 to about 48 hours. In some embodiments, a treatment break or holiday ranges from about 2 to about 10 days. In some embodiments, a treatment break or holiday ranges from about 3 to about 5 days. In some embodiments, a treatment break or holiday ranges from about 5 to about 9 days. In some embodiments, a treatment break or holiday is about 7 days. In various embodiments, an active agent is administered for 21 consecutive days followed by a 7 day treatment break or holiday. In some embodiments, a treatment break or holiday is about 30 days. In various embodiments, an active agent is administered weekly for a cycle of 4 consecutive weeks without a treatment break or holiday between cycles. One of ordinary skill in the art can derive an appropriate dosing schedule based on common techniques and knowledge. In embodiments, an active agent and one or more of radiation therapy and an additional therapeutic agent are administered sequentially.

The therapeutic agents may be used in the combination therapy includes, but not limited to, chemotherapeutic agents, anti-cancer agents, MTAP inhibitors, EGFR antibodies, MET inhibitors, Platelet-derived Growth Factor (PDGF) receptor inhibitors, Phosphoinositide 3-kinase (PI3K) inhibitors, Cyclin-Dependent Kinase (CDK) inhibitors, Cyclin-Dependent Kinase (CDK) inhibitors, p53-MDM2 inhibitors, Mitogen-activated protein kinase (MEK) inhibitors, B-RAF inhibitors, ALK inhibitors or immune checkpoint inhibitors.

In some embodiments, the chemotherapeutic agents are selected from mitotic inhibitors, alkylating agents, anti-metabolites, cell cycle inhibitors, enzymes, topoisomerase inhibitors, biological response modifiers, anti-hormones, antiangiogenic agents, anti-androgens, platinum coordination complexes, methylhydrazine derivatives, adrenocortical suppressants, aminoglutethimide, hormone and hormone antagonists progestins estrogens, antiestrogens, androgens, and aromatase inhibitors.

In some embodiments, the chemotherapeutic agents are selected from pemetrexed (Alimta®), gemcitabine (Gemzar®), 5-fluorouracil (Adrucil®, Carac® and Efudex®), methotrexate (Trexall®), capecitabine (Xeloda®), floxuridine (FUDR®), decitabine (Dacogen®), azacitidine (Vidaza® and Azadine®), 6-mercaptopurine (Purinethol®), cladribine (Leustatin®, Litak® and Movectro®), fludarabine (Fludara®), pentostatin (Nipent®), nelarabine (Arranon®), clofarabine (Clolar® and Evoltra®), and cytarabine (Cytosar®).

In one embodiment, the chemotherapeutic agent is selected from the group consisting of mitotic inhibitors, alkylating agents, anti-metabolites, cell cycle inhibitors, enzymes, topoisomerase inhibitors such as CAMPTOSAR (irinotecan), biological response modifiers, anti-hormones, antiangiogenic agents such as MMP-2, MMP-9 and COX-2 inhibitors, anti-androgens, poly(ADP-ribose) polymerase (PARP) inhibitors, tyrosine kinase inhibitors (imatinib mesylate, dasatinib, nilotinib, bosutinib, etc.), platinum coordination complexes (cisplatin, etc.), taxanes (Taxol, Taxotere, etc.), substituted ureas such as hydroxyurea; methylhydrazine derivatives, e.g., procarbazine; adrenocortical suppressants, e.g., mitotane, aminoglutethimide, hormone and hormone antagonists such as the adrenocorticosteriods (e.g., prednisone), progestins (e.g., hydroxyprogesterone caproate), estrogens (e.g., diethylstilbesterol), antiestrogens such as tamoxifen, androgens, e.g., testosterone propionate, and aromatase inhibitors, such as anastrozole, and AROMASIN (exemestane).

Examples of alkylating agents that the above method can be carried out in combination with include, without limitation, fluorouracil (5-FU) alone or in further combination with leukovorin; other pyrimidine analogs such as UFT, capecitabine, gemcitabine and cytarabine, the alkyl sulfonates, e.g., busulfan (used in the treatment of chronic granulocytic leukemia), improsulfan and piposulfan; aziridines, e.g., benzodepa, carboquone, meturedepa and uredepa; ethyleneimines and methylmelamines, e.g., altretamine, triethylenemelamine, triethylenephosphoramide, triethylenethiophosphoramide and trimethylolmelamine; and the nitrogen mustards, e.g., chlorambucil (used in the treatment of chronic lymphocytic leukemia, primary macroglobulinemia and non-Hodgkin's lymphoma), cyclophosphamide (used in the treatment of Hodgkin's disease, multiple myeloma, neuroblastoma, breast cancer, ovarian cancer, lung cancer, Wilm's tumor and rhabdomyosarcoma), estramustine, ifosfamide, novembrichin, prednimustine and uracil mustard (used in the treatment of primary thrombocytosis, non-Hodgkin's lymphoma, Hodgkin's disease and ovarian cancer); and triazines, e.g., dacarbazine (used in the treatment of soft tissue sarcoma).

Examples of immune checkpoint inhibitors that the above method can be carried out in combination with include, without limitation, PD-1 inhibitors, such as pembrolizumab (also known as Lambrolizumab, MK-3475, MK03475, SCH-900475, or KEYTRUDA®) and other anti-PD-1 antibodies (as disclosed in Hamid, O. et al. (2013) New England Journal of Medicine 369 (2): 134-44, U.S. Pat. No. 8,354,509, and WO 2009/114335, incorporated by reference in their entirety), nivolumab (also known as MDX-1106, MDX-1106-04, ONO-4538, BMS-936558, or OPDIVO®) and other anti-PD-1 antibodies (as disclosed in U.S. Pat. No. 8,008,449 and WO 2006/121168, incorporated by reference in their entirety), cemiplimab (LIBTAYO®), spartalizumab (PDR001), pidilizumab (CureTech), MEDI0680 (Medimmune), cemiplimab (REGN2810), dostarlimab (TSR-042), PF-06801591 (Pfizer), sinitilimab, toripalimab, tislelizumab (BGB-A317), camrelizumab (INCSHR1210, SHR-1210), AMP-224 (Amplimmune), CBT-501 (CBT Pharmaceuticals), CBT-502 (CBT Pharmaceuticals), JS001 (Junshi Biosciences), IBI308 (Innovent Biologics), INCSHR1210 (Incyte), also known as SHR-1210 (Hengrui Medicine), BGBA317 (Beigene), BGB-108 (Beigene), BAT-I306 (Bio-Thera Solutions), GLS-010 (Gloria Pharmaceuticals; WuXi Biologics), AK103, AK104, AK105 (Akesio Biopharma; Hangzhou Hansi Biologics; Hanzhong Biologics), LZM009 (Livzon), HLX-10 (Henlius Biotech), MEDI0680 (Medimmune), PDF001 (Novartis), PF-06801591 (Pfizer), Pidilizumab (CureTech) also known as CT-011 and other anti-PD-1 antibodies (as disclosed in Rosenblatt, J. et al. (2011) J Immunotherapy 34(5): 409-18, U.S. Pat. Nos. 7,695,715, 7,332,582, and 8,686,119, incorporated by reference in their entirety), REGN2810 (Regeneron), TSR-042 (Tesaro) also known as ANB011, or CS1003 (CStone Pharmaceuticals) MEDI0680 (Medimmune), is also known as AMP-514 MEDI0680 and other anti-PD-1 antibodies are disclosed in U.S. Pat. No. 9,205,148 and WO 2012/145493, incorporated by reference in their entirety. Further known anti-PD-1 antibody molecules include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety. In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety. In one embodiment, the anti-PD-1 antibody molecule comprises the CDRs, variable regions, heavy chains and/or light chains of BAP049-Clone-E or BAP049-Clone-B disclosed in US 2015/0210769. The antibody molecules described herein can be made by vectors, host cells, and methods described in US 2015/0210769, incorporated by reference in its entirety. In one embodiment, the PD-1 inhibitor is a peptide that inhibits the PD-1 signaling pathway, e.g., as described in U.S. Pat. No. 8,907,053, incorporated by reference in its entirety. In one embodiment, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In one embodiment, the PD-1 inhibitor is AMP-224 (B7-DCIg (Amplimmune), e.g., disclosed in WO 2010/027827 and WO 2011/066342, incorporated by reference in their entirety).

Examples of immune checkpoint inhibitors that the above method can be carried out in combination with include, without limitation, PD-L1 inhibitors, such as atezolizumab (also known as MPDL3280A, RG7446, RO5541267, YW243.55.570, or TECENTRIQ®) and other anti-PD-L1 antibodies as disclosed in U.S. Pat. No. 8,217,149, incorporated by reference in its entirety, avelumab (BAVENCIO® also known as MSB0010718C) and other anti-PD-L1 antibodies as disclosed in WO 2013/079174, incorporated by reference in its entirety, durvalumab (IMFINZI® or MEDI4736) and other anti-PD-L1 antibodies as disclosed in U.S. Pat. No. 8,779,108, incorporated by reference in its entirety), FAZ053 (Novartis), and BMS-936559 (Bristol-Myers Squibb). In certain embodiments, the PD-L1 inhibitor is KN035 (Alphamab; 3DMed; Ascletis Pharma), Envafolimab (TRACON Pharmaceuticals), BMS 936559 (Bristol-Myers Squibb), CS1001 (CStone Pharmaceuticals, Ligand Pharmaceuticals), CX-072 (CytomX Therapeutics), FAZ053 (Novartis), SHR-1316 (Hengrui Medicine), TQB2450 (Chiatai Tianqing), STI-A1014 (Zhaoke Pharm; Lee's Pharm, Lonza, Sorrento Therapeutics, NantWorks), LYN00102 (Lynkcell), A167 (Harbour BioMed, Kelun Group), BGB-A333 (Beigene), MSB2311 (Mabspace Biosciences), or HLX-20 (Henlius Biotech). In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entirety. In certain embodiments, the PD-L1 inhibitor is Cosibelimab (Fortress Biotech), LY3300054 or Iodapolimab (Eli Lilly), GS-4224 (Gilead Sciences), STI-A1015 (Yuhan, Sorrento Therapeutics), BCD-135 (BIOCAD), Cosibelimab (Dana-Farber Cancer Institute, TG Therapeutics), APL-502 (Apollomics), AK106 (Akeso Biopharma), MSB2311 (Transcenta Holding), TG-1501 (TG Therapeutics), FAZ053 (Novartis). In certain embodiments, the PD-L1 inhibitor is MT-6035 (Molecular Templates), Icaritin and ZKAB001 (Lonza, Lee's Pharmaceutical Holdings, Sorrento Therapeutics, Shenogen Pharma Group), TRIDENT Antibody (MacroGenics, Zai Lab), YBL-007 (Anh-Gook Pharmaceutical, Y-Biologics), HTI-1316 (Hengrui Therapeutics), PD-L1 Oncology Project (Weizmann Institute of Sciences), JS003 (Shanghai Junshi Biosciences), ND021 (Numab Therapeutics, CStone Pharmaceuticals), Toca 521 (Tocagen), STT01 (STCube). In certain embodiments, the PD-L1 inhibitor is DB004 (DotBio), MT-5050 (Molecular Templates), KD036 (Kadmon). In one embodiment, the PD-L1 inhibitor is an anti-PD-L1 antibody molecule. In one embodiment, the PD-L1 inhibitor is an anti-PD-L1 antibody molecule as disclosed in US 2016/0108123, published on Apr. 21, 2016, entitled “Antibody Molecules to PD-L1 and Uses Thereof,” incorporated by reference in its entirety. In one embodiment, the anti-PD-L1 antibody molecule comprises the CDRs, variable regions, heavy chains and/or light chains of BAP058-Clone 0 or BAP058-Clone N disclosed in US 2016/0108123.

Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entirety.

### EXAMPLES

### Example 1—Characterizing the Active Pharmaceutical Ingredient

The hydrochloride salt of the compound of formula (I) was formulated into three (3) oral dose strengths (5, 25, and 125 mg dose [based on free base]). The physicochemical properties of the compound of formula (I), the active pharmaceutical ingredient (API), are summarized in Table 1.

TABLE 1

| Chemical name | N2-[3-methoxy-4-(4-methylpiperazin-1yl)phenyl]-N4- |
|---|---|
| | [2-(pyridin-2-yl)pyridin 3-yl] pyrimidine-2,4-diamine |
| | hydrochloride |
| Molecular | C₂₆H₂₉CIN₈O |
| formula | |
| Molecular | 505.05 (468.2386076) |
| Weight | |
| (g/mole) | |
| Structure | 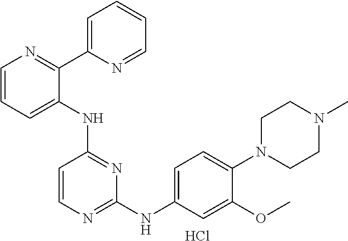 [SMILES: COc1cc(Nc2nccc(Nc3cccnc3-c3ccccn3)n2)ccc1N1CCN(C)CC1 ; Cl] |
| cLog P | 3.98 |
| Log D (pH 7.4) | 2.66 |

The molecular weight provided in parentheses is that of the free base.

The solubility of the API was tested. In all media, solubility of the compound is good, namely the maximum tested dose of 125 mg was found soluble in 250 mL of media. As such, the compound was characterized as highly soluble for this dose, with reference to BCS classification. This characterization may not be accurate for larger doses. Reference is made to Table 2.

TABLE 2

| | Solubility | | Maximum dose mg |
|---|---|---|---|
| Media | (mg/mL) | pH | solubility in 250 ml |
| 0.1N HCl | 1.027 | 1.005 | 256 |
| pH 4.5 Acetate buffer | 1.015 | 4.508 | 253 |
| pH 6.8 Phosphate | 0.980 | 6.812 | 245 |
| buffer | | | |
| Water | 1.008 | 6.213 | 252 |

An initial excipient compatibility study was performed. The results are shown in Table 3A and 3B.

TABLE 3A
Compatible excipients, one or more of which may be used in an
embodiment of the present disclosure.

| Name | Full name | Function |
|---|---|---|
| MCCPH102 | Microcrystalline cellulose PH102 | Diluent/Filler |
| Lactose | N/A | Diluent/Filler |
| DCP | Dicetylphosphate | Emulsifier |
| SLS | Sodium Lauryl Sulfonate | Lubricant |
| Poloxamer P188 | Poly(ethylene glycol)-block- | Surfactant |
| | poly(propylene glycol)-block- | |
| | poly(ethylene glycol) | |
| Aerosil 200 | Hydrophilic fumed silica | Lubricant? |
| Ac-di-sol | Croscarmellose sodium | Disintegrant |
| Kollidone CL | Crospovidone CL | Disintegrant |
| Kollidone 30 | Crospovidone 30 | Disintegrant |
| Klucel EXF | Hydroxypropylcellulose | Diluent/Filler |
| Magnesium stearate | N/A | Lubricant |
| SSF | Sodium Stearyl Fumarate | Lubricant |
| Mannitol | N/A | Diluent/Filler |
| Starch 1500 | N/A | Diluent/Filler |

TABLE 3B
Excipient compatibility of compound of formula (I) (“API”) capsules at
40° C./75% RH-open glass vial-4 week
Excipient Compatibility Study

| | | Unknown RRT | | | | | | | | | | |
|---|---|---|---|---|---|---|---|---|---|---|---|---|
| | | 0.162 | 0.866 | 0.989 | 1.046 | 1.07 | 1.08 | 1.341 | 1.38 | 1.549 | 1.633 | Total |
| Initial | API | 0.12 | ND | BQL | BQL | 0.07 | 0.06 | 0.27 | 0.08 | 0.21 | 0.12 | 0.93 |
| 40°/ | API | 0.11 | BQL | BQL | BQL | 0.07 | 0.05 | 0.2 | 0.1 | 0.19 | 0.12 | 0.84 |
| 75%- | API + | 0.08 | 0.11 | BQL | 0.05 | 0.07 | 0.05 | ND | 0.09 | 0.17 | 0.1 | 0.72 |
| open- | MCCPH | | | | | | | | | | | |
| 4 | 102 | | | | | | | | | | | |
| week | Lactose | 0.09 | BQL | BQL | BQL | 0.06 | BQL | 0.12 | 0.08 | 0.16 | 0.1 | 0.61 |
| | DCP | 0.09 | BQL | BQL | 0.06 | 0.07 | BQL | 0.13 | 0.08 | 0.16 | ND | 0.59 |
| | SLS | 0.1 | ND | BQL | BQL | BQL | BQL | 0.05 | ND | 0.07 | ND | 0.22 |
| | Poloxamer | 0.09 | BQL | BQL | ND | 0.06 | BQL | 0.16 | 0.08 | 0.16 | 0.1 | 0.65 |
| | P188 | | | | | | | | | | | |
| | Aerosil 200 | 0.12 | 0.06 | 0.05 | 0.05 | 0.08 | 0.06 | 0.13 | 0.1 | 0.2 | 0.12 | 0.97 |
| | Ac-di-sol | 0.09 | 0.05 | BQL | BQL | 0.06 | BQL | 0.12 | 0.09 | 0.17 | 0.1 | 0.68 |
| | Kollidone | 0.1 | 0.06 | BQL | BQL | 0.06 | 0.35 | 0.09 | 0.08 | 0.18 | 0.11 | 1.03 |
| | CL | | | | | | | | | | | |
| | Kollidone | 0.09 | 0.05 | BQL | BQL | 0.06 | 0.18 | 0.11 | 0.08 | 0.17 | 0.1 | 0.84 |
| | 30 | | | | | | | | | | | |
| | Klucel EXF | 0.09 | 0.07 | BQL | BQL | 0.06 | 0.06 | 0.05 | 0.07 | 0.16 | 0.09 | 0.65 |
| | Magnesium | 0.11 | 0.06 | BQL | BQL | 0.07 | 0.05 | 0.11 | 0.09 | 0.2 | 0.12 | 0.81 |
| | stearate | | | | | | | | | | | |
| | SSF | 0.11 | 0.06 | BQL | BQL | 0.07 | 0.05 | 0.13 | 0.09 | 0.2 | 0.12 | 0.83 |
| | Mannitol | 0.09 | 0.07 | BQL | BQL | 0.06 | BQL | 0.05 | 0.08 | 0.15 | 0.09 | 0.59 |
| | Starch | 0.09 | 0.12 | BQL | BQL | 0.07 | BQL | ND | 0.09 | 0.19 | 0.11 | 0.67 |
| | 1500 | | | | | | | | | | | |
| | Empty | 0.13 | BQL | BQL | BQL | 0.08 | 0.05 | 0.21 | 0.11 | 0.22 | 0.14 | 0.94 |
| | HCG | | | | | | | | | | | |
| | capsule | | | | | | | | | | | |
| | Blend 1- | 0.09 | 0.07 | BQL | BQL | 0.07 | BQL | 0.06 | 0.08 | 0.17 | 0.1 | 0.64 |
| | 10 mg | | | | | | | | | | | |
| | Blend 2- | 0.1 | 0.07 | BQL | BQL | 0.06 | BQL | 0.06 | 0.08 | 0.16 | 0.1 | 0.63 |
| | 20 mg | | | | | | | | | | | |
| | Blend-3 | 0.11 | BQL | BQL | BQL | 0.08 | 0.05 | 0.19 | 0.1 | 0.19 | 0.12 | 0.84 |
| | 240 mg | | | | | | | | | | | |

In the compatibility study, all the samples were evaluated for two (2) primary properties: appearance and related substances. Except Kollidone CL and Kollidone 30 (PVP-K30) all the excipients were found to be compatible with API and showed no sign of change in appearance (color and texture) and % level of related substance during the compatibility study.

As demonstrated by Table 3B, all three prototype formulations were found stable at 40° C./75% relative humidity (RH; open) for 4 weeks (W) and at 50° C. (close) for 4W with respect to their related substance profile.

Regarding formulation development, a direct blending strategy with manual capsule filing was selected. The compound of formula (I), as an API, was characterized to have medium flow properties. In other words, direct blending is a smooth and robust process for formulation development of a dosage form. Initially, a direct blending was selected as a first strategy. Capsules were filled by manual capsule filling machine.

### Example 2—Oral Solid Formulation

Increasing amounts of active pharmaceutical ingredient were formulated into three similar blends, see, Table 4. The product was formulated for immediate release using common excipients in the blend. The drug was placed in #3, hard gelatin capsules.

TABLE 4
Excipients in the Blend, 5, 25, and 125 mg Strength Capsules

| Excipient | Purpose |
|---|---|
| Microcrystalline Cellulose | Diluent/Filler |
| Lactose Monohydrate | Diluent/Filler |
| Croscarmellose Sodium | Disintegrant |
| Magnesium Stearate | Lubricant |

### Example 3—Prototype Compositions

### Example 3A

A prototype batch of compound of formula (I) as API capsule of 5 mg was planned to evaluate the physical and chemical property of a finished dosage form.

The test capsule was filled by a manual capsule filling machine.

TABLE 5
Prototype 5 mg Strength

| | Batch Size | | |
|---|---|---|---|
| | 700 capsules | | |
| | Batch No | | |
| | 63AG(499)056 | | |
| | mg/cap | % w/w per batch | g/batch |
| Comp of formula (I) HCl as | 5.400 | 3.180 | 3.780 |
| API | | | |
| Microcrystalline Cellulose | 36.80 | 21.65 | 25.76 |
| (Avicel PH-102) | | | |
| Lactose Monohydrate spray | 121.0 | 71.18 | 84.70 |
| dried fast flow | | | |
| Croscarmellose Sodium | 5.100 | 3.000 | 3.570 |
| (Ac-di-sol) | | | |
| Magnesium stearate | 1.700 | 1.000 | 1.190 |
| Total Weight of blend | 170.0 | 100.0 | 119.0 |
| Empty Capsule Average | 46.00 | NA | NA |
| weight | | | |
| Total filed Capsule weight | 216.0 | | |

Note:
Assay, water content and Residual substance was not taken for API calculation

The following trituration and blending process was performed:
Sift the API and Excipient through 40 # screen.
Place the batch quantity of microcrystalline cellulose and croscarmellose sodium in the mixing vessel of the Turbula blender and run the blender for 1 minute at 42 rpm. Using the pestle coat the mortar with a portion (approximately 3 gram) lactose monohydrate.
Weigh & add the quantity of lactose monohydrate, equivalent to twice the weight of the API into the mortar. Add API to the mortar. Blend API and lactose monohydrate approximately 2 minutes using the pestle.
Weigh & add the quantity of lactose monohydrate, equivalent to the weight of API & lactose Monohydrate blend (above step) to the mortar. Blend for approximately 2 minutes using the pestle.
Weigh & add the quantity of lactose monohydrate equivalent to the weight of API & lactose monohydrate blend (above step) to the mortar. Blend for approximately 2 minutes using the pestle.
Transfer the triturate from mortar to Turbula blender containing microcrystalline cellulose and croscarmellose sodium.
Rinse the mortar and pestle with remaining lactose monohydrate and transfer to the blender.
Mix the API and excipient in Turbula blender and run the blender at 42 rpm for 15 minutes.
Sift magnesium stearate through 40# and add sifted magnesium stearate in Turbula blender and blend at 42 rpm for an additional 1 minutes.

For the manual capsule filling, the following process was performed:
The accurate quantity of lubricated blend was weighted for the quantity of 300 capsules and record in following step;
Theoretical weight of the lubricated blend was taken: 51.0 g;
Weight of the lubricated blend was taken with 1.5% overages was taken; 51.765 g; and
Normal tapping was given to fill the capsule by manual capsule filling machine.

Dissolution in pH 1.2 was then tested.

TABLE 6
Dissolution data in pH 1.2

| | Dissolution Condition | |
|---|---|---|
| | 0.1N HCl (pH 1.2), 50 rpm, paddle with sinker, 900 ml | |
| Time Point | % Drug Released | % RSD |
| 0 | 0 | 0 |
| 5 | 89 | 5.9 |
| 10 | 93 | 4.8 |
| 15 | 94 | 4.5 |
| 20 | 94 | 4.4 |
| 30 | 94 | 4.0 |
| 45 | 95 | 3.9 |
| Recovery | 95 | 3.5 |

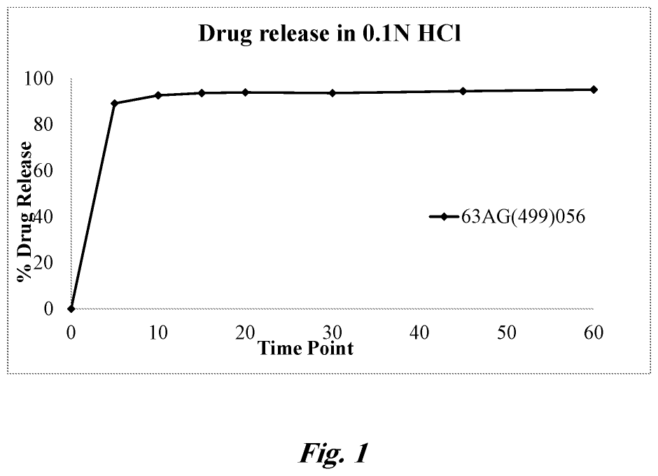

As further illustrated in FIG. 1, the physical and chemical parameters of the prototype capsule were found satisfactory and within targeted limits. More than 85% drug release was observed within 15 minutes. Content uniformity and assay values were within specification limits. The assay value was on lower side. Samples were loaded on stability on accelerated condition (40° C./75% RH up to 6 month) and long term condition (25° C./60% RH up to 6 month) to evaluate the related substance, assay, dissolution and water content.

### Example 3B

A prototype batch of compound of formula (I) as API capsule of 25 mg was planned to evaluate the physical and chemical property of a finished dosage form.

Capsules were filled by a manual capsule filling machine.

TABLE 7
Prototype 25 mg Strength

| | Batch Size | | |
|---|---|---|---|
| | 700 capsules | | |
| | Batch No | | |
| | 63AH(499)063 | | |
| | mg/cap | % w/w per batch | g/batch |
| Comp of formula (I) | 27.00 | 15.88 | 18.90 |
| HCl | | | |
| Microcrystalline | 31.50 | 18.53 | 22.05 |
| Cellulose (Avicel PH- | | | |
| 102) | | | |
| Lactose Monohydrate | 104.7 | 61.59 | 73.29 |
| spray dried fast flow | | | |
| Croscarmellose Sodium | 5.100 | 3.000 | 3.570 |
| (Ac-di-sol) | | | |
| Magnesium stearate | 1.700 | 1.000 | 1.190 |
| Total Weight of blend | 170.0 | 100.0 | 119.0 |
| Empty Capsule | 46.00 | NA | NA |
| Average weight | | | |
| Total filed Capsule | 216.0 | | |
| weight | | | |

The following trituration and blending process was performed:
Sift the API and excipient through 40 # screen.
Place the batch quantity of microcrystalline cellulose and croscarmellose sodium in the mixing vessel of the Turbula blender and run the blender for 1 minute at 42 rpm. Using the pestle coat the mortar with a portion (approximately 3 gram) lactose monohydrate.
Place lactose monohydrate (approximately 20 gram) into the mortar.
Add API to the mortar.
Blend the API and lactose monohydrate for approximately 2 minutes using the pestle.
Add lactose monohydrate (approximately 20 gram) to mortar. Blend for approximately 2 minutes using the pestle.
Add lactose monohydrate (approximately 20 gram) to mortar. Blend for approximately 2 minutes using the pestle.
Transfer the triturate from mortar to Turbula blender containing microcrystalline cellulose and croscarmellose sodium.
Rinse the mortar and pestle with remaining lactose monohydrate and transfer to the blender, Rinse to the mixing vessel containing microcrystalline cellulose and croscarmellose sodium.
Mix the API and excipient in Turbula blender and run the blender at 42 rpm for 15 minutes.
Sift magnesium stearate through 40# and add sifted magnesium stearate in Turbula blender and blend at 42 rpm for an additional 1 minutes.

For the manual capsule filling, the following process was performed:
The accurate quantity of lubricated blend was weighted for the quantity of 300 capsules and record in following step.
Theoretical weight of the lubricated blend was taken: 51.0 g.
Weight of the lubricated blend was taken with 1.5% overages was taken; 51.765 g
Normal tapping was given to fill the capsule by manual capsule filling machine.

Dissolution in pH 1.2 was then tested.

TABLE 8
Dissolution data in pH 1.2

| | Dissolution Condition | |
|---|---|---|
| | 0.1N HCl (pH 1.2), 50 rpm, paddle with sinker, 900 ml | |
| Time Point | % Drug Released | % RSD |
| 0 | 0 | 0 |
| 5 | 89 | 5.1 |
| 10 | 93 | 2.2 |
| 15 | 95 | 1.8 |
| 20 | 96 | 2.2 |
| 30 | 96 | 2.6 |
| 45 | 97 | 3.4 |
| Recovery | 97 | 3.9 |

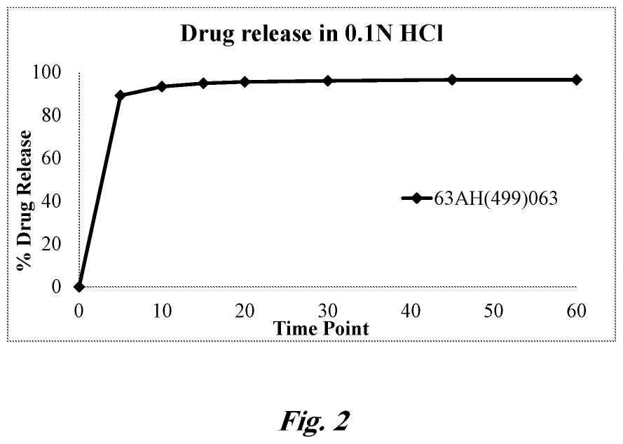

As further illustrated in FIG. 2, the physical and chemical parameters of the 25 mg prototype capsule was found satisfactory and within targeted limits. More than 85% drug release was observed within 15 minutes. Content uniformity and assay values were within specification limit. Samples were loaded on stability on accelerated conditions (40° C./75% RH up to 6 months) and long term condition (25° C./60% RH up to 6 months) to evaluate the related substance, assay, dissolution, and water content.

### Example 3C

A prototype batch of compound of formula (I) as API capsule of 125 mg was planned to evaluate the physical and chemical property of finished dosage form. Capsules were filled by manual capsule filling machine.

TABLE 9
Prototype 125 mg Strength

| | Batch Size | | |
|---|---|---|---|
| | 700 capsules | | |
| | Batch No | | |
| | 63AI(499)070 | | |
| | mg/cap | % w/w per batch | g/batch |
| Comp of formula (I) | 135.0 | 35.3 | 94.50 |
| HCl as API | | | |
| Microcrystalline | 53.00 | 13.95 | 37.10 |
| Cellulose (Avicel PH- | | | |
| 102) | | | |
| Lactose Monohydrate | 176.7 | 46.5 | 123.69 |
| spray dried fast flow | | | |
| Croscarmellose Sodium | 11.50 | 3.0 | 8.05 |
| (Ac-di-sol) | | | |
| Magnesium stearate | 3.800 | 1.0 | 2.66 |
| Total Weight of blend | 380.0 | 100.0 | 266.0 |
| Empty Capsule | 93.00 | NA | NA |
| Average weight | | | |
| Total filed Capsule | 473.0 | | |
| weight | | | |

The following trituration and blending process was performed:
Sift the API and excipient through 40 # screen.
Sift the microcrystalline cellulose and croscarmellose sodium through 40# and add into Turbula blender (Capacity: 2 L) and run the blender for 1 minute at 42 rpm.
Cosift the API with lactose monohydrate through 40# and add into turbula blender having microcrystalline cellulose and croscarmellose sodium.
Mix the API and excipient in Turbula blender and run the blender at 42 rpm for 15 minutes.
Sift magnesium stearate through 40# and add sifted magnesium stearate in Turbula blender and blend at 42 rpm for an additional 1 minutes.

For the manual capsule filling, the following process was performed:
The accurate quantity of lubricated blend was weighted for the quantity of 300 capsules and record in following step.
Theoretical weight of the lubricated blend was taken: 114.0 g
Weight of the lubricated blend was taken with 1.5% overages was taken; 115.8 g
Normal tapping was given to fill the capsule by manual capsule filling machine.

Dissolution in pH 1.2 was then tested.

TABLE 10
Dissolution data in pH 1.2

| | Dissolution Condition | |
|---|---|---|
| | 0.1N HCl (pH 1.2), 50 rpm, paddle with sinker, 900 ml | |
| Time Point | % Drug Released | % RSD |
| 0 | 0 | 0 |
| 5 | 79 | 10.8 |
| 10 | 88 | 7.3 |
| 15 | 92 | 5.6 |
| 20 | 93 | 5.2 |
| 30 | 95 | 4.2 |
| 45 | 97 | 3.6 |
| Recovery | 98 | 3.0 |

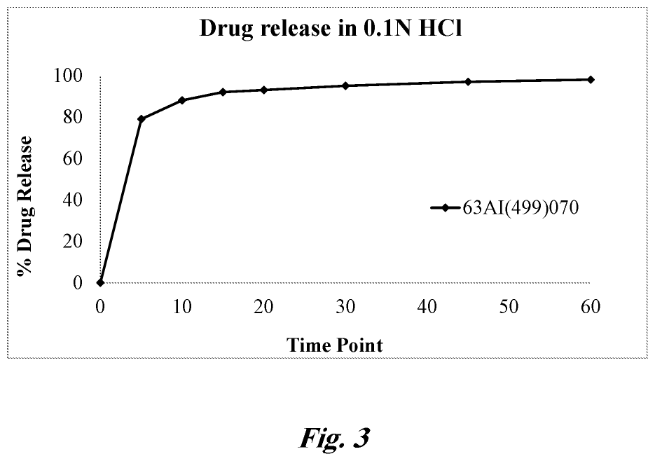

As further illustrated in FIG. 3, the physical and chemical parameters of the capsules were found satisfactory and within targeted limits. More than 85% drug release was observed within 15 minutes. Assay values were within specification limit. Samples were loaded on stability on accelerated condition (40° C./75% RH up to 6 months) and long term condition (25° C./60% RH up to 6 months) to evaluate the related substance, assay, dissolution and water content.

### Example 4—Finished Form Compositions

As noted hereinabove, for the 5 mg strength batch, the drug content was observed to be on the lower side. So, a repeat batch was made to evaluate the drug content in a finished dosage form.

Prototype batch of API capsule 5 mg was evaluated for physical and chemical properties of finished dosage form. Batch size was same as above, and capsules filled by manual capsule filling machine.

TABLE 11
Formula Composition of API Capsule 5 mg

| | Batch Size | | |
|---|---|---|---|
| | 700 capsules | | |
| | Batch No | | |
| | 63AG(499)083 | | |
| | mg/cap | % w/w per batch | g/batch |
| Comp of formula (I) HCl as | 5.400 | 3.180 | 3.780 |
| API | | | |
| Microcrystalline Cellulose | 36.80 | 21.65 | 25.76 |
| (Avicel PH-102) | | | |
| Lactose Monohydrate spray | 121.0 | 71.18 | 84.70 |
| dried fast flow | | | |
| Croscarmellose Sodium | 5.100 | 3.000 | 3.570 |
| (Ac-di-sol) | | | |
| Magnesium stearate | 1.700 | 1.000 | 1.190 |
| Total Weight of blend | 170.0 | 100.0 | 119.0 |
| Empty Capsule Average | 46.00 | NA | NA |
| weight | | | |
| Total filed Capsule weight | 216.0 | | |

Note:
Assay, water content and Residual substance was not taken for API calculation

The following blending process was performed:
Sift the API and Excipient through 40 # screen.
Sift microcrystalline cellulose and croscarmellose sodium through 40# and place in the mixing vessel of the Turbula blender and run the blender for 1 minute at 42 rpm.
Cosift lactose monohydrate with API equivalent to twice time of API quantity through 40# and add in Turbula blender mix it for 5 minute at 42 rpm.
Sift the twice quantity of lactose monohydrate (above step) and add into Turbula blender mix it for 5 minute at 42 rpm.
Sift the twice quantity of lactose monohydrate (above step) and add into Turbula blender mix it for 5 minute at 42 rpm.
Sift remaining quantity of lactose monohydrate and add into Turbula blender mix it for 10 minute at 42 rpm.
Sift magnesium stearate through 40 # and add sifted magnesium stearate in Turbula blender and blend at 42 rpm for an additional 3 minutes.

The following manual capsule filling process was performed:
The accurate quantity of lubricated blend was weighted for the quantity of 300 capsules and record in following step
Theoretical weight of the lubricated blend was taken: 51.0 g
Weight of the lubricated blend was taken with 1.5% overages was taken; 51.765 g
Normal tapping was given to fill the capsule by manual capsule filling machine.

The physical and chemical parameters of the capsules were found satisfactory and within targeted limits. Content uniformity of the finish dosage form was within specification limits. There was no significant drug variation found. The average value of content uniformity was slightly on the lower side. So, for the next batch, the assay of API on dried basis, water content and residual solvent was incorporated for API calculation and planned batch to evaluate the content uniformity.

As noted hereinabove, a prototype batch of API capsule 5 mg (2000 capsule batch size) was planned to evaluate the physical and chemical property of finished dosage form. Batch size was higher (to check the API loss during manufacturing in lower batch size) as compare to prior batches and capsule filled by manual capsule filling machine. In this batch assay, water content and residual solvent were calculated for API calculation.

TABLE 12
Formula Composition of API Capsule 5 mg

| | Batch Size | | |
|---|---|---|---|
| | 2000 capsules | | |
| | Batch No | | |
| | 63AG(536)006 | | |
| | mg/cap | % w/w per batch | g/batch |
| Comp of formula (I) | 5.400 | 3.180 | 10.80 |
| HCl as API | | | |
| Microcrystalline | 36.80 | 21.65 | 73.60 |
| Cellulose (Avicel PH- | | | |
| 102) | | | |
| Lactose Monohydrate | 121.0 | 71.18 | 242.0 |
| spray dried fast flow | | | |
| Croscarmellose Sodium | 5.100 | 3.000 | 10.20 |
| (Ac-di-sol) | | | |
| Magnesium stearate | 1.700 | 1.000 | 3.400 |
| Total Weight of blend | 170.0 | 100.0 | 340.0 |
| Empty Capsule | 46.00 | NA | NA |
| Average weight | | | |
| Total filed Capsule | 216.0 | | |
| weight | | | |

Note:
Assay, water content and Residual solvent was incorporated for API calculation

The following blending process was performed:
Sift microcrystalline cellulose and croscarmellose sodium through 40# and place in the mixing vessel of the Turbula blender and run the blender for 1 minute at 42 rpm.
Cosift lactose monohydrate with API equivalent to twice time of API quantity through 40# and add in Turbula blender mix it for 5 minute at 42 rpm.
Sift the twice quantity of lactose monohydrate (above step) and add into Turbula blender mix it for 5 minute at 42 rpm.
Sift the twice quantity of lactose monohydrate (above step) and add into Turbula blender mix it for 5 minute at 42 rpm.
Sift remaining quantity of lactose monohydrate and add into Turbula blender mix it for 15 minute at 42 rpm.
Sift magnesium stearate through 40# and add sifted magnesium stearate in Turbula blender and blend at 42 rpm for an additional 3 minutes.

The following manual capsule filing process was performed:
The accurate quantity of lubricated blend was weighted for the quantity of 300 capsules and record in following step
Theoretical weight of the lubricated blend was taken: 51.0 g
Weight of the lubricated blend was taken with 1.5% overages was taken; 51.765 g
Normal tapping was given to fill the capsule by manual capsule filling machine.

The physical and chemical parameters of capsules were found satisfactory and within targeted limits. Content uniformity of the finish dosage form was within specification limits. There was no significant drug variation found. After increasing batch size, overall assay of the drug content was increased. So, in next batch of API Capsules 5 mg, an increase in the batch size was made to evaluate the content uniformity.

### Example 5—Confirmatory Compositions

Direct blending is a preferred process for formulation development. So, direct blending strategy was selected as a first strategy. Initially, capsules were filled by manual capsule filling machines. Manual filling is time consuming and less productive compared to automatic capsule filling machines. Therefore, confirmatory batches were filled by both automatic capsule filling machine and manual capsule filling machine to evaluate impact on physical and chemical property of finished dosage form across all strengths.

### Example 5A: 5 mg Strength

A test formula was finalized for API capsule 5 mg. A confirmatory batch was planned with a higher batch size. Quantitative formula and manufacturing process were held consistent from prior batches (above). Capsules were filled by both automatic capsule filing machine and manual capsule filling machine. Confirmatory batch and clinical batch of API capsule 5 mg was planned with GMP API.

TABLE 13
Formula Composition of API Capsule 5 mg

| | Batch Size | | |
|---|---|---|---|
| | 7000 capsules | | |
| | Batch No | | |
| | 63AG(536)022 | | |
| | mg/cap | % w/w per batch | g/batch |
| Comp of formula (I) | 5.400 | 3.180 | 37.80 |
| HCl as API | | | |
| Microcrystalline | 36.80 | 21.65 | 257.6 |
| Cellulose (Avicel PH- | | | |
| 102) | | | |
| Lactose Monohydrate | 121.0 | 71.18 | 847.0 |
| spray dried fast flow | | | |
| Croscarmellose Sodium | 5.100 | 3.000 | 35.70 |
| (Ac-di-sol) | | | |
| Magnesium stearate | 1.700 | 1.000 | 11.90 |
| Total Weight of blend | 170.0 | 100.0 | 1190 |
| Empty Capsule | 50.00 | NA | NA |
| Average weight | | | |
| Total filed Capsule | 220.0 | | |
| weight | | | |

Note:
Assay, water content and Residual solvent was incorporated for API calculation

The following blending process was performed:
Sift microcrystalline cellulose and croscarmellose sodium through 40# and place in the mixing vessel of the Turbula blender and run the blender for 1 minute at 42 rpm.
Cosift lactose monohydrate with API equivalent to twice time of API quantity through 40# and add in Turbula blender mix it for 5 minute at 42 rpm.
Sift the twice quantity of lactose monohydrate (above step) and add into Turbula blender mix it for 5 minute at 42 rpm.
Sift the twice quantity of lactose monohydrate (above step) and add into Turbula blender mix it for 5 minute at 42 rpm.
Sift remaining quantity of lactose monohydrate and add into Turbula blender mix it for 15 minute at 42 rpm.
Sift magnesium stearate through 40# and add sifted magnesium stearate in Turbula blender and blend at 42 rpm for an additional 3 minutes.

The following capsule filling process was performed:
Capsules were filled by using both an automatic capsule filling machine and manual capsule filling machine. Lubricated blend was divided into 2 parts. Approximate 970 gram of blend for automatic capsule filling and remaining lubricated blend was separated for manual capsule filling machine.

For the automatic capsule filling, the following parameters were used:
Dosing disc Thickness: 14 mm
Dosing disc Diameter: 4.6 mm

TABLE 14
Automatic Capsule Filling Machine Setting Parameters

| | | At 1000 | At 2000 | At 3000 | At 4000 | |
|---|---|---|---|---|---|---|
| Trial | At Initial | Capsule | Capsule | Capsule | Capsule | At End |
| Machine | 80 | 80 | 80 | 80 | 80 | 80 |
| speed (rpm) | | | | | | |
| Tamping | Pin 1: 25 | Pin 1: 25 | Pin 1: 25 | Pin 1: 25 | Pin 1: 25 | Pin 1: 25 |
| pin settings | Pin 2: 25 | Pin 2: 25 | Pin 2: 25 | Pin 2: 25 | Pin 2: 25 | Pin 2: 25 |
| (mm) | Pin 3: 25 | Pin 3: 25 | Pin 3: 25 | Pin 3: 25 | Pin 3: 25 | Pin 3: 25 |
| | Pin 4: 12 | Pin 4: 12 | Pin 4: 12 | Pin 4: 12 | Pin 4: 12 | Pin 4: 12 |
| | Pin 5: 14 | Pin 5: 14 | Pin 5: 14 | Pin 5: 14 | Pin 5: 14 | Pin 5: 14 |

For the manual capsule filing, the following process was performed:
The accurate quantity of lubricated blend was weighted for the quantity of 300 capsules and record in following step
Theoretical weight of the lubricated blend was taken: 51.0 g
Weight of the lubricated blend was taken with 1.5% overages was taken; 51.765 g
Normal tapping was given to fill the capsule by manual capsule filling machine.

In Process Quality Control (IPQC) tests were performed.

TABLE 15
IPQC parameters of the filled capsule by automatic capsule filling

| | IPQC of capsules | | | | | |
|---|---|---|---|---|---|---|
| | | At 1000 | At 2000 | At 3000 | At 4000 | |
| Parameters | Initial | capsule | capsule | capsule | capsule | At End |
| Appearance | Swedish Orange capsule size ‘3’ | | | | | |
| Avg. Weight | 220.37 | 222.76 | 220.91 | 222.29 | 221.08 | 215.28 |
| (mg) of 10 | | | | | | |
| capsules | | | | | | |
| Individual | 218.7- | 220.6- | 217.5- | 221.0- | 218.7- | 213.1- |
| Weight | 221.8 | 225.1 | 223.5 | 224.2 | 224.1 | 217.8 |
| Variation | | | | | | |
| (mg) | | | | | | |
| RSD % | 0.51 | 0.63 | 0.84 | 0.44 | 0.87 | 0.68 |

| | |
|---|---|
| Locking | 15.6-15.8 |
| length (mm) | |
| Disintegration | 1 min 18 sec-1 min 32 sec |
| Time | |
| (minute: | |
| second) | |

Weight Sorting

| | |
|---|---|
| Total Sorted Capsule | 4500 |
| Accepted Capsule | 4500 |
| Rejected Capsule | 0 |

Table 16
IPQC parameters of filled capsule by manual capsule filling

| | IPQC of capsules | | | |
|---|---|---|---|---|
| Parameters | Plate 1 | Plate 2 | Plate 3 | Plate 4 |
| Appearance | Swedish Orange capsule size ‘3’ | | | |
| Avg. Weight | 220.46 | 218.23 | 217.46 | 218.38 |
| (mg) of | | | | |
| 10 capsules | | | | |
| Individual | 211.7-230.5 | 209.6-225.3 | 211.4-222.3 | 212.3-225.8 |
| Weight | | | | |
| Variation | | | | |
| (mg) | | | | |
| RSD % | 2.39 | 2.82 | 1.80 | 1.87 |

| | |
|---|---|
| Locking | 15.70-15.83 |
| length (mm) | |
| Disintegration | 1 min-1 min 22 sec |
| Time | |
| (minute: | |
| second) | |

Weight Sorting

| | |
|---|---|
| Total Sorted | 1198 |
| Capsule | |
| Accepted | 1189 |
| Capsule | |
| Rejected | 9 |
| Capsule | |

The automated capsule machine was run at 80 rpm. In 1 minute, 80 capsules were filled with high accuracy. As per the IPQC data hereinabove, the relative standard deviation (RSD) value was less than 1% and no capsules were rejected. With the manual capsule filing machine, the RSD value was less than 3%. Chemical analysis was performed on both sets of capsules.

TABLE 17
Content Uniformity

| | Batch No | |
|---|---|---|
| | 63AG(536)022 -A | 63AG(536)022- B |
| | Automatic capsule filling | Manual capsule filling |
| | machine | machine |
| Sr. No. | % Drug Content | % Drug Content |
| 1 | 96.50 | 94.40 |
| 2 | 98.60 | 90.00 |
| 3 | 96.30 | 86.80 |
| 4 | 98.00 | 92.00 |
| 5 | 92.80 | 87.20 |
| 6 | 98.50 | 93.00 |
| 7 | 95.00 | 92.60 |
| 8 | 99.40 | 77.20 |
| 9 | 95.80 | 91.70 |
| 10 | 96.10 | 90.10 |
| Avg | 96.70 | 89.50 |
| SD | 1.98 | 4.96 |
| RSD | 2.05 | 5.54 |
| Minimum | 92.80 | 77.20 |
| Maximum | 99.40 | 94.40 |
| AV Value | 6.50 | 20.90 |
| Average Assay | 95.2 | ND |

Dissolution in pH 1.2 was then tested.

TABLE 18
Dissolution data in pH 1.2

| | Dissolution Condition | |
|---|---|---|
| | 0.1N HCl (pH 1.2), 50 rpm, paddle with sinker, 900 ml | |
| Time Point | % Drug Released | % RSD |
| 0 | 0 | 0 |
| 5 | 88 | 4.1 |
| 10 | 96 | 1.6 |
| 15 | 97 | 1.4 |
| 20 | 97 | 1.2 |
| 30 | 98 | 1.4 |
| 45 | 98 | 1.7 |
| Recovery | 98 | 1.6 |

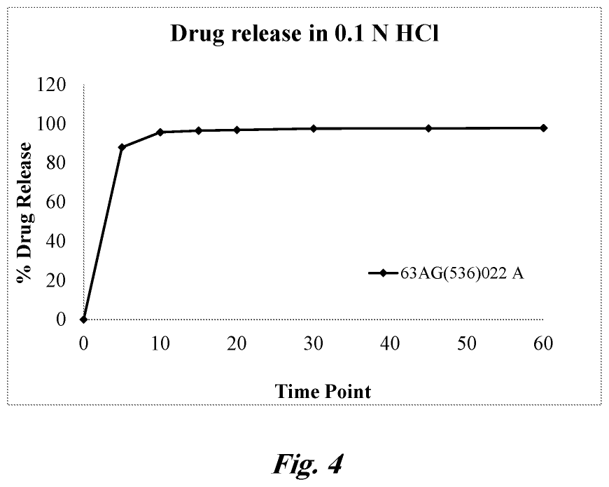

As illustrated in FIG. 4, capsules filled by the automatic capsule filling machine: physical and chemical parameters were found satisfactory and within targeted limits. More than 85% drug release was observed within 15 minutes in 0.1 N HCl. Content uniformity and assay values were within specification limits. Samples were loaded on stability on accelerated conditions (40° C./75% RH up to 6 months) and long term conditions (25° C./60% RH up to 6 months) to evaluate the related substance, assay, dissolution and water content.

Capsules filled by the manual capsule filling machine: physical parameters of capsules were found satisfactory. Content uniformity of capsules was out of the specification limit. Acceptance value was higher than 20. So, in final, clinical batch, capsules will be filled by automatic capsule filling machine.

### Example 5B: 25 mg Strength

A formulation of was finalized for API capsule 25 mg. A confirmatory batch was planned with higher batch size. Quantitative formula and manufacturing process was same as prior batches herein described. Capsules were filled by both automatic capsule filing machine and manual capsule filling machine. Confirmatory batch and clinical batch of API capsule 25 mg was planned with GMP API.

TABLE 19
Formula Composition of API Capsule 25 mg

| | Batch Size | | |
|---|---|---|---|
| | 7000 capsules | | |
| | Batch No | | |
| | 63AH(536)039 | | |
| | mg/cap | % w/w per batch | g/batch |
| Comp of Formula (1) | 27.00 | 15.88 | 189.0 |
| HCl as API | | | |
| Microcrystalline | 31.50 | 18.53 | 220.5 |
| Cellulose (Avicel PH- | | | |
| 102) | | | |
| Lactose Monohydrate | 104.7 | 61.59 | 732.9 |
| spray dried fast flow | | | |
| Croscarmellose Sodium | 5.100 | 3.000 | 35.70 |
| (Ac-di-sol) | | | |
| Magnesium stearate | 1.700 | 1.000 | 11.90 |
| Total Weight of blend | 170.0 | 100.0 | 1190 |
| Empty Capsule | 50.00 | NA | NA |
| Average weight | | | |
| Total filed Capsule | 220.0 | | |
| weight | | | |

Note:
Assay, water content and Residual solvent was incorporated for API calculation

The following blending process was performed:
Sift the API and Excipient through 40 # screen.
Sift microcrystalline cellulose and croscarmellose sodium through 40# and place in the mixing vessel of the Turbula blender and run the blender for 1 minute at 42 rpm.
Cosift lactose monohydrate with API equivalent to weight of API quantity through 40# and add in Turbula blender and mix it for 5 minute at 42 rpm.
Sift the same quantity of lactose monohydrate (above step) and add into Turbula blender mix it for 5 minute at 42 rpm.
Sift the same quantity of lactose monohydrate (above step) and add into Turbula blender mix it for 5 minute at 42 rpm.
Sift remaining quantity of lactose monohydrate and add into Turbula blender mix it for 15 minute at 42 rpm.
Sift magnesium stearate through 40# and add sifted magnesium stearate in Turbula blender and blend at 42 rpm for an additional 3 minutes.

Capsules were filled by using automatic capsule filling machine and manual capsule filling machine. Lubricated blend was divided into 2 parts. Approximate 1000 gram of blend for automatic capsule filling and remaining lubricated blend was separated for manual capsule filling machine.

For the automatic capsule filling of the portioned batch the following parameters were used:
Dosing disc Thickness: 14 mm
Dosing disc Diameter: 4.6 mm

TABLE 20
Automatic capsule filling machine setting parameters

| | | At 1000 | At 2000 | At 3000 | At 4000 | At 5000 | At |
|---|---|---|---|---|---|---|---|
| Trial | At Initial | Capsule | Capsule | Capsule | Capsule | Capsule | End |
| Machine | 80 | 80 | 80 | 80 | 80 | 80 | 80 |
| speed | | | | | | | |
| (rpm) | | | | | | | |
| Tamping | Pin 1: 25 | Pin 1: 25 | Pin 1: 25 | Pin 1: 25 | Pin 1: 25 | Pin 1: 25 | Pin 1: 25 |
| pin | Pin 2: 25 | Pin 2: 25 | Pin 2: 25 | Pin 2: 25 | Pin 2: 25 | Pin 2: 25 | Pin 2: 25 |
| settings | Pin 3: 25 | Pin 3: 25 | Pin 3: 25 | Pin 3: 25 | Pin 3: 25 | Pin 3: 25 | Pin 3: 25 |
| (mm) | Pin 4: 25 | Pin 4: 25 | Pin 4: 25 | Pin 4: 25 | Pin 4: 25 | Pin 4: 25 | Pin 4: 25 |
| | Pin 5: 14 | Pin 5: 14 | Pin 5: 14 | Pin 5: 14 | Pin 5: 14 | Pin 5: 14 | Pin 5: 14 |

The following process was used for the portion of manual capsule filling:
The accurate quantity of lubricated blend was weighted for the quantity of 300 capsules and record in following step
Theoretical weight of the lubricated blend was taken: 51.0 g
Weight of the lubricated blend was taken with 1.5% overages was taken; 51.765 g
Normal tapping was given to fill the capsule by manual capsule filling machine.

TABLE 21
IPQC Parameters of filled capsule by automatic capsule filling

| | IPQC of capsules | | | | | | |
|---|---|---|---|---|---|---|---|
| | | At | At | At | | | |
| | | 1000 | 2000 | 3000 | At 4000 | At 5000 | |
| Parameters | Initial | capsule | capsule | capsule | capsule | capsule | At End |
| Appearance | Dark Green capsule size ‘3’ | | | | | | |
| Avg. Weight | 222.23 | 220.95 | 220.35 | 221.34 | 221.07 | 219.72 | 215.67 |
| (mg) of 10 | | | | | | | |
| capsules | | | | | | | |
| Individual | 220.6- | 218.5- | 218.2- | 218.9- | 219.2- | 217.7- | 213.8- |
| Weight | 223.4 | 222.2 | 222.5 | 222.8 | 224.9 | 224.0 | 217.8 |
| Variation | | | | | | | |
| (mg) | | | | | | | |
| RSD % | 0.41 | 0.52 | 0.65 | 0.48 | 0.93 | 0.79 | 0.55 |

| | |
|---|---|
| Locking | 15.6-15.9 |
| length (mm) | |
| Disintegration | 1 min 15 sec-1 min 29 sec |
| Time | |
| (minute: | |
| second) | |

Weight Sorting

| | |
|---|---|
| Total Sorted Capsule | 5568 |
| Accepted Capsule | 5568 |
| Rejected Capsule | 0 |

TABLE 22
IPQC Parameters of filled capsule by manual capsule filling

| | IPQC of capsules | |
|---|---|---|
| Parameters | Plate 1 | Plate 2 |
| Appearance | Dark green capsule size ‘3’ | |
| Avg. Weight (mg) of 10 capsules | 221.11 | 221.62 |
| Individual Weight Variation (mg) | 213.3-224.8 | 218.4-225.2 |
| RSD % | 1.60 | 1.04 |
| Locking length (mm) | 15.70-15.85 | |
| Disintegration Time (minute: | 1 min 18-1 min 25 sec | |
| second) | | |

Weight Sorting

| | |
|---|---|
| Total Sorted Capsule | 558 |
| Accepted Capsule | 548 |
| Rejected Capsule | 10 |

The automated capsule machine was run at 80 rpm. In 1 minute, 80 capsules were filled with high accuracy. As per the IPQC data herein noted, the RSD value was less than 1% and no capsules were rejected. In the manual capsule filing machine portion, the RSD value was less than 2%. Chemical analysis was performed on both sets of capsules.

Content Uniformity

TABLE 23
Content uniformity and assay

| | Batch No | |
|---|---|---|
| | 63AH(536)039-A | 63AH(536)039-B |
| | Automatic capsule | Manual capsule |
| | filling machine | filling machine |
| Sr. No. | % Drug Content | % Drug Content |
| 1 | 95.40 | 94.00 |
| 2 | 96.00 | 107.00 |
| 3 | 97.90 | 97.40 |
| 4 | 97.50 | 95.40 |
| 5 | 107.40 | 100.60 |
| 6 | 95.90 | 94.90 |
| 7 | 111.40 | 103.70 |
| 8 | 97.40 | 95.60 |
| 9 | 93.70 | 98.40 |
| 10 | 97.10 | 103.80 |
| Avg | 98.97 | 99.08 |
| SD | 5.71 | 4.48 |
| RSD | 5.77 | 4.52 |
| Minimum | 93.70 | 94.00 |
| Maximum | 111.40 | 107.00 |
| AV Value | 13.70 | 10.80 |
| Average Assay | 99.1 | ND |

Dissolution in pH 1.2 was then tested.

TABLE 24
Dissolution data in pH 1.2

| | Dissolution Condition | |
|---|---|---|
| | 0.1 N HCl (pH 1.2), 50 rpm, | |
| | paddle with sinker, 900 ml | |
| Time Point | % Drug Released | % RSD |
| 0 | 0 | 0 |
| 5 | 88 | 7.3 |
| 10 | 96 | 2.9 |
| 15 | 96 | 2.6 |
| 20 | 97 | 2.5 |
| 30 | 97 | 2.7 |
| 45 | 97 | 2.7 |
| Recovery | 97 | 2.8 |

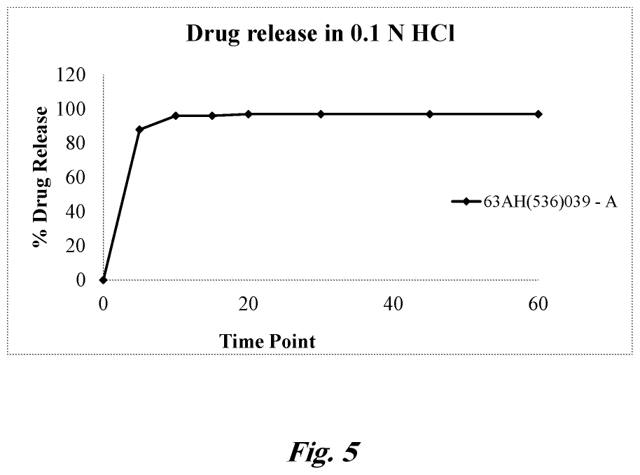

As illustrated in FIG. 5, capsules filled by the automatic capsule filling machine: the physical and chemical parameters of capsule was found satisfactory and within targeted limit. More than 85% drug release was observed within 15 minutes in 0.1N HCl. Content uniformity and assay value were within specification limit. Samples were loaded on stability on accelerated condition (40° C./75% RH up to 6 months) and long term condition (25° C./60% RH up to 6 months) to evaluate the related substance, assay, dissolution and water content.

Capsules filled by the manual capsule filling machine: physical and chemical parameters of capsules were found satisfactory. Content uniformity of capsules was within specification limits. Manual capsule filling, however, is very lengthy and less accurate when compared to the results demonstrated for automatic capsule filling machine. As per the physical characterization, capsules were filled by automatic capsule filling machine having less weight variation compare to manual capsule filling machine. So, for clinical batches, capsules will be filled by automatic capsule filling machine.

### Example 5C: 125 mg Strength

A formula of was finalized for API capsule 125 mg. A confirmatory batch was planned with higher batch size. Quantitative formula and manufacturing process was same as prior described batches. Capsules were filled by an automatic capsule filing machine. A confirmatory batch and clinical batch of API capsules at 125 mg was planned with GMP API.

TABLE 25
Formula Composition of API Capsules 125 mg

| | Batch Size | | |
|---|---|---|---|
| | 2800 capsules | | |
| | Batch No | | |
| | 63AI(536)031 | | |
| | mg/cap | % w/w per batch | g/batch |
| Comp of Formula (1) | 135.0 | 35.53 | 378.0 |
| HCl as API | | | |
| Microcrystalline | 53.10 | 13.97 | 148.7 |
| Cellulose (Avicel PH- | | | |
| 102) | | | |
| Lactose Monohydrate | 176.7 | 46.50 | 494.8 |
| spray dried fast flow | | | |
| Croscarmellose Sodium | 11.40 | 3.000 | 31.92 |
| (Ac-di-sol) | | | |
| Magnesium stearate | 3.800 | 1.000 | 10.64 |
| Total Weight of blend | 380.0 | 100.0 | 1064 |
| Empty Capsule | 90.00 | NA | NA |
| Average weight | | | |
| Total filed Capsule | 470.0 | | |
| weight | | | |

Note:
Assay, water content and Residual solvent was incorporated for API calculation

The following blending process was performed:
Sift microcrystalline cellulose and Croscarmellose Sodium through 40# and Place in the mixing vessel of the Turbula blender and run the blender for 1 minute at 42 rpm.
Cosift lactose monohydrate with API through 40# and add in turbula blender having Microcrystalline cellulose and Croscarmellose sodium.
Mix the API and excipient in turbula blender for 15 minute at 42 rpm.
Sift magnesium stearate through 40# and add sifted Magnesium Stearate in Turbula blender and blend at 42 rpm for an additional 3 minutes.

Capsules were filled by using automatic capsule filling machine. The following parameters were used:
Dosing disc Thickness: 16 mm
Dosing disc Diameter: 6.4 mm

TABLE 26
Automatic capsule filing machine setting parameters

| | Trial | | | | |
|---|---|---|---|---|---|
| | | At 1000 | At 1500 | At 2000 | At |
| | At Initial | Capsule | Capsule | Capsule | End |
| Machine | 80 | 80 | 80 | 80 | 80 |
| speed (rpm) | | | | | |
| Tamping pin | Pin 1: 25 | Pin 1: 25 | Pin 1: 25 | Pin 1: 25 | Pin 1: 25 |
| settings (mm) | Pin 2: 25 | Pin 2: 25 | Pin 2: 25 | Pin 2: 25 | Pin 2: 25 |
| | Pin 3: 25 | Pin 3: 25 | Pin 3: 25 | Pin 3: 25 | Pin 3: 25 |
| | Pin 4: 25 | Pin 4: 25 | Pin 4: 25 | Pin 4: 25 | Pin 4: 25 |
| | Pin 5: 15 | Pin 5: 15 | Pin 5: 15 | Pin 5: 15 | Pin 5: 14 |

TABLE 27
IPQC Parameters of filled capsule by automatic capsule filling

| | IPQC of capsules | | | | |
|---|---|---|---|---|---|
| | | At 1000 | At 1500 | At 2000 | |
| Parameters | Initial | capsule | capsule | capsule | At End |
| Appearance | Brown Opaque capsule size ‘0’ | | | | |
| Avg. Weight | 470.72 | 467.68 | 470.02 | 472.70 | 463.68 |
| (mg) of 10 | | | | | |
| capsules | | | | | |
| Individual | 465.7- | 462.5- | 459.8- | 470.4- | 455.7- |
| Weight | 474.9 | 471.8 | 477.6 | 474.1 | 479.0 |
| Variation (mg) | | | | | |
| RSD % | 0.58 | 0.69 | 0.99 | 0.28 | 1.50 |

| | |
|---|---|
| Locking length | 21.1-21.23 |
| (mm) | |
| Disintegration | 1 min 10 sec-1 min 27 sec |
| Time (minute: | |
| second) | |

Weight Sorting

| | |
|---|---|
| Total Sorted Capsule | 1975 |
| Accepted Capsule | 1957 |
| Rejected Capsule | 18 |

The automated capsule machine run at 80 rpm. In 1 minute 80 capsules were filled with high accuracy. Physical parameter of finish dosage form was within specification limit.

TABLE 28
Uniformity of dosage form by weight variation and assay

| | Batch No |
|---|---|
| | 63AI(536)031 |
| | Automatic capsule filling machine |
| Sr. No. | Uniformity of dosage form by weight variation |
| 1 | 101.10 |
| 2 | 100.70 |
| 3 | 101.70 |
| 4 | 99.60 |
| 5 | 98.80 |
| 6 | 100.10 |
| 7 | 100.00 |
| 8 | 102.60 |
| 9 | 101.50 |
| 10 | 99.80 |
| Avg | 100.59 |
| SD | 1.14 |
| RSD | 1.14 |
| Minimum | 98.80 |
| Maximum | 102.60 |
| AV Value | 2.8 |
| Average Assay | 100.5 |

Dissolution in pH 1.2 was then tested.

TABLE 29
Dissolution data in pH 1.2

| | Dissolution Condition | |
|---|---|---|
| | 0.1 N HCl (pH 1.2), 50 rpm, paddle with sinker, 900 ml | |
| Time Point | % Drug Released | % RSD |
| 0 | 0 | 0 |
| 5 | 80 | 14.9 |
| 10 | 90 | 5.9 |
| 15 | 95 | 3.2 |
| 20 | 96 | 1.9 |
| 30 | 98 | 1.3 |
| 45 | 99 | 1.8 |
| Recovery | 100 | 1.7 |

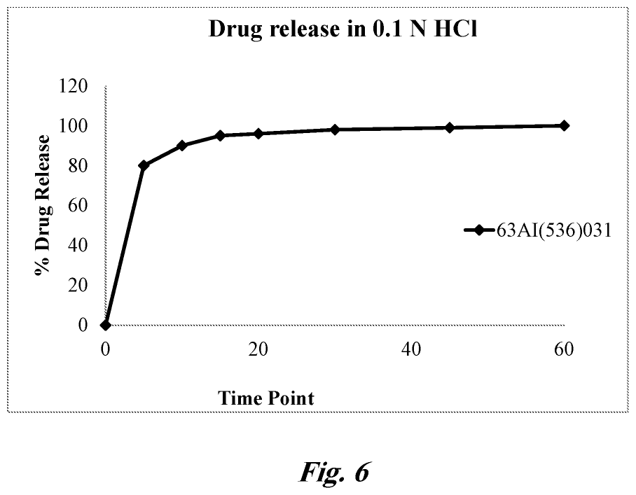

As illustrated in FIG. 6, capsules filled by an automatic capsule filling machine: physical and chemical parameters of capsule was found satisfactory and within targeted limits. More than 85% drug release was observed within 15 minutes in 0.1N HCl. Uniformity of dosage unit by weight variation and assay values were within specification limits. Samples were loaded on stability on accelerated condition (40° C./75% RH up to 6 months) and long term condition (25° C./60% RH up to 6 months) to evaluate the related substance, assay, dissolution and water content.

In clinical batches, the capsules will be filled by automatic capsule filling machine. The dissolution of confirmatory batch was found more than 85% in 15 minutes in pH 1.2. The content uniformity of this batch was found to be excellent, with AV Value less than 15. Other physical and chemical parameters were also found satisfactory.

As a result of testing, based on physical and chemical properties of capsules for all 3 strengths, clinical batches were planned. These batches will be loaded on stability to evaluate their physical and chemical parameters under different stability conditions.

### Example 6: Clinical Compositions

Based on the physiochemical properties of confirmatory batches of all three strengths, pilot GMP batches were performed and found satisfactory. Summarized information of GMP batches are given below.

TABLE 30
Composition of Clinical Batch API Capsule: 5 mg, 25 mg, 125 mg

| | | Standard | | Standard | | Standard | |
|---|---|---|---|---|---|---|---|
| Sr. | | Qty. | | Qty. | | Qty. | |
| No. | Ingredients | (mg)/Cap | % w/w | (mg)/Cap | % w/w | (mg)/Cap | % w/w |
| Comp of formula (I) Capsules | | 5 mg | | 25 mg | | 125 mg | |
| Batch No. | | 6363AG01 | | 6363AH01 | | 6363AI01 | |
| Batch Size | | 7000 Capsule | | 7000 Capsule | | 7000 Capsule | |
| 1. | Comp of formula | 5.400 | 3.18 | 27.00 | 15.88 | 135.0 | 35.53 |
| | (I) HCl (API) | | | | | | |
| | (# 40 sieve | | | | | | |
| | passed) | | | | | | |
| 2. | Microcrystalline | 36.80 | 21.65 | 31.50 | 18.53 | 53.10 | 13.97 |
| | Cellulose (Avicel | | | | | | |
| | PH102) | | | | | | |
| | (# 40 sieve passed) | | | | | | |
| 3. | Lactose, | 121.0 | 71.18 | 104.7 | 61.59 | 176.7 | 46.50 |
| | Monohydrate, | | | | | | |
| | Compendial | | | | | | |
| | [Modified Spray | | | | | | |
| | Dried, 316 Fast | | | | | | |
| | Flow] (# 40 sieve | | | | | | |
| | passed) | | | | | | |
| 4. | Croscarmellose | 5.100 | 3.000 | 5.100 | 3.000 | 11.40 | 3.000 |
| | Sodium (Ac-Di- | | | | | | |
| | Sol) | | | | | | |
| | (# 40 sieve | | | | | | |
| | passed) | | | | | | |
| 5. | Magnesium | 1.700 | 1.000 | 1.700 | 1.000 | 3.800 | 1.000 |
| | Stearate (Non- | | | | | | |
| | bovine) (# 40 | | | | | | |
| | sieve passed) | | | | | | |
| Total | | 170.0 | 100.0 | 170.0 | 100.00 | 380.0 | 100.00 |
| 6. | Empty HGC | 50.00 | NA | NA | NA | NA | NA |
| | Swedish orange | | | | | | |
| | Size 3 ACG | | | | | | |
| 7 | Empty HGC Dark | NA | | 50.00 | | NA | |
| | Green Size 3 ACG | | | | | | |
| 8 | Empty HGC | NA | | NA | | 90.0 | |
| | Brown opaque | | | | | | |
| | Size 0 ACG | | | | | | |
| | Total Capsule | 220.0 | | 220.0 | | 470.0 | |
| | weight | | | | | | |

Note:
Assay, water content and Residual solvent was incorporated for API calculation

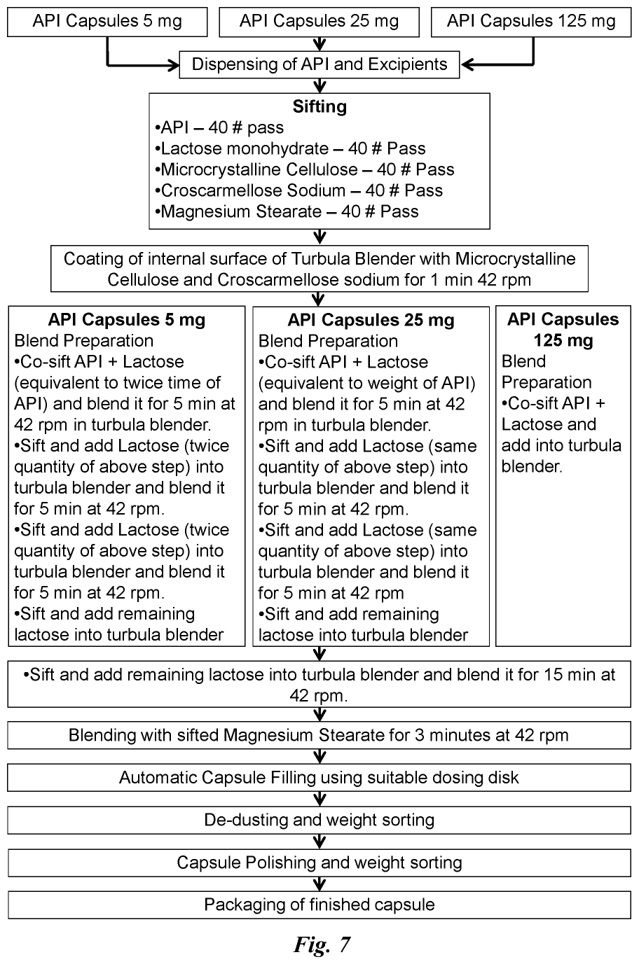

FIG. 7 is a block diagram illustration of a manufacturing process of the present disclosure. Capsules were filled by using automatic capsule filling machine. Over three batches, automated capsule machine was run at 80 rpm. In 1 minute, 80 capsules were filled with high accuracy. Physical parameters of finish dosage forms were within specification limits. No single capsule was rejected during weight sorting. The RSD value was less than 1.2%.

TABLE 31
Uniformity of dosage form and assay of pilot batches

| | Batch No | | |
|---|---|---|---|
| | 6363AG01 | 6363AH01 | 6363AI01 |
| | Uniformity of dosage | | Uniformity of dosage |
| | form by Chemical | | form by Weight |
| Sr. No. | Analysis | | variation |
| 1 | 94.5 | 98.6 | 98.6 |
| 2 | 94.9 | 98.7 | 99.3 |
| 3 | 96.5 | 96.2 | 98.7 |
| 4 | 95.8 | 97.0 | 99.4 |
| 5 | 95.3 | 99.7 | 99.0 |
| 6 | 94.0 | 99.2 | 99.5 |
| 7 | 94.7 | 98.3 | 99.5 |
| 8 | 97.2 | 101.4 | 99.9 |
| 9 | 96.2 | 98.4 | 98.7 |
| 10 | 95.7 | 102.4 | 99.3 |
| Avg | 95.5 | 99.0 | 99.2 |
| SD | 1.0 | 1.8 | 0.43 |
| RSD | 1.0 | 1.9 | 0.43 |
| Minimum | 94.0 | 96.2 | 98.6 |
| Maximum | 97.2 | 102.4 | 99.9 |
| AV Value | 5.4 | 4.4 | 1.0 |
| Assay 1 | 96.3 | 97.8 | 99.5 |
| Assay 2 | 96.7 | 98.6 | 98.8 |
| Average | 96.5 | 98.2 | 99.2 |
| Assay | | | |

Dissolution in pH 1.2 was then tested.

TABLE 32
Dissolution data in pH 1.2

| Dissolution Condition | 0.1N HCl (pH 1.2), 50 rpm, paddle with sinker, 900 ml | | | | | |
|---|---|---|---|---|---|---|
| Batch No | 6363AG01 | | 6363AH01 | | 6363AI01 | |
| Time | % Drug | % | % Drug | % | % Drug | % |
| Point | Released | RSD | Released | RSD | Released | RSD |
| 0 | 0 | 0 | 0 | 0 | 0 | 0 |
| 5 | 90 | 1.3 | 92 | 2.9 | 80 | 10.8 |
| 10 | 94 | 0.8 | 97 | 1.8 | 92 | 4.4 |
| 15 | 94 | 0.8 | 98 | 2.1 | 95 | 2.3 |
| 30 | 94 | 0.9 | 98 | 2.7 | 98 | 1.1 |
| 45 | 95 | 1.3 | 98 | 3.0 | 98 | 0.8 |

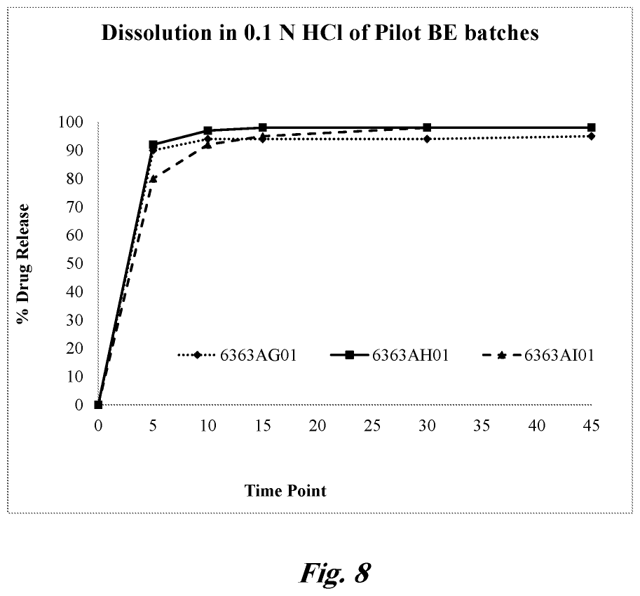

As illustrated in FIG. 8, a physical and chemical analysis of the capsules was found satisfactory and within targeted limits. More than 85% drug release was observed within 15 minutes in 0.1 N HCl in all strength. Uniformity of dosage unit (by chemical analysis for 5 mg and 25 mg and by weight variation for 125 mg) and assay values were within specification limits for all strength. The AV value of all the strengths was below 6.0. Batches were loaded on stability on accelerated condition (40° C./75% RH up to 6 months) and long term condition (25° C./60% RH up to 24 months) to evaluate the impact of storage condition on appearance, related substance, assay, dissolution and water content.

### Example 7—Final Formulations

The physical and chemical properties of capsules at initial and different stability condition were found satisfactory with respect to targeted limits. This formula and process was finalized.

TABLE 33
Final quantitative formula for API capsule: 5 mg, 25 mg, and 125 mg

| | | Standard | | Standard | | Standard | |
|---|---|---|---|---|---|---|---|
| Sr. | | Qty. | | Qty. | | Qty. | |
| No. | Ingredients | (mg)/Cap | % w/w | (mg)/Cap | % w/w | (mg)/Cap | % w/w |
| Capsules | | 5 mg | | 25 mg | | 125 mg | |
| 1. | Comp of formula | 5.400 | 3.18 | 27.00 | 15.88 | 135.0 | 35.53 |
| | (I) HCl (API) | | | | | | |
| | (# 40 sieve | | | | | | |
| | passed) | | | | | | |
| 2. | Microcrystalline | 36.80 | 21.65 | 31.50 | 18.53 | 53.10 | 13.97 |
| | Cellulose (Avicel | | | | | | |
| | PH102) | | | | | | |
| 3. | Lactose, | 121.0 | 71.18 | 104.7 | 61.59 | 176.7 | 46.50 |
| | Monohydrate, | | | | | | |
| | Compendial | | | | | | |
| | [Modified Spray | | | | | | |
| | Dried, 316 Fast | | | | | | |
| | Flow] | | | | | | |
| 4. | Croscarmellose | 5.100 | 3.000 | 5.100 | 3.000 | 11.40 | 3.000 |
| | Sodium (Ac-Di- | | | | | | |
| | Sol) | | | | | | |
| 5. | Magnesium | 1.700 | 1.000 | 1.700 | 1.000 | 3.800 | 1.000 |
| | Stearate (Non- | | | | | | |
| | bovine) | | | | | | |
| Total | | 170.0 | 100.0 | 170.0 | 100.00 | 380.0 | 100.00 |
| 6. | Empty HGC | 50.00 | NA | NA | NA | NA | NA |
| | Swedish orange | | | | | | |
| | Size 3 ACG | | | | | | |
| 7 | Empty HGC Dark | NA | | 50.00 | | NA | |
| | Green Size 3 | | | | | | |
| | ACG | | | | | | |
| 8 | Empty HGC | NA | | NA | | 90.0 | |
| | Brown opaque | | | | | | |
| | Size 0 ACG | | | | | | |
| | Total Capsule | 220.0 | | 220.0 | | 470.0 | |
| | weight | | | | | | |

TABLE 34
5 mg

| | Standard | | Standard | Adjusted |
|---|---|---|---|---|
| | Qty. | | Qty. | Qty. |
| Ingredients | (mg)/Cap | % w/w | (g)/batch | (g)/batch |
| API | 5.400 | 3.180 | 37.8 | 38.89* |
| (# 40 sieve passed) | | | | |
| Microcrystalline | 36.80 | 21.65 | 257.6 | 257.6 |
| Cellulose (Avicel | | | | |
| PH102) | | | | |
| (# 40 sieve passed) | | | | |
| Lactose, Monohydrate, | 121.0 | 71.18 | 847.0 | 845.9* |
| Compendial [Modified | | | | |
| Spray Dried, 316 Fast | | | | |
| Flow] (# 40 sieve | | | | |
| passed) | | | | |
| Croscarmellose Sodium | 5.100 | 3.000 | 35.70 | 35.70 |
| (Ac-Di-Sol) | | | | |
| (# 40 sieve passed) | | | | |
| Magnesium Stearate | 1.700 | 1.000 | 11.90 | 11.90 |
| (Non-bovine) (# 40 | | | | |
| sieve passed) | | | | |
| Total | 170.0 | 100.0 | 1190 | 1190 |

| | | |
|---|---|---|
| Empty HGC Swedish | 50.00# | NA |
| orange Size 3 ACG | | |
| Total Capsule weight | 220.0 | |

Note :
API Qty is adjusted on the basis of Assay and water content of API, which is compensated with lactose monohydrate.
# Average capsule weight will be finalized at the time of dispensing.

TABLE 35
25 mg

| | Standard | | Standard | Adjusted |
|---|---|---|---|---|
| | Qty. | | Qty. | Qty. |
| Ingredients | (mg)/Cap | % w/w | (g)/batch | (g)/batch |
| API | 27.00 | 15.88 | 189.0 | 194.5* |
| (# 40 sieve passed) | | | | |
| Microcrystalline | 31.50 | 18.53 | 220.5 | 220.5 |
| Cellulose (Avicel | | | | |
| PH102) | | | | |
| (# 40 sieve passed) | | | | |
| Lactose, Monohydrate, | 104.7 | 61.59 | 732.9 | 727.4* |
| Compendial [Modified | | | | |
| Spray Dried, 316 Fast | | | | |
| Flow] (# 40 sieve | | | | |
| passed) | | | | |
| Croscarmellose Sodium | 5.100 | 3.000 | 35.70 | 35.70 |
| (Ac-Di-Sol) | | | | |
| (# 40 sieve passed) | | | | |
| Magnesium Stearate | 1.700 | 1.000 | 11.90 | 11.90 |
| (Non-bovine) (# 40 sieve | | | | |
| passed) | | | | |
| Total | 170.0 | 100.0 | 1190 | 1190 |

| | | |
|---|---|---|
| Empty HGC Swedish | 50.00# | NA |
| orange Size 3 ACG | | |
| Total Capsule weight | 220.0 | |

Note:
API Qty is adjusted on the basis of Assay and water content of API, which is compensated with lactose monohydrate.
# Average capsule weight will be finalized at the time of dispensing.

TABLE 36
125 mg

| | Standard | | Standard | Adjusted |
|---|---|---|---|---|
| | Qty. | | Qty. | Qty. |
| Ingredients | (mg)/Cap | % w/w | (g)/batch | (g)/batch |
| API | 135.0* | 35.53 | 945.0 | 972.4* |
| (# 40 sieve passed) | | | | |
| Microcrystalline | 53.10 | 13.97 | 371.7 | 371.7 |
| Cellulose (Avicel | | | | |
| PH102) | | | | |
| (# 40 sieve passed) | | | | |
| Lactose, Monohydrate, | 176.7 | 46.50 | 1236.9 | 1209.5 |
| Compendial [Modified | | | | |
| Spray Dried, 316 Fast | | | | |
| Flow] (# 40 sieve | | | | |
| passed) | | | | |
| Croscarmellose Sodium | 11.40 | 3.000 | 79.80 | 79.80 |
| (Ac-Di-Sol) | | | | |
| (# 40 sieve passed) | | | | |
| Magnesium Stearate | 3.800 | 1.000 | 26.60 | 26.60 |
| (Non-bovine) (# 40 sieve | | | | |
| passed) | | | | |
| Total | 380.0 | 100.0 | 2660 | 2660 |

| | | |
|---|---|---|
| Empty HGC Swedish | 90 | NA |
| orange Size 3 ACG# | | |
| Total Capsule weight | 470.0 | |

Note:
API Qty is adjusted on the basis of Assay and water content of API, which is compensated with lactose monohydrate.
# Average capsule weight will be finalized at the time of dispensing.

All three capsules were subject to long term stability testing conditions at 25° C. and 60% relative humidity (RH) as well as 40° C. and 75% RH. All three capsules were shown to be stable for 24 months at a storage condition of 25° C./60% RH and stable for 6 months at a storage condition of 40° C./75% RH. The long-term stability data (percentage of total degradation products) for all three dose strengths under 25° C./60% RH is shown in Table 37 below.

TABLE 37
Percentage of total degradation products for capsules 5 mg,
25 mg, and 125 mg (25° C./60% RH)

| | 5 mg | 25 mg | 125 mg |
|---|---|---|---|
| Initial | 0.11% | 0.11% | 0.10% |
| 3 months | 0.10% | 0.09% | 0.06% |
| 6 months | 0.11% | 0.08% | 0.09% |
| 9 months | 0.11% | 0.10% | 0.08% |
| 12 months | 0.08% | — | 0.05% |
| 18 months | 0.08% | 0.10% | 0.08% |
| 24 months | 0.10% | 0.08% | 0.06% |

The long-term stability data (total degradation products) for all three dose strengths under 40° C./75% RH is shown in Table 38 below.

TABLE 38
Percentage of total degradation products for capsules 5 mg,
25 mg, and 125 mg (40° C./75% RH)

| | 5 mg | 25 mg | 125 mg |
|---|---|---|---|
| Initial | 0.11% | 0.11% | 0.10% |
| 1 month | 0.09% | 0.10% | 0.08% |
| 2 months | 0.11% | 0.13% | 0.07% |
| 3 months | 0.12% | 0.07% | 0.06% |
| 6 months | 0.10% | 0.09% | 0.09% |

In addition to the above-noted specific formulations, a disintegrant may be present in the composition in an amount of about 0.1% to about 30.0%. In a further embodiment, disintegrant may be present in an amount of about 0.5% to about 20.0%. When the disintegrant is croscarmellose, a preferred embodiment provides croscarmellose in an amount of about 0.1% to about 6.0%.

In addition to the above-noted specific formulations, a lubricant may be present in the composition in an amount of about 0.1% to about 5.0%. In a further embodiment, lubricant may be present in an amount of about 0.5% to about 3.0%. When the disintegrant is magnesium stearate, a preferred embodiment provides magnesium stearate in an amount of about 0.1% to about 3.0%. Other lubricants include, but are not limited to, SSF (Sodium Stearyl Fumarate) and SLS (Sodium Lauryl Sulfonate).

In addition to the above-noted specific formulations, one or more diluent or filler may be present in the composition in a combined amount of about 10% to about 80%. As described, one diluent or filler may be present in an amount of about 12% to about 25% and another diluent or filler may be present in amount of about 45% to about 75%. In one embodiment a preferred combination of diluents includes microcrystalline cellulose and lactose. Other diluents include, but are not limited to, Mannitol and Starch 1500.

Numbered embodiments of the present disclosure include:
1. An oral solid pharmaceutical composition comprising
   a. a compound of formula (I):

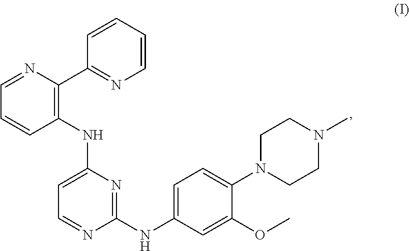 [SMILES: COc1cc(Nc2nccc(Nc3cccnc3-c3ccccn3)n2)ccc1N1CCN(C)CC1]

or a pharmaceutically acceptable salt thereof;
b. microcrystalline cellulose;
c. lactose;
d. croscarmellose; and
e. magnesium stearate.
2. The pharmaceutical composition of 1, wherein the compound of formula (I) or the pharmaceutically acceptable salt is in an amount from about 3 mg to about 350 mg, based on free base weight, wherein the pharmaceutical composition is a gelatin capsule.
3. The pharmaceutical composition of 1 or 2, wherein the pharmaceutically acceptable salt is a hydrochloric acid salt.
4. The pharmaceutical composition of any one of 1-3, wherein the gelatin capsule is (i) 5 mg, (ii) 25 mg, or (iii) 125 mg strength, based on free base weight.
5. The pharmaceutical composition of 3, wherein the gelatin capsule is (i) 30 mg, (ii) 60 mg, (iii) 90 mg, (iv) 120 mg, (v) 150 mg, (vi) 180 mg, (vii) 210 mg, (viii) 240 mg, (ix) 270 mg, or (x) 300 mg strength, based on free base weight.
6. The pharmaceutical composition of any one of 1-5, wherein the amount of microcrystalline cellulose is from about 0% w/w to about 50% w/w.
7. The pharmaceutical composition of 6, wherein the amount of microcrystalline cellulose is from about 10% w/w to about 25% w/w.
8. The pharmaceutical composition of 7, wherein the amount of microcrystalline cellulose is from about 13% w/w to about 23% w/w.
9. The pharmaceutical composition of 8, wherein the amount of microcrystalline cellulose is from about 14% w/w to about 22% w/w.
10. The pharmaceutical composition of any one of 1-9, wherein the amount of lactose is from about 10% w/w to about 80% w/w.
11. The pharmaceutical composition of 10, wherein the amount of lactose is from about 45% w/w to about 75% w/w.
12. The pharmaceutical composition of 11, wherein the amount of lactose is from about 46% w/w to about 72% w/w.
13. The pharmaceutical composition of 12, wherein the amount of lactose is from about 47% w/w to about 71% w/w.
14. The pharmaceutical composition of any one of 1-13, wherein the amount of croscarmellose is from about 0.1% w/w to about 6.0% w/w.
15. The pharmaceutical composition of 14, wherein the amount of croscarmellose is about 3.0% w/w.
16. The pharmaceutical composition of any one of 1-15, wherein the amount of magnesium stearate is from about 0.1% w/w to about 3.0% w/w.
17. The pharmaceutical composition of 16, wherein the amount of magnesium stearate is about 1.0% w/w.
18. An oral solid pharmaceutical composition comprising
   a. a compound of formula (I):

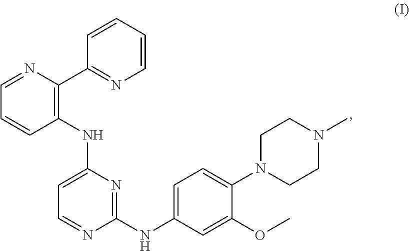 [SMILES: COc1cc(Nc2nccc(Nc3cccnc3-c3ccccn3)n2)ccc1N1CCN(C)CC1]

or a pharmaceutically acceptable salt thereof;
b. one or more diluent;
c. one or more disintegrant; and
d. one or more lubricant.
19. The pharmaceutical composition of 18, wherein the composition comprises a total amount of diluent in an amount of about 10% w/w to about 80% w/w.
20. The pharmaceutical composition of 18 or 19, wherein the composition comprises two different diluents.
21. The pharmaceutical composition of 20, wherein one diluent is present in an amount of about 12% to about 25% and another diluent is present in amount of about 45% to about 75%.
22. The pharmaceutical composition of any one of 18-21, wherein the one or more diluent is selected from microcrystalline cellulose, lactose, and combinations thereof.
23. The pharmaceutical composition of any one of 18-21, wherein the one or more disintegrant is present in an amount of about 0.1% w/w to about 30.0% w/w.
24. The pharmaceutical composition of 23, wherein the one or more disintegrant is present in an amount of about 0.5% w/w to about 20.0% w/w.
25. The pharmaceutical composition of 24, wherein the one or more disintegrant is present in an amount of about 0.1% w/w to about 6.0% w/w.
26. The pharmaceutical composition of 25, wherein the amount of disintegrant is about 3.0% w/w.
27. The pharmaceutical composition of any one of 18-26, wherein the disintegrant is croscarmellose sodium.
28. The pharmaceutical composition of any one of 18-27, wherein the one or more lubricant is present in an amount of about 0.1% w/w to about 5.0% w/w.
29. The pharmaceutical composition of 28, wherein the one or more lubricant is present in an amount of about 0.5% w/w to about 3.0% w/w.
30. The pharmaceutical composition of 28, wherein the one or more lubricant is present in an amount of about 0.1% w/w to about 3.0% w/w.
31. The pharmaceutical composition of 28, wherein the amount of lubricant is about 1.0% w/w.
32. The pharmaceutical composition of any one of 18-31, wherein the lubricant is magnesium stearate.
33. An oral solid pharmaceutical composition comprising:
   a. about 5.4 mg of a compound of formula (I):

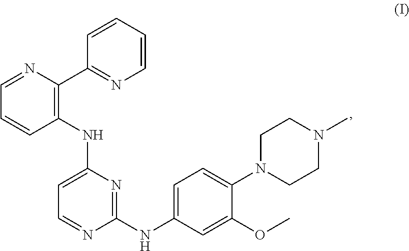 [SMILES: COc1cc(Nc2nccc(Nc3cccnc3-c3ccccn3)n2)ccc1N1CCN(C)CC1]

or a pharmaceutically acceptable salt thereof;
b. about 36.80 mg of microcrystalline cellulose;
c. about 121.00 mg of lactose;
d. about 5.10 mg of croscarmellose sodium; and
e. about 1.70 mg of magnesium stearate.
34. An oral solid pharmaceutical composition comprising:
   a. about 27.00 mg of a compound of formula (I):

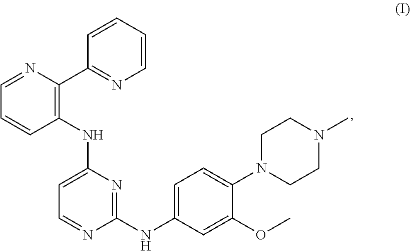 [SMILES: COc1cc(Nc2nccc(Nc3cccnc3-c3ccccn3)n2)ccc1N1CCN(C)CC1]

or a pharmaceutically acceptable salt thereof;
b. about 31.50 mg of microcrystalline cellulose;
c. about 104.70 mg of lactose;
d. about 5.10 mg of croscarmellose sodium; and
e. about 1.70 mg of magnesium stearate.
35. An oral solid pharmaceutical composition comprising:
   a. about 135.00 mg of a compound of formula (I):

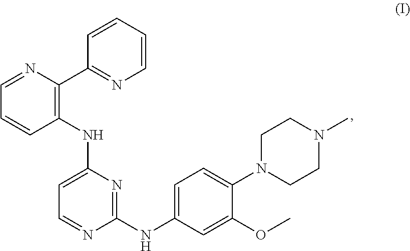 [SMILES: COc1cc(Nc2nccc(Nc3cccnc3-c3ccccn3)n2)ccc1N1CCN(C)CC1]

or a pharmaceutically acceptable salt thereof;
b. about 53.10 mg of microcrystalline cellulose;
c. about 176.70 mg of lactose;
d. about 11.40 mg of croscarmellose sodium; and
e. about 3.80 mg of magnesium stearate.
36. An oral solid pharmaceutical composition comprising:
   a. about 3.18% w/w of a compound of formula (I):

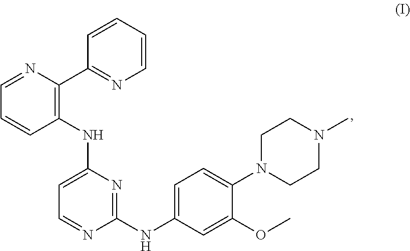 [SMILES: COc1cc(Nc2nccc(Nc3cccnc3-c3ccccn3)n2)ccc1N1CCN(C)CC1]

or a pharmaceutically acceptable salt thereof;
b. about 21.65% w/w of microcrystalline cellulose;
c. about 71.18% w/w of lactose;
d. about 3.00% w/w of croscarmellose sodium; and
e. about 1.00% w/w of magnesium stearate.
37. An oral solid pharmaceutical composition comprising:
   a. about 15.88% w/w of a compound of formula (I):

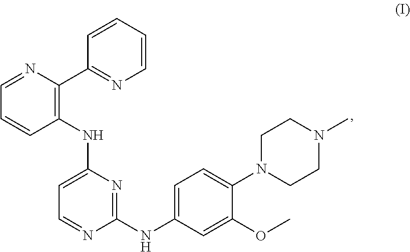 [SMILES: COc1cc(Nc2nccc(Nc3cccnc3-c3ccccn3)n2)ccc1N1CCN(C)CC1]

or a pharmaceutically acceptable salt thereof;
b. about 18.53% w/w of microcrystalline cellulose;
c. about 61.59% w/w of lactose;
d. about 3.00% w/w of croscarmellose sodium; and
e. about 1.00% w/w of magnesium stearate.
38. An oral solid pharmaceutical composition comprising:
   a. about 35.53% w/w of a compound of formula (I):

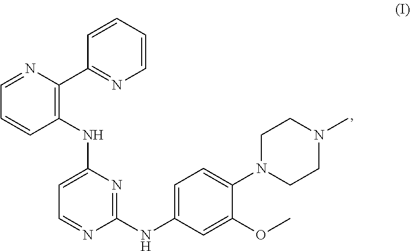 [SMILES: COc1cc(Nc2nccc(Nc3cccnc3-c3ccccn3)n2)ccc1N1CCN(C)CC1]

or a pharmaceutically acceptable salt thereof;
b. about 13.97% w/w of microcrystalline cellulose;
c. about 46.50% w/w of lactose;
d. about 3.00% w/w of croscarmellose sodium; and
e. about 1.00% w/w of magnesium stearate.
39. A method of treating a disease or disorder mediated by or associated with inhibition of one or more of ALK2 (ACVR1), JAK2, and ALK5 comprising administering a pharmaceutical composition of any one of 1-38.
40. A composition for use in medicine comprising the composition of any one of 1-38.
41. A composition of any one of 1-38 as a medicament for the treatment of a disease or disorder mediated by or associated with inhibition of one or more of ALK2 (ACVR1), JAK2, and ALK5.
42. Use of a composition of any one of 1-38, for the treatment of a disease or disorder mediated by or associated with inhibition of one or more of ALK2 (ACVR1), JAK2, and ALK5.
43. The pharmaceutical composition of any one of 1-38, wherein the pharmaceutically acceptable salt is a hydrochloric acid crystalline salt of the compound of formula (I).
44. The pharmaceutical composition of 43, wherein the pharmaceutically acceptable salt is Form A of the hydrochloric acid crystalline salt.
45. An oral solid pharmaceutical composition comprising:
   a. about 5.4 mg of hydrochloric acid salt of the compound of formula (I);
   b. about 36.80 mg of microcrystalline cellulose;
   c. about 121.00 mg of lactose;
   d. about 5.10 mg of croscarmellose sodium; and
   e. about 1.70 mg of magnesium stearate.
46. An oral solid pharmaceutical composition comprising:
   a. about 27.00 mg of hydrochloric acid salt of the compound of formula (I);
   b. about 31.50 mg of microcrystalline cellulose;
   c. about 104.70 mg of lactose;
   d. about 5.10 mg of croscarmellose sodium; and
   e. about 1.70 mg of magnesium stearate.
47. An oral solid pharmaceutical composition comprising:
   a. about 135.00 mg of hydrochloric acid salt of the compound of formula (I);
   b. about 53.10 mg of microcrystalline cellulose;
   c. about 176.70 mg of lactose;
   d. about 11.40 mg of croscarmellose sodium; and
   e. about 3.80 mg of magnesium stearate.
48. An oral solid pharmaceutical composition comprising:
   a. about 3.18% w/w of hydrochloric acid salt of the compound of formula (I);
   b. about 21.65% w/w of microcrystalline cellulose;
   c. about 71.18% w/w of lactose;
   d. about 3.00% w/w of croscarmellose sodium; and
   e. about 1.00% w/w of magnesium stearate.
49. An oral solid pharmaceutical composition comprising:
   a. about 15.88% w/w of hydrochloric acid salt of the compound of formula (I);
   b. about 18.53% w/w of microcrystalline cellulose;
   c. about 61.59% w/w of lactose;
   d. about 3.00% w/w of croscarmellose sodium; and
   e. about 1.00% w/w of magnesium stearate.
50. An oral solid pharmaceutical composition comprising:
   a. about 35.53% w/w of hydrochloric acid salt of the compound of formula (I);
   b. about 13.97% w/w of microcrystalline cellulose;
   c. about 46.50% w/w of lactose;
   d. about 3.00% w/w of croscarmellose sodium; and
   e. about 1.00% w/w of magnesium stearate.
51. The pharmaceutical composition of any one of 45-50, wherein the pharmaceutically acceptable salt is a hydrochloric acid crystalline salt of the compound of formula (I).
52. The pharmaceutical composition of 51, wherein the pharmaceutically acceptable salt is Form A of the hydrochloric acid crystalline salt.
53. An oral solid pharmaceutical composition comprising
   about 3 mg to about 350 mg of a compound of formula (I) or a pharmaceutically acceptable salt thereof;
   one or more diluent;
   one or more disintegrant; and
   one or more lubricant.
54. An oral solid pharmaceutical composition comprising
   about 3 mg to about 150 mg of a compound of formula (I) or a pharmaceutically acceptable salt thereof;
   about 30 mg to 260 mg of one or more diluent;
   about 3 mg to about 13 mg of one or more disintegrant; and
   about 1 mg to about 5 mg of one or more lubricant.
55. An oral solid pharmaceutical composition comprising
   about 1-50% w/w of a compound of formula (I) or a pharmaceutically acceptable salt thereof;
   about 10-95% w/w of one or more diluent;
   about 0.1-6.0% w/w of one or more disintegrant; and
   about 0.1-3.0% w/w of one or more lubricant.
56. An oral solid pharmaceutical composition comprising
   about 2-38% w/w of a compound of formula (I) or a pharmaceutically acceptable salt thereof;
   about 10-80% w/w of one or more diluent;
   about 2-4% w/w of one or more disintegrant; and about 0.7-1.3% w/w of one or more lubricant.
57. An oral solid pharmaceutical composition comprising:
   about 3 mg to about 30 mg of a compound of formula (I), or a pharmaceutically acceptable salt thereof;
   about 30 mg to about 60 mg of microcrystalline cellulose;
   about 100 mg to about 200 mg of lactose;
   about 3 mg to about 13 mg of croscarmellose sodium; and
   about 1 mg to about 5 mg of magnesium stearate.
58. An oral solid pharmaceutical composition comprising:
   about 5 mg to 6 mg of a compound of formula (I), or a pharmaceutically acceptable salt thereof;
   about 33 mg to 39 mg microcrystalline cellulose;
   about 115 mg to 125 mg lactose;
   about 4 mg to 6 mg croscarmellose sodium; and
   about 1.4 mg to 2 mg magnesium stearate.
59. An oral solid pharmaceutical composition comprising:
   about 25.00 mg to 30 mg of a compound of formula (I), or a pharmaceutically acceptable salt thereof;
   about 28 mg to 35 mg of microcrystalline cellulose;
   about 100 mg to 110 mg of lactose;
   about 4 mg to 6 mg of croscarmellose sodium; and
   about 1.4 mg to 2 mg of magnesium stearate.
60. An oral solid pharmaceutical composition comprising:
   about 125.00 mg to 140 mg of a compound of formula (I), or a pharmaceutically acceptable salt thereof;
   about 48 mg to 58 mg of microcrystalline cellulose;
   about 170 mg to 180 mg of lactose;
   about 8 mg to 14 mg of croscarmellose sodium; and
   about 3.3 mg to 4.3 mg of magnesium stearate.
61. An oral solid pharmaceutical composition comprising:
   about 2-38% w/w of a compound of formula (I) or a pharmaceutically acceptable salt thereof;
   about 12-25% w/w of microcrystalline cellulose;
   about 45-75% w/w of lactose;
   about 2-4% w/w of croscarmellose sodium; and
   about 0.7-1.3% w/w of magnesium stearate.
62. An oral solid pharmaceutical composition comprising:
   about 2-38% w/w of a compound of formula (I) or a pharmaceutically acceptable salt thereof;
   about 12-25% w/w of microcrystalline cellulose;
   about 45-75% w/w of lactose;
   about 3% w/w of croscarmellose sodium; and
   about 1% w/w of magnesium stearate.
63. The pharmaceutical composition of any one of 53-62, wherein the pharmaceutically acceptable salt is a hydrochloric acid crystalline salt of the compound of formula (I).
64. The pharmaceutical composition of 63, wherein the pharmaceutically acceptable salt is Form A of the hydrochloric acid crystalline salt.
65. An oral solid pharmaceutical composition comprising:
   about 3-3.3% w/w of hydrochloric acid salt of the compound of formula (I);
   about 20-23% w/w of microcrystalline cellulose;
   about 70-73% w/w of lactose;
   about 3.00% w/w of croscarmellose sodium; and
   about 1.00% w/w of magnesium stearate.
66. An oral solid pharmaceutical composition comprising:
   about 14-17% w/w of hydrochloric acid salt of the compound of formula (I);
   about 17-20% w/w of microcrystalline cellulose;
   about 60-64% w/w of lactose;
   about 3.00% w/w of croscarmellose sodium; and
   about 1.00% w/w of magnesium stearate.
67. An oral solid pharmaceutical composition comprising:
   about 34-37% w/w of hydrochloric acid salt of the compound of formula (I);
   about 12-15% w/w of microcrystalline cellulose;
   about 45-48% w/w of lactose;
   about 3.00% w/w of croscarmellose sodium; and
   about 1.00% w/w of magnesium stearate.
68. A method for treating cancer, anemia or anemia related conditions, or myelodysplastic syndrome (MDS) in a subject comprising administering to the subject in need thereof an effective amount of a compound of formula (I):

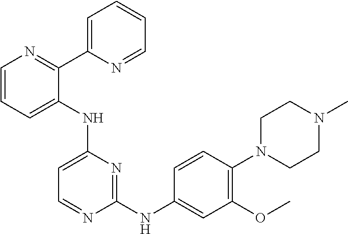 [SMILES: COc1cc(Nc2nccc(Nc3cccnc3-c3ccccn3)n2)ccc1N1CCN(C)CC1]

or a pharmaceutically acceptable salt thereof;
wherein the compound of formula (I) is formulated in a pharmaceutical composition according to any one of 1-67.
69. The method of 68, wherein the anemia is anemia of chronic disease, anemia of chronic inflammation, anemia associated with cancer or fibrodysplasia ossificans progressive.
70. The method of 68, wherein the subject has very low, low or intermediate myelodysplastic syndrome.
71. The method of 68, wherein the anemia is anemia associated with MDS, transfusion dependent anemia associated with MDS, MDS with single lineage dysplasia refractory anemia, or MDS with ring sideroblasts.
72. The method of 68, wherein the MDS is primary MDS, secondary MDS, high-risk MDS, intermediate-risk MDS, low-risk MDS, very low, low or intermediate MDS.
73. The method of 68, wherein the subject is intolerant, resistant, or refractory to luspatercept.
74. The method of 68, wherein the cancer is a breast cancer, ovarian cancer, prostate cancer, pancreatic cancer, or head and neck cancer.
75. The method of 68, wherein the cancer is a myeloproliferative disorder, a hematological cancer, or a solid tumor.
76. The method of 75, wherein the solid tumor is a breast tumor, ovarian tumor, prostate tumor, pancreatic tumor, or head and neck tumor.
77. The method of 75, wherein the solid tumor is renal cell carcinoma, or hepatocellular carcinoma.
78. The method of 75, wherein the myeloproliferative disorder is myelofibrosis, polycythemia vera, or essential thrombocytosis.
79. The method of 75, wherein the hematological cancer is lymphoma.
80. The method of 68, wherein the method improves one or more hematologic parameters in the subject, said improvement comprises decreasing myoblasts, increasing hemoglobin, increasing platelets, increasing neutrophils, decreasing hepcidin, reducing units of red blood cell transfused, reducing frequency of transfusion, and/or reducing transfusion dependence.
81. The method of 80, wherein the subject is suffering from anemia and has very low, low or intermediate myelodysplastic syndrome (MDS).
82. The method of 80, wherein units of red blood cells transfused is reduced by 4 or more units compared to the units of red blood cells transfused for the same period of time prior to administration of the compound of formula (I) or the pharmaceutically acceptable salt; wherein the period of time is 8 weeks or 12 weeks.
83. The method of 80, wherein the subject has transfusion dependent anemia associated with MDS, MDS with single lineage dysplasia refractory anemia, or MDS with ring sideroblasts and is intolerant, resistant or refractory to luspatercept.
84. The method of 80, wherein increasing hemoglobin is defined as increasing hemoglobin i) to 10 g/dL or more; or ii) by 1.5 g/dL or more compared to an amount measured prior to administration of the compound of formula (I) or the pharmaceutically acceptable salt;
   wherein the increase in hemoglobin is maintained for 8 weeks or 12 weeks in the absence of red blood cell transfusions.
85. The method of 80, wherein increasing platelets is defined as increasing the platelet count i) by 30×10⁹/L or more; or ii) to 75×10⁹/L or more; wherein the increase in platelets is maintained for 8 weeks or 12 weeks in the absence of red blood cell transfusions.
86. The method of 80, wherein increasing neutrophils is defined as increasing the neutrophil count i) by 0.5×10⁹/L or more or ii) to 1.0×10⁹/L or more; wherein the increase in neutrophil count is maintained for 8 weeks or 12 weeks in the absence of red blood cell transfusions.
87. The method of 80, wherein decreasing hepcidin is defined as decreasing hepcidin by 25% or more compared to a baseline amount measured prior to administration of the compound of formula (I) or the pharmaceutically acceptable salt.
88. The method of 80, wherein decreasing myoblasts is defined as decreasing myoblasts i) to be 5% or fewer of bone marrow cells; or ii) by 50% or more compared to a baseline amount measured prior to administration of the compound of formula (I) or the pharmaceutically acceptable salt.
89 The method of any one of 68-88, wherein the compound of formula (I) or the pharmaceutically acceptable salt is administered at a daily dosage of from 10 mg to 350 mg, from 90 mg to 120 mg, 20 mg, 40 mg, 60 mg, 90 mg, 120 mg, 160 mg, 210 mg, 270 mg, or 300 mg.
90. The method of any one of 68-88, wherein the compound of formula (I) or the pharmaceutically acceptable salt is administered at a maintenance dosage regime, wherein the maintenance dose is the dose at which the subject achieves and maintains for a period of time a predetermined threshold level of a hemoglobin or a biomarker.
91. The method of 90, wherein the biomarker is selected from
   i) hepcidin in serum and bone marrow aspirate;
   ii) iron metabolism markers in serum selected from iron, ferritin, transferrin, soluble transferrin receptor [STR], and total iron binding capacity [TIBC]; iii) cytokines in serum or plasma selected from CRP, EPO, IL-6, and TGF-beta 1; and
   iv) indicators of inhibition of signal transduction pathways in bone marrow aspirates selected from phosphorylation of SMAD-1, 2, 3, 5 and 8 in PBMCs.
92. The method of any one of 68-91, wherein the compound of formula (I) or the pharmaceutically acceptable salt is formulated in a gelatin capsule.
93. The method of 92, wherein the gelatin capsule comprises about 5 mg, 25 mg, or 125 mg of the compound of formula or the pharmaceutically acceptable salt, which is based on free base weight of the compound of formula (I).
94. The method of 92, wherein the gelatin capsule comprises:
   a) about 5.4 mg of hydrochloric acid salt of the compound of formula (I);
   b) about 36.80 mg of microcrystalline cellulose;
   c) about 121.00 mg of lactose;
   d) about 5.10 mg of croscarmellose sodium; and
   e) about 1.70 mg of magnesium stearate.
95. The method of 92, wherein the gelatin capsule comprises:
   a) about 27.00 mg of hydrochloric acid salt of the compound of formula (I);
   b) about 31.50 mg of microcrystalline cellulose;
   c) about 104.70 mg of lactose;
   d) about 5.10 mg of croscarmellose sodium; and
   e) about 1.70 mg of magnesium stearate.
96. The method of 92, wherein the gelatin capsule comprises:
   a) about 135.00 mg of hydrochloric acid salt of the compound of formula (I);
   b) about 53.10 mg of microcrystalline cellulose;
   c) about 176.70 mg of lactose;
   d) about 11.40 mg of croscarmellose sodium; and
   e) about 3.80 mg of magnesium stearate.
97. The method of 92, wherein the gelatin capsule comprises:
   a) about 3.18% w/w of hydrochloric acid salt of the compound of formula (I);
   b) about 21.65% w/w of microcrystalline cellulose;
   c) about 71.18% w/w of lactose;
   d) about 3.00% w/w of croscarmellose sodium; and
   e) about 1.00% w/w of magnesium stearate.
98. The method of 92, wherein the gelatin capsule comprises:
   a) about 15.88% w/w of hydrochloric acid salt of the compound of formula (I);
   b) about 18.53% w/w of microcrystalline cellulose;
   c) about 61.59% w/w of lactose;
   d) about 3.00% w/w of croscarmellose sodium; and
   e) about 1.00% w/w of magnesium stearate.
99. The method of 92, wherein the gelatin capsule comprises:
   a) about 35.53% w/w of hydrochloric acid salt of the compound of formula (I);
   b) about 13.97% w/w of microcrystalline cellulose;
   c) about 46.50% w/w of lactose;
   d) about 3.00% w/w of croscarmellose sodium; and
   e) about 1.00% w/w of magnesium stearate.
100. The method of 92, wherein the gelatin capsule comprises:
   a) about 3-3.3% w/w of hydrochloric acid salt of the compound of formula (I);
   b) about 20-23% w/w of microcrystalline cellulose;
   c) about 70-73% w/w of lactose;
   d) about 3.00% w/w of croscarmellose sodium; and
   e) about 1.00% w/w of magnesium stearate.
101. The method of 92, wherein the gelatin capsule comprises:
   a) about 14-17% w/w of hydrochloric acid salt of the compound of formula (I);
   b) about 17-20% w/w of microcrystalline cellulose;
   c) about 60-64% w/w of lactose;
   d) about 3.00% w/w of croscarmellose sodium; and
   e) about 1.00% w/w of magnesium stearate.
102. The method of 92, wherein the gelatin capsule comprises:
   a) about 34-37% w/w of hydrochloric acid salt of the compound of formula (I);
   b) about 12-15% w/w of microcrystalline cellulose;
   c) about 45-48% w/w of lactose;
   d) about 3.00% w/w of croscarmellose sodium; and
   e) about 1.00% w/w of magnesium stearate.
103. The method of any one of 68-102, wherein the pharmaceutically acceptable salt or hydrochloric acid salt is hydrochloric acid crystalline salt of the compound of formula (I).
104. The method of claim 103, wherein hydrochloric acid crystalline salt is Form A of the hydrochloric acid crystalline salt.

Each of the various embodiments described throughout this disclosure may be combined to provide further embodiments.

All U.S. patents, U.S. patent application publications, U.S. patent applications, non-U.S. patents, non-U.S. patent applications, and non-patent publications referred to in this specification and/or listed in the Application Data Sheet are incorporated herein by reference, in their entirety. U.S. Provisional Patent Application No. 62/939,489, filed Nov. 22, 2019, to which the present application claims priority, is hereby incorporated herein by reference in its entirety. Aspects of the embodiments may be modified, if necessary, to employ concepts of the various patents, applications, and publications to provide yet further embodiments.

Test compounds for the experiments described herein were employed in free or salt form, as noted.

The specific responses observed may vary according to and depending on the type of formulation and mode of administration employed, and such expected variations or differences in the results are contemplated in accordance with practice of the present invention.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

We claim:

1. An oral solid pharmaceutical composition comprising:
a) a compound of formula (I): 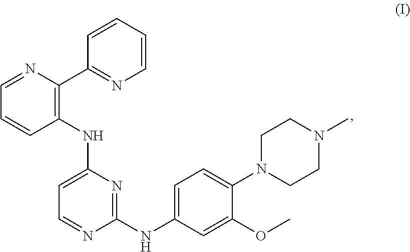 [SMILES: COc1cc(Nc2nccc(Nc3cccnc3-c3ccccn3)n2)ccc1N1CCN(C)CC1]
or a pharmaceutically acceptable salt thereof;
b) microcrystalline cellulose;
c) lactose;
d) croscarmellose; and
e) magnesium stearate.

2. The pharmaceutical composition of claim 1, wherein the compound of formula (I) or the pharmaceutically acceptable salt is in an amount from about 3 mg to about 350 mg based on free base weight, wherein the pharmaceutical composition is a gelatin capsule.

3. The pharmaceutical composition of claim 1 or 2, wherein the pharmaceutically acceptable salt is a hydrochloric acid salt.

4. The pharmaceutical composition of any one of claims 1-3, wherein the gelatin capsule is (i) 5 mg, (ii) 25 mg, or (iii) 125 mg strength, based on free base weight.

5. The pharmaceutical composition of claim 3, wherein the gelatin capsule is (i) 30 mg, (ii) 60 mg, (iii) 90 mg, (iv) 120 mg, (v) 150 mg, (vi) 180 mg, (vii) 210 mg, (viii) 240 mg, (ix) 270 mg, or (x) 300 mg strength, based on free base weight.

6. The pharmaceutical composition of any one of claims 1-5, wherein the amount of microcrystalline cellulose is from about 0% w/w to about 50% w/w.

7. The pharmaceutical composition of claim 6, wherein the amount of microcrystalline cellulose is from about 10% w/w to about 25% w/w.

8. The pharmaceutical composition of claim 7, wherein the amount of microcrystalline cellulose is from about 13% w/w to about 23% w/w.

9. The pharmaceutical composition of claim 8, wherein the amount of microcrystalline cellulose is from about 14% w/w to about 22% w/w.

10. The pharmaceutical composition of any one of claims 1-9, wherein the amount of lactose is from about 10% w/w to about 80% w/w.

11. The pharmaceutical composition of claim 10, wherein the amount of lactose is from about 45% w/w to about 75% w/w.

12. The pharmaceutical composition of claim 11, wherein the amount of lactose is from about 46% w/w to about 72% w/w.

13. The pharmaceutical composition of claim 12, wherein the amount of lactose is from about 47% w/w to about 71% w/w.

14. The pharmaceutical composition of any one of claims 1-13, wherein the amount of croscarmellose is from about 0.1% w/w to about 6.0% w/w.

15. The pharmaceutical composition of claim 14, wherein the amount of croscarmellose is about 3.0% w/w.

16. The pharmaceutical composition of any one of claims 1-15, wherein the amount of magnesium stearate is from about 0.1% w/w to about 3.0% w/w.

17. The pharmaceutical composition of claim 16, wherein the amount of magnesium stearate is about 1.0% w/w.

18. An oral solid pharmaceutical composition comprising:
a) a compound of formula (I): 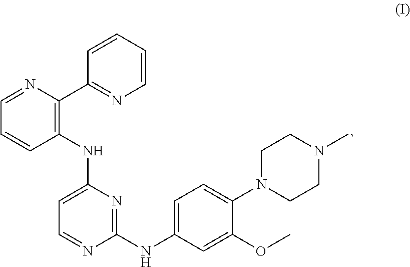 [SMILES: COc1cc(Nc2nccc(Nc3cccnc3-c3ccccn3)n2)ccc1N1CCN(C)CC1]
or a pharmaceutically acceptable salt thereof;
b) one or more diluent;
c) one or more disintegrant; and
d) one or more lubricant;
wherein the compound of formula (I) or the pharmaceutically acceptable salt is in an amount from about 3 mg to about 350 mg based on free base weight.

19. The pharmaceutical composition of claim 18, wherein the composition comprises a total amount of diluent in an amount of about 10% w/w to about 80% w/w.

20. The pharmaceutical composition of claim 18 or 19, wherein the composition comprises two different diluents.

21. The pharmaceutical composition of claim 20, wherein one diluent is present in an amount of about 12% to about 25% and another diluent is present in amount of about 45% to about 75%.

22. The pharmaceutical composition of any one of claims 18-21, wherein the one or more diluent is selected from microcrystalline cellulose, lactose, and combinations thereof.

23. The pharmaceutical composition of any one of claims 18-21, wherein the one or more disintegrant is present in an amount of about 0.1% w/w to about 30.0% w/w.

24. The pharmaceutical composition of claim 23, wherein the one or more disintegrant is present in an amount of about 0.5% w/w to about 20.0% w/w.

25. The pharmaceutical composition of claim 24, wherein the one or more disintegrant is present in an amount of about 0.1% w/w to about 6.0% w/w.

26. The pharmaceutical composition of claim 25, wherein the amount of disintegrant is about 3.0% w/w.

27. The pharmaceutical composition of any one of claims 18-26, wherein the disintegrant is croscarmellose sodium.

28. The pharmaceutical composition of any one of claims 18-27, wherein the one or more lubricant is present in an amount of about 0.1% w/w to about 5.0% w/w.

29. The pharmaceutical composition of claim 28, wherein the one or more lubricant is present in an amount of about 0.5% w/w to about 3.0% w/w.

30. The pharmaceutical composition of claim 28, wherein the one or more lubricant is present in an amount of about 0.1% w/w to about 3.0% w/w.

31. The pharmaceutical composition of claim 28, wherein the amount of lubricant is about 1.0% w/w.

32. The pharmaceutical composition of any one of claims 18-31, wherein the lubricant is magnesium stearate.

33. An oral solid pharmaceutical composition comprising:
a) about 5.4 mg of a compound of formula (I): 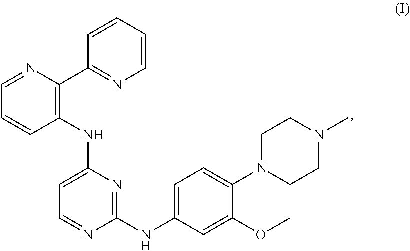 [SMILES: COc1cc(Nc2nccc(Nc3cccnc3-c3ccccn3)n2)ccc1N1CCN(C)CC1]
or a pharmaceutically acceptable salt thereof;
b) about 36.80 mg of microcrystalline cellulose;
c) about 121.00 mg of lactose;
d) about 5.10 mg of croscarmellose sodium; and
e) about 1.70 mg of magnesium stearate.

34. An oral solid pharmaceutical composition comprising:
a) about 27.00 mg of a compound of formula (I): 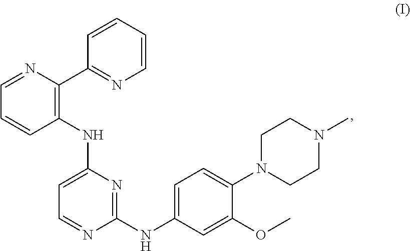 [SMILES: COc1cc(Nc2nccc(Nc3cccnc3-c3ccccn3)n2)ccc1N1CCN(C)CC1]
or a pharmaceutically acceptable salt thereof;
b) about 31.50 mg of microcrystalline cellulose;
c) about 104.70 mg of lactose;
d) about 5.10 mg of croscarmellose sodium; and
e) about 1.70 mg of magnesium stearate.

35. An oral solid pharmaceutical composition comprising:
a) about 135.00 mg of a compound of formula (I): 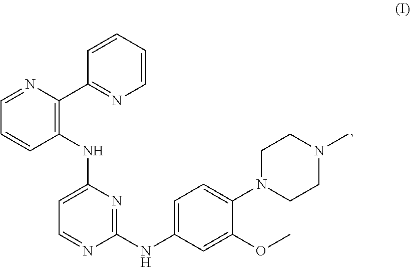 [SMILES: COc1cc(Nc2nccc(Nc3cccnc3-c3ccccn3)n2)ccc1N1CCN(C)CC1]
or a pharmaceutically acceptable salt thereof;
b) about 53.10 mg of microcrystalline cellulose;
c) about 176.70 mg of lactose;
d) about 11.40 mg of croscarmellose sodium; and
e) about 3.80 mg of magnesium stearate.

36. An oral solid pharmaceutical composition comprising:
a) about 3.18% w/w of a compound of formula (I): 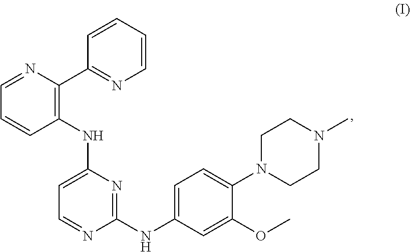 [SMILES: COc1cc(Nc2nccc(Nc3cccnc3-c3ccccn3)n2)ccc1N1CCN(C)CC1]
or a pharmaceutically acceptable salt thereof;
b) about 21.65% w/w of microcrystalline cellulose;
c) about 71.18% w/w of lactose;
d) about 3.00% w/w of croscarmellose sodium; and
e) about 1.00% w/w of magnesium stearate.

37. An oral solid pharmaceutical composition comprising:
a) about 15.88% w/w of a compound of formula (I): 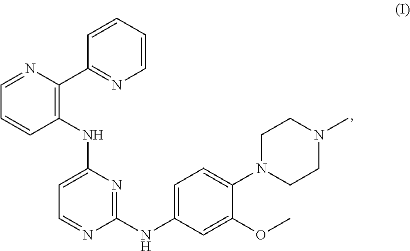 [SMILES: COc1cc(Nc2nccc(Nc3cccnc3-c3ccccn3)n2)ccc1N1CCN(C)CC1]
or a pharmaceutically acceptable salt thereof;
b) about 18.53% w/w of microcrystalline cellulose;
c) about 61.59% w/w of lactose;
d) about 3.00% w/w of croscarmellose sodium; and
e) about 1.00% w/w of magnesium stearate.

38. An oral solid pharmaceutical composition comprising:
a) about 35.53% w/w of a compound of formula (I): 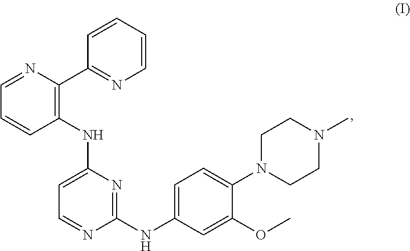 [SMILES: COc1cc(Nc2nccc(Nc3cccnc3-c3ccccn3)n2)ccc1N1CCN(C)CC1]
or a pharmaceutically acceptable salt thereof;
b) about 13.97% w/w of microcrystalline cellulose;
c) about 46.50% w/w of lactose;
d) about 3.00% w/w of croscarmellose sodium; and
e) about 1.00% w/w of magnesium stearate.

39. A method of treating a disease or disorder mediated by or associated with inhibition of one or more of ALK2, JAK2, and ALK5 comprising administering a pharmaceutical composition of any one of claims 1-38.

40. A composition for use in medicine comprising the composition of any one of claims 1-38.

41. A composition of any one of claims 1-38 as a medicament for the treatment of a disease or disorder mediated by or associated with inhibition of one or more of ALK2, JAK2, and ALK5.

42. Use of a composition of any one of claims 1-38, for the treatment of a disease or disorder mediated by or associated with inhibition of one or more of ALK2, JAK2, and ALK5.

43. The pharmaceutical composition of any one of claims 1-38, wherein the pharmaceutically acceptable salt is a hydrochloric acid crystalline salt of the compound of formula (I).

44. The pharmaceutical composition of claim 43, wherein the pharmaceutically acceptable salt is Form A of the hydrochloric acid crystalline salt.

45. An oral solid pharmaceutical composition comprising:
a) about 5.4 mg of hydrochloric acid salt of the compound of formula (I);
b) about 36.80 mg of microcrystalline cellulose;
c) about 121.00 mg of lactose;
d) about 5.10 mg of croscarmellose sodium; and
e) about 1.70 mg of magnesium stearate.

46. An oral solid pharmaceutical composition comprising:
a) about 27.00 mg of hydrochloric acid salt of the compound of formula (I);
b) about 31.50 mg of microcrystalline cellulose;
c) about 104.70 mg of lactose;
d) about 5.10 mg of croscarmellose sodium; and
e) about 1.70 mg of magnesium stearate.

47. An oral solid pharmaceutical composition comprising:
a) about 135.00 mg of hydrochloric acid salt of the compound of formula (I);
b) about 53.10 mg of microcrystalline cellulose;
c) about 176.70 mg of lactose;
d) about 11.40 mg of croscarmellose sodium; and
e) about 3.80 mg of magnesium stearate.

48. An oral solid pharmaceutical composition comprising:
a) about 3.18% w/w of hydrochloric acid salt of the compound of formula (I);
b) about 21.65% w/w of microcrystalline cellulose;
c) about 71.18% w/w of lactose;
d) about 3.00% w/w of croscarmellose sodium; and
e) about 1.00% w/w of magnesium stearate.

49. An oral solid pharmaceutical composition comprising:
a) about 15.88% w/w of hydrochloric acid salt of the compound of formula (I);
b) about 18.53% w/w of microcrystalline cellulose;
c) about 61.59% w/w of lactose;
d) about 3.00% w/w of croscarmellose sodium; and
e) about 1.00% w/w of magnesium stearate.

50. An oral solid pharmaceutical composition comprising:
a) about 35.53% w/w of hydrochloric acid salt of the compound of formula (I);
b) about 13.97% w/w of microcrystalline cellulose;
c) about 46.50% w/w of lactose;
d) about 3.00% w/w of croscarmellose sodium; and
e) about 1.00% w/w of magnesium stearate.

51. The pharmaceutical composition of any one of claims 45-50, wherein the pharmaceutically acceptable salt is a hydrochloric acid crystalline salt of the compound of formula (I).

52. The pharmaceutical composition of claim 51, wherein the pharmaceutically acceptable salt is Form A of the hydrochloric acid crystalline salt.

53. An oral solid pharmaceutical composition comprising
about 3 mg to about 350 mg of a compound of formula (I) or a pharmaceutically acceptable salt thereof;
one or more diluent;
one or more disintegrant; and
one or more lubricant.

54. An oral solid pharmaceutical composition comprising
about 3 mg to about 150 mg of a compound of formula (I) or a pharmaceutically acceptable salt thereof;
about 30 mg to 260 mg of one or more diluent;
about 3 mg to about 13 mg of one or more disintegrant; and
about 1 mg to about 5 mg of one or more lubricant.

55. An oral solid pharmaceutical composition comprising
about 1-50% w/w of a compound of formula (I) or a pharmaceutically acceptable salt thereof;
about 10-95% w/w of one or more diluent;
about 0.1-6.0% w/w of one or more disintegrant; and
about 0.1-3.0% w/w of one or more lubricant.

56. An oral solid pharmaceutical composition comprising
about 2-38% w/w of a compound of formula (I) or a pharmaceutically acceptable salt thereof;
about 10-80% w/w of one or more diluent;
about 2-4% w/w of one or more disintegrant; and
about 0.7-1.3% w/w of one or more lubricant.

57. An oral solid pharmaceutical composition comprising:
about 3 mg to about 30 mg of a compound of formula (I), or a pharmaceutically acceptable salt thereof;
about 30 mg to about 60 mg of microcrystalline cellulose;
about 100 mg to about 200 mg of lactose;
about 3 mg to about 13 mg of croscarmellose sodium; and
about 1 mg to about 5 mg of magnesium stearate.

58. An oral solid pharmaceutical composition comprising:
about 5 mg to 6 mg of a compound of formula (I), or a pharmaceutically acceptable salt thereof;
about 33 mg to 39 mg microcrystalline cellulose;
about 115 mg to 125 mg lactose;
about 4 mg to 6 mg croscarmellose sodium; and
about 1.4 mg to 2 mg magnesium stearate.

59. An oral solid pharmaceutical composition comprising:
about 25.00 mg to 30 mg of a compound of formula (I), or a pharmaceutically acceptable salt thereof;
about 28 mg to 35 mg of microcrystalline cellulose;
about 100 mg to 110 mg of lactose;
about 4 mg to 6 mg of croscarmellose sodium; and
about 1.4 mg to 2 mg of magnesium stearate.

60. An oral solid pharmaceutical composition comprising:
about 125.00 mg to 140 mg of a compound of formula (I), or a pharmaceutically acceptable salt thereof;
about 48 mg to 58 mg of microcrystalline cellulose;
about 170 mg to 180 mg of lactose;
about 8 mg to 14 mg of croscarmellose sodium; and
about 3.3 mg to 4.3 mg of magnesium stearate.

61. An oral solid pharmaceutical composition comprising:
about 2-38% w/w of a compound of formula (I) or a pharmaceutically acceptable salt thereof;
about 12-25% w/w of microcrystalline cellulose;
about 45-75% w/w of lactose;
about 2-4% w/w of croscarmellose sodium; and
about 0.7-1.3% w/w of magnesium stearate.

62. An oral solid pharmaceutical composition comprising:
about 2-38% w/w of a compound of formula (I) or a pharmaceutically acceptable salt thereof;
about 12-25% w/w of microcrystalline cellulose;
about 45-75% w/w of lactose;
about 3% w/w of croscarmellose sodium; and
about 1% w/w of magnesium stearate.

63. The pharmaceutical composition of any one of claims 53-62, wherein the pharmaceutically acceptable salt is a hydrochloric acid crystalline salt of the compound of formula (I).

64. The pharmaceutical composition of claim 63, wherein the pharmaceutically acceptable salt is Form A of the hydrochloric acid crystalline salt.

65. An oral solid pharmaceutical composition comprising:
about 3-3.3% w/w of hydrochloric acid salt of the compound of formula (I);
about 20-23% w/w of microcrystalline cellulose;
about 70-73% w/w of lactose;
about 3.00% w/w of croscarmellose sodium; and
about 1.00% w/w of magnesium stearate.

66. An oral solid pharmaceutical composition comprising:
about 14-17% w/w of hydrochloric acid salt of the compound of formula (I);
about 17-20% w/w of microcrystalline cellulose;
about 60-64% w/w of lactose;
about 3.00% w/w of croscarmellose sodium; and
about 1.00% w/w of magnesium stearate.

67. An oral solid pharmaceutical composition comprising:
about 34-37% w/w of hydrochloric acid salt of the compound of formula (I);
about 12-15% w/w of microcrystalline cellulose;
about 45-48% w/w of lactose;
about 3.00% w/w of croscarmellose sodium; and
about 1.00% w/w of magnesium stearate.

68. A method for treating cancer, anemia or anemia related conditions, or myelodysplastic syndrome (MDS) in a subject comprising administering to the subject in need thereof an effective amount of a compound of formula (I): 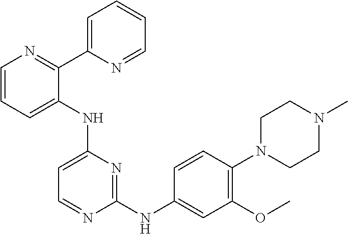 [SMILES: COc1cc(Nc2nccc(Nc3cccnc3-c3ccccn3)n2)ccc1N1CCN(C)CC1]
or a pharmaceutically acceptable salt thereof;
wherein the compound of formula (I) or the pharmaceutically acceptable salt is formulated in a pharmaceutical composition according to any one of claims 1-67.

69. The method of claim 68, wherein the anemia is anemia of chronic disease, anemia of chronic inflammation, anemia associated with cancer or fibrodysplasia ossificans progressive.

70. The method of claim 68, wherein the subject has very low, low or intermediate myelodysplastic syndrome.

71. The method of claim 68, wherein the anemia is anemia associated with MDS, transfusion dependent anemia associated with MDS, MDS with single lineage dysplasia refractory anemia, or MDS with ring sideroblasts.

72. The method of claim 68, wherein the MDS is primary MDS, secondary MDS, high-risk MDS, intermediate-risk MDS, low-risk MDS, very low, low or intermediate MD S.

73. The method of any one of claims 68-72, wherein the subject is intolerant, resistant, or refractory to luspatercept.

74. The method of claim 68, wherein the cancer is a breast cancer, ovarian cancer, prostate cancer, pancreatic cancer, or head and neck cancer.

75. The method of claim 68, wherein the cancer is a myeloproliferative disorder, a hematological cancer, or a solid tumor.

76. The method of claim 75, wherein the solid tumor is a breast tumor, ovarian tumor, prostate tumor, pancreatic tumor, or head and neck tumor.

77. The method of claim 75, wherein the solid tumor is renal cell carcinoma, or hepatocellular carcinoma.

78. The method of claim 75, wherein the myeloproliferative disorder is myelofibrosis, polycythemia vera, or essential thrombocytosis.

79. The method of claim 75, wherein the hematological cancer is lymphoma.

80. The method of claim 68, wherein the method improves one or more hematologic parameters in the subject, said improvement comprises decreasing myoblasts, increasing hemoglobin, increasing platelets, increasing neutrophils, decreasing hepcidin, reducing units of red blood cell transfused, reducing frequency of transfusion, and/or reducing transfusion dependence.

81. The method of claim 80, wherein the subject is suffering from anemia and has very low, low or intermediate myelodysplastic syndrome (MDS).

82. The method of claim 80, wherein units of red blood cells transfused is reduced by 4 or more units compared to the units of red blood cells transfused for the same period of time prior to administration of the compound of formula (I) or the pharmaceutically acceptable salt; wherein the period of time is 8 weeks or 12 weeks.

83. The method of claim 80, wherein the subject has transfusion dependent anemia associated with MDS, MDS with single lineage dysplasia refractory anemia, or MDS with ring sideroblasts and is intolerant, resistant or refractory to luspatercept.

84. The method of claim 80, wherein increasing hemoglobin is defined as increasing hemoglobin i) to 10 g/dL or more; or ii) by 1.5 g/dL or more compared to an amount measured prior to administration of the compound of formula (I) or the pharmaceutically acceptable salt;
wherein the increase in hemoglobin is maintained for 8 weeks or 12 weeks in the absence of red blood cell transfusions.

85. The method of claim 80, wherein increasing platelets is defined as increasing the platelet count i) by 30×10⁹/L or more; or ii) to 75×10⁹/L or more; wherein the increase in platelets is maintained for 8 weeks or 12 weeks in the absence of red blood cell transfusions.

86. The method of claim 80, wherein increasing neutrophils is defined as increasing the neutrophil count i) by 0.5×10⁹/L or more or ii) to 1.0×10⁹/L or more; wherein the increase in neutrophil count is maintained for 8 weeks or 12 weeks in the absence of red blood cell transfusions.

87. The method of claim 80, wherein decreasing hepcidin is defined as decreasing hepcidin by 25% or more compared to a baseline amount measured prior to administration of the compound of formula (I) or the pharmaceutically acceptable salt.

88. The method of claim 80, wherein decreasing myoblasts is defined as decreasing myoblasts i) to be 5% or fewer of bone marrow cells; or ii) by 50% or more compared to a baseline amount measured prior to administration of the compound of formula (I) or the pharmaceutically acceptable salt.

89. The method of any one of claims 68-88, wherein the compound of formula (I) or the pharmaceutically acceptable salt is administered at a daily dosage of from 10 mg to 350 mg, from 90 mg to 120 mg, 20 mg, 40 mg, 60 mg, 90 mg, 120 mg, 160 mg, 210 mg, 270 mg, or 300 mg.

90. The method of any one of claims 68-88, wherein the compound of formula (I) or the pharmaceutically acceptable salt is administered at a maintenance dosage regime, wherein the maintenance dose is the dose at which the subject achieves and maintains for a period of time a predetermined threshold level of a hemoglobin or a biomarker.

91. The method of claim 90, wherein the biomarker is selected from i) hepcidin in serum and bone marrow aspirate;
ii) iron metabolism markers in serum selected from iron, ferritin, transferrin, soluble transferrin receptor [STR], and total iron binding capacity [TIBC];
iii) cytokines in serum or plasma selected from CRP, EPO, IL-6, and TGF-beta 1; and
iv) indicators of inhibition of signal transduction pathways in bone marrow aspirates selected from phosphorylation of SMAD-1, 2, 3, 5 and 8 in PBMCs.

92. The method of any one of claims 68-91, wherein the compound of formula (I) or the pharmaceutically acceptable salt is formulated in a gelatin capsule.

93. The method of claim 92, wherein the gelatin capsule comprises about 5 mg, 25 mg, or 125 mg of the compound of formula or the pharmaceutically acceptable salt, which is based on free base weight of the compound of formula (I).

94. The method of claim 92, wherein the gelatin capsule comprises:
a) about 5.4 mg of hydrochloric acid salt of the compound of formula (I);
b) about 36.80 mg of microcrystalline cellulose;
c) about 121.00 mg of lactose;
d) about 5.10 mg of croscarmellose sodium; and
e) about 1.70 mg of magnesium stearate.

95. The method of claim 92, wherein the gelatin capsule comprises:
a) about 27.00 mg of hydrochloric acid salt of the compound of formula (I);
b) about 31.50 mg of microcrystalline cellulose;
c) about 104.70 mg of lactose;
d) about 5.10 mg of croscarmellose sodium; and
e) about 1.70 mg of magnesium stearate.

96. The method of claim 92, wherein the gelatin capsule comprises:
a) about 135.00 mg of hydrochloric acid salt of the compound of formula (I);
b) about 53.10 mg of microcrystalline cellulose;
c) about 176.70 mg of lactose;
d) about 11.40 mg of croscarmellose sodium; and
e) about 3.80 mg of magnesium stearate.

97. The method of claim 92, wherein the gelatin capsule comprises:
a) about 3.18% w/w of hydrochloric acid salt of the compound of formula (I);
b) about 21.65% w/w of microcrystalline cellulose;
c) about 71.18% w/w of lactose;
d) about 3.00% w/w of croscarmellose sodium; and
e) about 1.00% w/w of magnesium stearate.

98. The method of claim 92, wherein the gelatin capsule comprises:
a) about 15.88% w/w of hydrochloric acid salt of the compound of formula (I);
b) about 18.53% w/w of microcrystalline cellulose;
c) about 61.59% w/w of lactose;
d) about 3.00% w/w of croscarmellose sodium; and
e) about 1.00% w/w of magnesium stearate.

99. The method of claim 92, wherein the gelatin capsule comprises:
a) about 35.53% w/w of hydrochloric acid salt of the compound of formula (I);
b) about 13.97% w/w of microcrystalline cellulose;
c) about 46.50% w/w of lactose;
d) about 3.00% w/w of croscarmellose sodium; and
e) about 1.00% w/w of magnesium stearate.

100. The method of claim 92, wherein the gelatin capsule comprises:
a) about 3-3.3% w/w of hydrochloric acid salt of the compound of formula (I);
b) about 20-23% w/w of microcrystalline cellulose;
c) about 70-73% w/w of lactose;
d) about 3.00% w/w of croscarmellose sodium; and
e) about 1.00% w/w of magnesium stearate.

101. The method of claim 92, wherein the gelatin capsule comprises:
a) about 14-17% w/w of hydrochloric acid salt of the compound of formula (I);
b) about 17-20% w/w of microcrystalline cellulose;
c) about 60-64% w/w of lactose;
d) about 3.00% w/w of croscarmellose sodium; and
e) about 1.00% w/w of magnesium stearate.

102. The method of claim 92, wherein the gelatin capsule comprises:
a) about 34-37% w/w of hydrochloric acid salt of the compound of formula (I);
b) about 12-15% w/w of microcrystalline cellulose;
c) about 45-48% w/w of lactose;
d) about 3.00% w/w of croscarmellose sodium; and
e) about 1.00% w/w of magnesium stearate.

103. The method of any one of claims 68-102, wherein the pharmaceutically acceptable salt or hydrochloric acid salt is hydrochloric acid crystalline salt of the compound of formula (I).

104. The method of claim 103, wherein hydrochloric acid crystalline salt is Form A of the hydrochloric acid crystalline salt.
